(19) United States
(12) Patent Application Publication
(10) Pub. No.: US 2026/0306846 A1
(43) Pub. Date: Oct. 8, 2026

(54) Neuromodulation of Arousal Levels for the Treatment of Sleep-Wake Disorders

(71) Applicant: The Regents of the University of California, Oakland, CA (US)

(72) Inventors: Andrew Krystal, San Francisco, CA (US); Joline Marie Fan, San Francisco, CA (US)

(21) Appl. No.: 19/481,819

(22) PCT Filed: May 9, 2024
(86) PCT No.: PCT/US2024/028530

§ 371 Date: Nov. 5, 2025

Related U.S. Application Data
(60) Provisional application No. 63/465,029, filed on May 9, 2023.

(51) Int. Cl. A61N 1/36

(52) U.S. Cl. CPC A61N 1/36078; A61N 1/36139; A61N 1/36171; A61N 1/36175

(57) ABSTRACT

Methods and systems for modulating sleep-wake levels of a subject are provided. The methods include applying electrical stimulation to a location in a ventral capsule region, an orbitofrontal cortex region, or a subgenual cingulate region of the subject using an electrode in a manner effective to modulate sleep-wake levels of the subject. In addition, methods of treating sleep-wake disorders, such as hypersomnia and insomnia are provided. Both open-loop and closed-loop therapy approaches are provided for treatment of sleep-wake disorders. Closed-loop therapy is performed with a neurostimulator that records brain electrical signals from neural activity associated with wakefulness or sleepiness and delivers electrical stimulation when pre-specified patterns of neural activity associated with wakefulness or sleepiness are detected. Methods of monitoring and evaluating the effects of electrical stimulation on sleep-wake levels are also provided.

### CROSS-REFERENCE TO RELATED APPLICATION

This application claims benefit under 35 U.S.C. § 119(e) of provisional application 63/465,029, filed May 9, 2023, which application is hereby incorporated by reference in its entirety.

### INTRODUCTION

Humans routinely shift their arousal level in response to mood, stress, and reward (Saper et al. (2017) Curr. Opin. Neurobiol. 44:186, Ashbrook et al. (2020) Neuropsychopharmacology 45 (1): 45-54, Wright et al. (2012) Front. Mol. Neurosci. 5:50), complementing internal homeostatic and circadian drives. For example, stressful stimuli that give rise to aversive or rewarding outcomes may promote increased vigilance and wakefulness, whereas less motivating stimuli, such as a soporific lecture, can lead to decreased vigilance and increased sleepiness. Furthermore, in many disorders of mood, e.g. depression, anxiety, and mania, maladaptive responses to emotional factors can lead to disruptions in the control of arousal (Konjarski et al. (2018) Sleep Med. Rev. 42:47-58, Ptáček et al. (2016) Biol. Psychiatry. 80 (11): 810-811, Becker (2006) Curr. Treat Options Neurol. 2006; 8 (5): 367-375, Alvaro et al. (2013) Sleep 36 (7): 1059-1068). Nearly 90% of patients with depression are reported to have sleep-wake disturbances, including insomnia and hypersomnia. The diversity of emotional factors that modulate arousal in both adaptive and maladaptive ways suggests that the arousal circuitry may be more distributed than previously appreciated and integrated into the networks that mediate mood. Therefore, we assessed the feasibility of modulating sleep-wake levels at novel sites within classical mood-related circuitry.

### SUMMARY

Methods and systems for modulating sleep-wake levels of a subject are provided. The methods include applying electrical stimulation to a location in a ventral capsule (VC) region, an orbitofrontal cortex (OFC) region, or a subgenual cingulate (SGC) region of the subject using an electrode in a manner effective to modulate sleep-wake levels of the subject. In addition, methods of treating sleep-wake disorders, such as hypersomnia and insomnia are provided. Both open-loop and closed-loop therapy approaches may be used for treatment of sleep-wake disorders. In some embodiments, closed-loop therapy is performed with a neurostimulator that records brain electrical signals from neural activity associated with wakefulness or sleepiness and delivers electrical stimulation when pre-specified patterns of neural activity associated with wakefulness or sleepiness are detected. Methods are also disclosed for mapping subcortical and cortical stimulation responses to optimize placement of electrodes for delivery of electrical stimulation and/or to optimize placement of electrodes for detecting neural activity associated with wakefulness or sleepiness. Methods of monitoring and evaluating the effects of electrical stimulation on sleep-wake levels are also provided.

Sleep-wake disorders that can be treated by the methods disclosed herein include any disease or condition that causes problems with poor quality of sleep, timing of sleep, and/or the amount of sleep of a subject, which may result in impaired daytime functioning. Sleep-wake disorders include, but are not limited to, hypersomnia, excessive daytime sleepiness, insomnia, sleep apnea, parasomnia, narcolepsy, restless leg syndrome, non-rapid eye movement (NREM) sleep arousal disorder, and rapid and eye movement (REM) sleep behavior disorder.

In certain cases, the sleep-wake disorder is hypersomnia and applying the electrical stimulation treats hypersomnia. In certain cases, the sleep-wake disorder is insomnia and applying the electrical stimulation treats insomnia.

In certain embodiments, sleepiness or wakefulness of the subject is measured by using a visual-analog scale (VAS), a psychomotor vigilance test (PVT), a Likert scale, a Stanford Sleepiness Scale (SSS), a maintenance of wakefulness test (MWT), an Epworth sleepiness scale (ESS), a multiple sleep latency test (MSLT), or an Athens insomnia scale (AIS), wherein applying the electrical stimulation is effective in reducing sleepiness or wakefulness according to the VAS, SSS, MWT, ESS, MSLT, or AIS score.

In certain embodiments, electrical stimulation is applied unilaterally or bilaterally. In some embodiments, the electrical stimulation is applied one or more times, wherein the site to which the electrical stimulation is applied is alternated or otherwise spatially or temporally patterned.

In certain embodiments, the electrical stimulation is applied to a location in a right VC region, a left VC region, or a combination thereof.

In certain aspects, an electrical signal is measured from the brain to determine the degree of sleepiness or wakefulness of a subject by intracranial electroencephalography (iEEG) or a similar technique. In some cases, the brain electrical signal may be measured continuously, intermittently, or both. For continuous measurement, electroencephalography may be performed for an uninterrupted period of 10 sec-24 hrs or more, e.g., 30 sec-18 hrs, 1 min-12 hrs, 10 min-8 hrs, 30 sec-10 min, 1 min-10 min, 3 min-10 min, or 3 min-6 min. In some cases, a symptom of a sleep-wake disorder may be detected by the presence of increased power of gamma frequency (30 Hz to 200 Hz) or beta frequency (13 Hz to 30 Hz) electrical activity in the brain.

In certain embodiments, the electrical stimulation is applied at a frequency in a range from 70 Hz to 250 Hz, wherein wakefulness or energy of the subject is increased compared to wakefulness or energy of the subject before said applying the electrical stimulation. In some embodiments, applying electrical stimulation at a frequency in a range from 70 Hz to 250 Hz decreases reaction time of the subject (as can be assessed by a psychomotor vigilance test) compared to the reaction time of the subject before said applying the electrical stimulation.

In other embodiments, the electrical stimulation is applied at a frequency in a range of 0.5 Hz to 10 Hz, wherein sleepiness of the subject is increased compared to sleepiness of the subject before said applying the electrical stimulation. In some embodiments, applying electrical stimulation at a frequency in a range from 0.5 Hz to 10 Hz increases reaction time of the subject (as can be assessed by a psychomotor vigilance test) compared to the reaction time of the subject before said applying the electrical stimulation.

The stimulation electrode may be a brain-penetrating electrode or a non-brain penetrating electrode. The detection electrode may be a brain-penetrating electrode or a non-brain penetrating electrode. Electrical stimulation may be applied as described herein.

In certain embodiments, the electrical stimulation is applied for 5 seconds to 120 seconds. In some embodiments, the electrical stimulation is applied intermittently with a rest period between stimulations. In some embodiments, the rest period ranges from 10 seconds to 10 minutes. For example, the electrical stimulation may be applied intermittently with a stimulation period of 6 seconds and a rest period of 10 seconds. In another example, the electrical stimulation may be applied intermittently with a stimulation period of 30 seconds and a rest period of 2 minutes.

In certain embodiments, the method further comprises mapping the VC region, the OFC region, or the SGC region of the subject to identify an optimal location for positioning the electrode for delivery of the electrical stimulation to modulate sleep-wake levels of the subject.

In certain embodiments, the electrode is placed on a surface of the VC region, the OFC region, or the SGC region. In certain embodiments, the electrode is placed within the VC region, the OFC region, or the SGC region.

In certain embodiments, the method further comprises monitoring wakefulness or sleepiness of the subject by recording a brain electrical signal associated with wakefulness or sleepiness of the subject, wherein the brain electrical signal is from a ventral capsule (VC) region, a subgenual cingulate (SGC) region, an amygdala region, an orbitofrontal cortex (OFC) region, or a striato-limbic region of the subject. In some embodiments, the brain electrical signal associated with wakefulness or sleepiness comprises alpha frequency, beta frequency, gamma frequency, delta frequency, or theta frequency neural oscillations. In some embodiments, the gamma frequency neural oscillations are low gamma frequency neural oscillations in a range from 31 Hz to 81 Hz or high gamma frequency neural oscillations in a range from 81 Hz to 150 Hz. In some embodiments, the method further comprises measuring a relative spectral power level for the gamma frequency neural oscillations, wherein higher relative power of the gamma frequency neural oscillations is associated with lower sleepiness and higher wakefulness compared to lower relative power of the gamma frequency neural oscillations. In some embodiments, the method further comprises measuring a relative spectral power level for the beta frequency neural oscillations, wherein higher relative power of the beta frequency neural oscillations is associated with lower sleepiness and higher wakefulness compared to lower relative power of the beta frequency neural oscillations.

In certain embodiments, electrical stimulation is applied when a recorded brain electrical signal associated with wakefulness or sleepiness of the subject exceeds a threshold level. In some embodiments, the subject is monitored continuously or intermittently.

In certain embodiments, applying electrical stimulation comprises applying non-invasive brain stimulation to the VC region, the OFC region, or the SGC region. In some embodiments, non-invasive brain stimulation is applied to a right VC region, a left VC region, or a combination thereof.

In another aspect, a closed-loop method for treating hypersomnia in a subject is provided, the method comprising: recording a brain electrical signal associated with sleepiness of the subject using a first electrode, wherein the brain electrical signal is from a ventral capsule (VC) region, a subgenual cingulate (SGC) region, an amygdala region, an orbitofrontal cortex (OFC) region, or a striato-limbic region of the subject; and applying electrical stimulation to a location in the VC region, the OFC region, or the SGC region of the subject using a second electrode in a manner effective to treat the hypersomnia in the subject when the electrical signal that is detected using the first electrode exceeds a threshold level. In certain embodiments, the electrical stimulation is applied to a location in a right VC region, a left VC region, or a combination thereof. In some embodiments, the electrical stimulation is applied at a frequency in a range from 70 Hz to 250 Hz such that wakefulness or energy of the subject is increased compared to wakefulness or energy of the subject before said applying the electrical stimulation. Applying the electrical stimulation at a frequency in a range from 70 Hz to 250 Hz may also decrease reaction time of the subject compared to the reaction time of the subject before applying the electrical stimulation, as can be shown in a psychomotor vigilance test. In some embodiments, the electrical stimulation is applied for 5 seconds to 120 seconds.

In certain embodiments, the method further comprises mapping the ventral capsule (VC) region, a subgenual cingulate (SGC) region, an amygdala region, an orbitofrontal cortex (OFC) region, or a striato-limbic region of the subject to identify an optimal location for positioning the first electrode for recording the brain electrical signal associated with sleepiness of the subject.

In certain embodiments, the method further comprises mapping the VC region, the OFC region, or the SGC region of the subject to identify an optimal location for positioning the second electrode for delivery of the electrical stimulation to treat the hypersomnia of the subject.

In certain embodiments, the method further comprises using a control algorithm to automate said applying electrical stimulation when the brain electrical signal associated with sleepiness exceeds the threshold level. In some embodiments, the control algorithm comprises a machine learning algorithm for classification of degree of sleepiness based on the brain electrical signal. In some embodiments, the machine learning algorithm is a supervised machine learning algorithm. In some embodiments, the control algorithm further modulates one or more programmed stimulation parameters based on a level of power of the brain electrical signal.

In certain embodiments, the brain electrical signal associated with sleepiness comprises alpha frequency, beta frequency, gamma frequency, delta frequency, or theta frequency neural oscillations. In some embodiments, the brain electrical signal associated with sleepiness comprises low gamma frequency neural oscillations in a range from 31 Hz to 81 Hz or high gamma frequency neural oscillations in a range from 81 Hz to 150 Hz, or a combination thereof. In some embodiments, the method further comprises measuring a relative spectral power level for the gamma frequency neural oscillations, wherein higher relative power of the gamma frequency neural oscillations is associated with lower sleepiness and higher wakefulness compared to lower relative power of the gamma frequency neural oscillations.

In certain embodiments, the method further comprises assessing effectiveness of the electrical stimulation in treating the hypersomnia in the subject.

In another aspect, a closed-loop method for treating insomnia in a subject is provided, the method comprising: recording a brain electrical signal associated with wakefulness of the subject using a first electrode, wherein the brain electrical signal is from a ventral capsule (VC) region, a subgenual cingulate (SGC) region, an amygdala region, an orbitofrontal cortex (OFC) region, or a striato-limbic region of the subject; and applying electrical stimulation to a location in the VC region, the OFC region, or the SGC region of the subject using a second electrode in a manner effective to treat the insomnia in the subject when the electrical signal that is detected using the first electrode exceeds a threshold level. In certain embodiments, the electrical stimulation is applied to a location in a right VC region, a left VC region, or a combination thereof. In some embodiments, the electrical stimulation is applied at a frequency in a range of 0.5 Hz to 10 Hz such that sleepiness of the subject is increased compared to sleepiness of the subject before said applying the electrical stimulation. In some embodiments, the electrical stimulation is applied for 5 seconds to 120 seconds.

In certain embodiments, the method further comprises mapping the ventral capsule (VC) region, a subgenual cingulate (SGC) region, an amygdala region, or an orbitofrontal cortex (OFC) region of the subject to identify an optimal location for positioning the first electrode for recording the brain electrical signal associated with wakefulness of the subject.

In certain embodiments, the method further comprises mapping the VC region, the OFC region, or the SGC region of the subject to identify an optimal location for positioning the second electrode for delivery of the electrical stimulation to treat the insomnia in the subject.

In certain embodiments, the method further comprises using a control algorithm to automate said applying electrical stimulation when the brain electrical signal associated with wakefulness exceeds the threshold level. In some embodiments, the control algorithm comprises a machine learning algorithm for classification of degree of wakefulness based on the brain electrical signal. In some embodiments, the machine learning algorithm is a supervised machine learning algorithm. In some embodiments, the control algorithm further modulates one or more programmed stimulation parameters based on a level of power of the brain electrical signal.

In certain embodiments, the brain electrical signal associated with wakefulness comprises alpha frequency, beta frequency, gamma frequency, delta frequency, or theta frequency neural oscillations. In some embodiments, the brain electrical signal associated with wakefulness comprises low gamma frequency neural oscillations in a range from 31 Hz to 81 Hz or high gamma frequency neural oscillations in a range from 81 Hz to 150 Hz. In some embodiments, the method further comprises measuring a relative spectral power level for the gamma frequency neural oscillations, wherein higher relative power of the gamma frequency neural oscillations is associated with lower sleepiness and higher wakefulness compared to lower relative power of the gamma frequency neural oscillations. In some embodiments, the subject is monitored continuously or intermittently.

In some cases, the insomnia is caused by deep brain stimulation therapy to an anterior limb of the internal capsule (ALIC) region (e.g., insomnia caused by DBS treatment of obsessive-compulsive disorder or major depressive disorder).

In certain embodiments, the method further comprises assessing effectiveness of the electrical stimulation in treating the insomnia in the subject.

In another aspect, a system for modulating sleep-wake levels in a subject is provided, the system comprising: a first electrode adapted for recording a brain electrical signal associated with wakefulness or sleepiness of the subject, wherein the brain electrical signal is from a ventral capsule (VC) region, a subgenual cingulate (SGC) region, an amygdala region, an orbitofrontal cortex (OFC) region, or a striato-limbic region of the subject; a second electrode adapted for applying electrical stimulation to a location in the VC region, the OFC region, or the SGC region of the subject; and a processor programmed to instruct the second electrode to apply the electrical stimulation to the VC region, the OFC region, or the SGC region of the subject in a manner effective to modulate sleep-wake levels in the subject when the brain electrical signal associated with wakefulness or sleepiness of the subject that is detected using the first electrode exceeds a threshold level.

In certain embodiments, the system further comprises a user interface comprising an input electronically coupled to the processor for instructing the second electrode to apply the electrical stimulation to the location in the VC region, the OFC region, or the SGC region of the subject. In some embodiments, the user interface is password protected and is operable by a health care practitioner.

In certain embodiments, the processor is further programmed to modulate one or more programmed stimulation parameters according to the algorithm's control law; and apply the modulated electrical stimulation to the VC region, the OFC region, or the SGC region of the subject using the second electrode in a manner effective to treat a sleep-wake disorder.

In certain embodiments, the processor is further programmed to apply the electrical stimulation at a frequency in a range from 70 Hz to 250 Hz such that wakefulness or energy of the subject is increased and/or reaction time of the subject is decreased compared to wakefulness, energy, or reaction time of the subject before said applying the electrical stimulation.

In certain embodiments, the processor is further programmed to apply the electrical stimulation at a frequency in a range of 0.5 Hz to 10 Hz such that sleepiness and/or reaction time of the subject is increased compared to sleepiness and/or reaction time of the subject before said applying the electrical stimulation.

In certain embodiments, the processor is further programmed to apply the electrical stimulation for 5 seconds to 120 seconds.

In certain embodiments, the processor is further programmed to apply the electrical stimulation intermittently with a rest period between stimulations. In some embodiments, the rest period ranges from 10 seconds to 10 minutes. For example, the processor may be programmed to apply the electrical stimulation intermittently with a stimulation period of 6 seconds and a rest period of 10 seconds. In another example, the processor may be programmed to apply the electrical stimulation intermittently with a stimulation period of 30 seconds and a rest period of 2 minutes.

In certain embodiments, the processor is further programmed to set a maximum number of electrical stimulations per day.

In certain embodiments, the processor is further programmed to set a total amount of time of electrical stimulation per day.

In certain embodiments, the first electrode is adapted for positioning on a surface of the VC region, the SGC region, the amygdala region, or the OFC region of the subject or within the VC region, the SGC region, the amygdala region, or the OFC region of the subject.

In certain embodiments, the second electrode is adapted for positioning on a surface of the VC region, the OFC region, or the SGC region or within the VC region, the OFC region, or the SGC region.

In certain embodiments, the first electrode or the second electrode is a non-brain penetrating surface electrode array or a brain-penetrating electrode array.

In certain embodiments, the first electrode or the second electrode is a non-brain penetrating electrode.

In certain embodiments, the first electrode or the second electrode is a brain penetrating electrode.

In certain embodiments, the second electrode applies non-invasive brain stimulation (NIBS) to the VC region, the OFC region, or the SGC region. In some embodiments, the second electrode applies non-invasive brain stimulation (NIBS) to the right VC region, the left VC region, or a combination thereof.

In certain embodiments, a time period for electrical stimulation and a rest period free of electrical stimulation may be set for the subject. For example, for a subject who has hypersomnia, a nighttime rest period without electrical stimulation may be employed to avoid interfering with sleep when the subject wishes to sleep at night, and a daytime stimulation period may be employed, wherein electrical stimulation is used to improve wakefulness and energy durng the day. Conversely, for a subject who has insomnia, a nighttime stimulation period may be employed, wherein electrical stimulation is used to promote sleepiness to allow a subject to sleep when the subject wishes to sleep at night, and a daytime rest period may be employed when such stimulation is unnecessary. In some cases, the frequency of stimulation may be adjusted depending on the sleep-wake levels of the subject, wherein different frequencies of stimulation may be used during different time periods as needed. For example, electrical stimulation may be applied at a frequency in a range from 70 Hz to 250 Hz to increase daytime wakefulness or energy of the subject when needed, and electrical stimulation may be applied at a frequency in a range of 0.5 Hz to 10 Hz to increase nighttime sleepiness of the subject when needed.

In some embodiments, electrical activity in the beta frequency range (such as 13 Hz to 30 Hz) may be measured, wherein increases in spectral power of the beta frequency electrical activity is correlated with wakefulness or insomnia, and electrical stimulation may be applied at a frequency in a range of 0.5 Hz to 10 Hz to increase nighttime sleepiness of the subject when needed. In some embodiments, electrical stimulation in a range of 0.5 Hz to 10 Hz is applied during nighttime hours when spectral power of the beta frequency brain electrical signal exceeds a threshold level.

In another aspect, a closed-loop method for treating obsessive-compulsive disorder or major depressive disorder in a subject, the method comprising: applying electrical stimulation to an anterior limb of internal capsule (ALIC) region of the subject using a first electrode in a manner effective to treat the obsessive-compulsive disorder or major depressive disorder in the subject; recording a beta frequency brain electrical signal associated with wakefulness of the subject using a second electrode, wherein the beta frequency brain electrical signal is from a striato-limbic region of the subject; and decreasing amplitude of electrical stimulation during nighttime hours when spectral power of the beta frequency brain electrical signal exceeds a threshold level.

In certain embodiments, the beta frequency brain electrical signal is in a range from 25 Hz to 30 Hz.

In certain embodiments, electrical stimulation in a range of 0.5 Hz to 10 Hz is applied during nighttime hours when spectral power of the beta frequency brain electrical signal exceeds the threshold level.

### BRIEF DESCRIPTION OF THE DRAWINGS

FIGS. 1A-1B. Overview of experimental methodology and imaging of implantation sites. FIG. 1A) Following electrode implantation, each patient underwent an intensive 10 d inpatient monitoring period, in which biomarker recordings and stimulation testing were performed. Biomarker recordings comprised of 10 minutes of resting state activity, followed by survey completion. During stimulation testing, patients provided spontaneous verbal reports of any sensations and survey responses were completed at the end of the stimulation period. Survey assessments included the SSS and VAS-Energy scores. In addition, bedside clinical arousal scores were recorded in response to stimulation. FIG. 1B) Structural connectivity analysis for both patients based on seeding the normative connectome with the volume of tissue predicted to be activated by stimulation at representative stimulation sites within the VC (top), SGC (middle), and OFC (bottom). Stimulation at VC contacts is associated with fibers corresponding to the medial forebrain bundle (MFB), anterior thalamic radiations (ATR), and ansa peduncularis (AP). Stimulation at SGC contacts is associated with fibers corresponding to the forceps minor (FM), cingulum bundle (CB), and uncinate fasciculus (UF). Stimulation at OFC contacts is associated with fibers corresponding to the ventral component of the ATR, UF, and frontal asalant (FA) tracts. Red spheres represent the location and volume of tissue activated used for fiber tracking in the normative connectome. Coloration of tracts follows conventional coding, i.e., right-left (red), anterior-posterior (green), and superior-inferior (blue) fiber directions.

FIGS. 2A-2D. Example behavioral reports and survey responses from an individual patient. FIG. 2A) Example spontaneous verbal reports associated with stimulation at the specified regions for Patient 1. FIGS. 2B-2D) Survey responses from Patient 2 for arousal, energy, and SSS, respectively, illustrate a statistically significant increase in energy and reduction in sleepiness in response to right VC 100 Hz stimulation, and a reduction in sleepiness in response to left OFC 100 Hz stimulation. Different electrode bipolar pairs within a region of stimulation are uniquely color-coded within each violin plot. Abbreviations: R=right; L=left; OFC=orbital frontal cortex; SGC=subgenual cingulate, NAC=nucleus accumbens, VC=ventral capsule; Amy=amygdala.

FIG. 3. Behavioral effects of arousal to regional stimulation across in both patients. Stimulation response quantified by the summary metric (i.e. first dimension of PCA across the three behavioral assessments) in Patients 1 (left) and 2 (right), respectively. Different electrode bipolar pairs within a region of stimulation are uniquely color-coded within each violin plot.

FIGS. 4A-4H. Electrophysiologic biomarkers of sleepiness across both patients. FIG. 4A) Electrophysiology for biomarker analysis was obtained from the resting-state periods preceding survey completion. FIG. 4B) Top 10 features with highest positive and negative correlations with SSS for Patient 1. Correlations that were statistically significant following multiple comparison testing across regions are indicated by the line and asterisks (right). FIG. 4C) Same as FIG. 4B, but for Patient 2. FIG. 4D) Top 20 features by magnitudes, averaged across both patients. Statistically significant correlations that are common across the two patients are denoted by the bar and asterisks (right). FIG. 4E) Example of highest positive and negative correlations between SSS and candidate electrophysiologic features, e.g., relative power in the alpha band of the right hippocampus (top) and low gamma band of the left ventral capsule (bottom). Purple and orange background shading represent the high and low sleepiness state cut-offs used for logistic regression classification. FIG. 4F) Same as FIG. 4E, but for Patient 2, illustrating the relationship between SSS and relative power in the delta band of the right OFC (top) and high gamma band of the left hippocampus (bottom). FIG. 4G) ROC curves representing the best average model from cross-validated logistic regressions using six features (top) with AUC 0.78. Demonstration of top model features used in multivariate logistic regression (bottom), quantified by percent of models in which features were selected from cross-validated 1000 iterations. FIG. 4H) Same as FIG. 4G, but for Patient 2 for whom the best logistic regression model achieved an AUC of 0.92.

FIGS. 5A-5D. Effects of regional stimulation on shared electrophysiologic biomarkers of sleepiness. FIG. 5A) Example local spectrograms before, during, and after stimulation between the left VC 2 and 3 channels at 100 Hz, 2 mA. Green and red lines denote onset and offset of stimulation, respectively. Visualized spectrograms represent the electrophysiology observed from the left VC (top left), left Amy (top right), left SGC (bottom left) and right OFC (bottom right) contacts. FIG. 5B) Changes from pre-stim to post-stim z-scored spectral power for identified biomarkers of sleepiness aggregated across both patients, in response to VC stimulation (cyan dots, patient 1; blue dots, patient 2; red dots, group mean). A subset of the group biomarker responses to stimulation are presented, including high gamma power within the left SGC, left VC, left Amy and right OFC. FIG. 5C)) Example local spectrograms during stimulation between the right OFC 2 and 9 channels at 1 Hz, 3 mA. Visualized spectrograms represent the electrophysiology from the same contacts as in FIG. 5A. FIG. 5D) As in FIG. 5B, change from pre-stim to post-stim high gamma power within the left SGC, left VC, left Amy, and right OFC, in response to 1 Hz OFC stimulation. No statistically significant responses were observed in the low or high gamma power with respect to 1 Hz OFC stimulation.

FIG. 6. Correlation across behavioral outcome measures (VAS-Energy, SSS, and arousal) for an individual patient (Pearson's correlation between energy and arousal is 0.69, p<0.001; Patient 2). Pearson's correlation coefficients for Patient 1 (right, top) and Patient 2 (right, bottom) are denoted.

FIG. 7. Regional stimulation responses of PCA-1 in the initial patient. Different electrode bipolar pairs within a region of stimulation are uniquely color-coded within each violin plot. Abbreviations: R=right; L=left; OFC=orbital frontal cortex; SGC=subgenual cingulate, VC=ventral capsule; Amy=amygdala.

FIG. 8. Dose responses of arousal scores across different amplitudes of stimulation for the right OFC 1 Hz, right OFC 100 Hz, right SGC 100 Hz, and right VC 100 Hz. Arousal responses represent the least-square mean and 95% confidence limits from a linear mixed model of arousal measures across all three patients.

FIGS. 9A-9K. Striato-limbic beta power shows consistent circadian fluctuation and is enhanced by ALIC DBS. FIG. 9A) Bilateral electrode placement for all four subjects in MNI space. Recording contacts are highlighted in red. FIG. 9B) 7-week continuous recording of band-limited power within a 25.39±2.5 Hz range averaged in 10-min intervals within the striato-limbic from an example subject (lead location marked by X in FIG. 9A). FIG. 9C) Daily detrended striato-limbic beta power vs clock time across recording from the example subject. FIG. 9D) Periodogram (normalized) of the striato-limbic beta power recordings. Thin gray lines show individual power spectra (n=7) and black line shows the mean across signals. FIG. 9E) Average normalized and detrended striato-limbic beta power vs. clock time for each recording electrode (thin gray lines, n=7). Average power across recordings from all subjects represented by thick black line. FIG. 9F) Average (solid line) and individual (thin gray lines, n=7) detrended striato-limbic beta power recordings around the circadian cycle. FIG. 9G) Sinusoidal regression coefficients for mean striato-limbic beta power vs. clock time. FIG. 9H) Spectrogram of the left ALIC recording, which represents the active stimulation lead. Spectral power is represented by the z-score to the initial baseline. FIG. 9I) Violin plots representing VAS-energy to high and low stimulation amplitudes, across OCD1 (black dots) and OCD4 (blue dots). Group mean is denoted by black line; group median is denoted by red line. Significance determined by Wilcoxon rank sum test. FIG. 9J) Scatter plots demonstrating the relationship between z-scored striato-limbic beta power of the active stimulation lead (left ALIC) and VAS-energy across patients. Linear regression is performed across patients with Pearson's correlation denoted. Red lines denote linear regression model with 95% confidence intervals represented. FIG. 9K) Relationship between z-scored striato-limbic beta power of the contralateral sensing lead (right ALIC) and VAS-energy across patients. (For interpretation of the references to colour in this figure legend, the reader is referred to the Web version of this article.)

FIGS. 10A-10C. Sleepiness survey measures and striato-limbic beta power changes with high and low stimulation paradigms across two patients. FIG. 10A) SSS scores between low and high L ALIC DBS across OCD1 (black dots) and OCD4 (blue dots). Non-significance assessed by Wilcoxon rank sum test. FIG. 10B) Stimulation-induced changes in z-scored beta power in the left, active stimulation lead (left panel) and the right, sensing lead (right panel) with low and high stimulation amplitudes in the ventral ALIC. Significance determined by Wilcoxon rank sum test. FIG. 10C) Scatter plots demonstrating the relationship between z-scored beta power and SSS across patients. Correlations are assessed using Pearson's correlation.

FIGS. 11A-11C. Stimulation-induced behavioral changes and relationship between behavior and striato-limbic beta power across two subjects. FIG. 11A) Violin plots representing PCA1 of SSS/VAS-E scores of high and low stimulation amplitudes, across OCD1 (black dots) and OCD4 (blue dots). Group mean is denoted by black line; group median is denoted by red line. Significance determined by Wilcoxon rank sum test. FIG. 11B) Scatter plots demonstrating the relationship between z-scored striato-limbic beta power of the active stimulation lead (left ALIC) and PCA1 of SSS/VAS-E across patients. Linear regression is performed across patients with Pearson's correlation denoted. Red lines denote linear regression model with 95% confidence intervals represented. FIG. 11C) Relationship between z-scored striato-limbic beta power of the contralateral sensing lead (right ALIC) and PCA1 of SSS/VAS-E across patients.

FIG. 12. Reaction times vs. SSS scores. We assessed whether increased energy/wakefulness translated into improved reaction times in a standardized psychomotor vigilance test. This is a well-known assessment that captures the reaction time as a surrogate of vigilance. First, we demonstrated that across all baseline PVTs vs. closest SSS survey, there is a positive, statistically significant relationship between reaction time (RT) and Stanford sleepiness score (SSS), indicating that that RT here reflects the degree of sleepiness.

FIG. 13. Effect of stimulation on reaction times in a patient. We performed sequential stimulation and PVT testing in serial assessments, e.g., Block A-C/D, altering stimulation conditions to assess stimulation effects.

FIG. 14. Effects of stimulation on behavioral score changes and PVT results. OFC stimulation at 1 Hz increased the RT based on the PVT results (i.e., slows reaction time), while specific conditions of VC stimulation and OFC stimulation at 100 Hz decreased the RT (i.e. quickens the reaction time). In contrast, sham slows reaction time, which is not surprising given that persistent testing of the PVT is likely to lead decreased vigilance over time.

FIG. 15. SSS, VAS-Depression, and VAS-anxiety scores before and after stimulation. The subject demonstrated that reduction of sleepiness (enhancement of wakefulness) is attainable in absence of altering other mood measures, such as depression and anxiety. This was manifested where we pulled together pre and post stimulation responses from an initial stimulation to VC stimulation. In this context, the patient reports comments such as “I think I feel the energy” or is witnessed to go from the recumbent position with eyes closed to then getting up and opening her laptop.

### DETAILED DESCRIPTION

Methods and systems for modulating sleep-wake levels of a subject are provided. The methods include applying electrical stimulation to a location in a ventral capsule (VC) region, an orbitofrontal cortex (OFC) region, or a subgenual cingulate (SGC) region of the subject using an electrode in a manner effective to modulate sleep-wake levels of the subject. In addition, methods of treating sleep-wake disorders, such as hypersomnia and insomnia are provided. Both open-loop and closed-loop therapy approaches may be used for treatment of sleep-wake disorders. In some embodiments, closed-loop therapy is performed with a neurostimulator that records brain electrical signals from neural activity associated with wakefulness or sleepiness and delivers electrical stimulation when pre-specified patterns of neural activity associated with wakefulness or sleepiness are detected. Methods are also disclosed for mapping subcortical and cortical stimulation responses to optimize placement to optimize placement of electrodes for delivery of electrical stimulation and/or to optimize placement of electrodes for detecting neural activity associated with wakefulness or sleepiness. Methods of monitoring and evaluating the effects of electrical stimulation on sleep-wake levels are also provided.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an electrode” or “the electrode” includes a plurality of such electrodes and reference to “a symptom” or “the symptom” includes reference to one or more symptoms, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The term “sleep-wake disorder” is used herein to refer to any disease or condition that causes problems with poor quality of sleep, timing of sleep, and/or the amount of sleep of a subject, which may result in impaired daytime functioning. Sleep-wake disorders include, but are not limited to, hypersomnia, excessive daytime sleepiness, insomnia, sleep apnea, parasomnia, narcolepsy, restless leg syndrome, non-rapid eye movement (NREM) sleep arousal disorder, and rapid and eye movement (REM) sleep behavior disorder.

The term “hypersomnia” is used to refer herein to a sleep-wake disorder that causes excessive time spent sleeping or excessive sleepiness. Hypersomnia may be caused by idiopathic hypersomnia, narcolepsy, Kleine-Levin syndrome, Prader-Willi syndrome, Norrie disease, Niemann-Pick disease type C, myotonic dystrophy, fibromyalgia, chronic fatigue syndrome, lupus, rheumatoid arthritis, Morvan's syndrome, hypothyroidism, multiple sclerosis, encephalitis, Alzheimer's disease, Parkinson's disease, hydrocephalus, anemia, encephalitis lethargica, paraneoplastic syndrome, head trauma, or depression.

The term “insomnia” is used to refer herein to a sleep-wake disorder that causes sleeplessness, difficulty falling asleep, or difficulty staying asleep for a sufficient period of time. Insomnia may include, but is not limited to, sleep onset insomnia, middle-of-the-night awakening, early morning awakening, and/or poor sleep quality (e.g., not reaching stage 3 or delta sleep). Insomnia may be caused by psychological stress, chronic pain, heart failure, hyperthyroidism, sleep apnea, heartburn, restless leg syndrome, menopause, or amphetamines or other stimulants. In some cases, insomnia is caused by deep brain stimulation therapy (e.g., insomnia caused by DBS to an anterior limb of the internal capsule (ALIC) region such as used for treatment of obsessive-compulsive disorder or major depressive disorder).

By “treatment” or “treating” is meant that at least an amelioration of one or more symptoms associated with the condition afflicting the subject is achieved such that the patient has a desired or beneficial clinical result, where amelioration refers to at least a reduction in the magnitude of a parameter, e.g., a symptom, associated with the condition being treated. As such, treatment includes a broad spectrum of situations ranging from lessening intensity, duration or extent of impairment caused by a condition and/or correlated with a condition, up to and including completely eliminating the condition, along with any associated symptoms. Treatment therefore includes situations where the condition, or at least a symptom associated therewith, is completely inhibited, e.g., prevented from happening, or stopped, e.g., terminated, such that the subject no longer suffers from the condition, or at least the symptoms that characterize the condition. Treatment also includes situations where the progression of the condition, or at least the progression of a symptom associated therewith, is slowed, delayed, or halted. In such cases, a subject might still have residual symptoms associated with a sleep-wake disorder, but any increase in the severity or magnitude of the symptoms is slowed, delayed, or prevented.

The term “subject” as used herein refers to a patient in need of the treatments disclosed herein. The patient may be a mammal, such as, a rodent, a feline, a canine, a primate, or a human, e.g., a child, an adolescent, an adult, such as a young, middle-aged, or elderly human. The patient may have been diagnosed as having a sleep-wake disorder, may be suspected of suffering from a sleep-wake disorder, or may be at risk of developing a sleep-wake disorder.

The term “user” as used herein refers to a person that interacts with a device and/system disclosed herein for performing one or more steps of the presently disclosed methods. The user may be the patient receiving treatment. The user may be a health care practitioner, such as, the patient's physician.

The term “symptom” as used in the context of a sleep-wake disorder, may include, without limitation, problems with poor quality of sleep, timing of sleep, and/or the amount of sleep of a subject, which may result in impaired daytime functioning. For example, a symptom of hypersomnia may include excessive time spent sleeping or excessive sleepiness. A symptom of insomnia may include sleeplessness, difficulty falling asleep, difficulty staying asleep, or poor sleep quality (e.g., not reaching stage 3 or delta sleep).

### Methods

The present disclosure provides methods for modulating sleep-wake levels of a subject. Also provided herein are methods for treating a sleep-wake disorder in a subject and methods for ameliorating a symptom of a sleep-wake disorder in a subject. Various steps and aspects of the methods will now be described in greater detail below.

Methods of the present disclosure include positioning an electrode to deliver electrical stimulation (i.e., a stimulation electrode) to a ventral capsule (VC) region, an orbitofrontal cortex (OFC) region, a subgenual cingulate (SGC) region, or other region of the brain of a subject to modify sleep-wake levels of a subject. Closed-loop therapy methods may further include positioning a second electrode to detect a brain electrical signal associated with wakefulness or sleepiness of the subject (i.e., detection electrode), wherein the electrical signal is from a VC region, SGC region, amygdala region, OFC region, striato-limbic region, or other region of the brain of the subject. One or more stimulation or detection electrodes may be used. The stimulation and detection electrodes may be positioned in the same brain region or in different brain regions. The stimulation and detection electrodes may be non-brain penetrating surface electrodes or brain-penetrating depth electrodes.

The electrical stimulation may be applied to the VC region, OFC region, SGC region, or other region of the brain of a subject using the stimulation electrode in a manner effective for modulating the sleep-wake levels of a subject and/or treating a sleep-wake disorder, which may include any disease or condition that causes problems with poor quality of sleep, timing of sleep, and/or the amount of sleep of a subject resulting in impaired daytime functioning. Sleep-wake disorders include, but are not limited to, hypersomnia, excessive daytime sleepiness, insomnia, sleep apnea, parasomnia, narcolepsy, restless leg syndrome, non-rapid eye movement (NREM) sleep arousal disorder, and rapid and eye movement (REM) sleep behavior disorder.

The electrical stimulation may ameliorate a symptom of a sleep-wake disorder. For example, the electrical stimulation may ameliorate a symptom of hypersomnia, which may include excessive time spent sleeping and/or excessive daytime sleepiness. Alternatively, the electrical stimulation may ameliorate a symptom of insomnia, which may include, difficulty falling asleep, middle-of-the-night awakening, early morning awakening, poor sleep quality (e.g., not reaching stage 3 or delta sleep), and/or excessive nighttime wakefulness.

The electrical stimulation may be applied using a single electrode, electrode pairs, or an electrode array. Electrical stimulation may be applied unilaterally or bilaterally. Bilateral electrical stimulation may be simultaneous or sequential. In some embodiments, the electrical stimulation is applied to more than one site. The site to which the electrical stimulation is applied may be alternated or otherwise spatially or temporally patterned.

Positioning an electrode for applying electrical stimulation at specified region(s) of the brain may be carried out using standard surgical procedures for placement of intra-cranial electrodes. As used herein, the phrases “an electrode” or “the electrode” refer to a single electrode or multiple electrodes such as an electrode array. As used herein, the term “contact” as used in the context of an electrode in contact with a region of the brain refers to a physical association between the electrode and the region. In other words, an electrode that is in contact with a region of the brain is physically touching the region of the brain. An electrode in contact with a region of the brain can conduct electricity into the brain. Electrodes used in the methods disclosed herein may be monopolar (cathode or anode) or bipolar (e.g., having an anode and a cathode). The electrode(s) used for applying electric stimulation to the specified region(s) of the brain are also referred to as stimulation electrode(s).

In certain embodiments, the brain region to which electrical stimulation is applied comprises the VC region, OFC region, or SGC region, or other regions of the brain suitable for stimulation. In some embodiments, the electrical stimulation is applied to a location in a right VC region, a left VC region, or a combination thereof. The site chosen for stimulation may differ for different subjects and will depend on mapping subcortical and cortical stimulation responses of an individual subject to identify the optimal location for positioning an electrode for delivery of electrical stimulation to modulate sleep-wake levels and/or treat a sleep-wake disorder, as discussed further below.

In certain cases, placing the electrode at the VC region, OFC region, or SGC, or other region may involve positioning the electrode on the surface of the specified region(s) of the brain. Electrodes may be placed on the surface of the brain at the VC, for example, in a right VC region, a left VC region, or both. The electrode may contact at least a portion of the surface of the brain at the VC, OFC, or SGC, or other region. In some embodiments, the electrode may contact substantially the entire surface area at the VC, OFC, or SGC, or other region. In some embodiments, the electrode may additionally contact area(s) adjacent to the VC, OFC, or SGC. In some embodiments, an electrode array, arranged on a planar support substrate, may be used for electrically stimulating the VC, OFC, or SGC region, or other region of the brain as specified herein. The surface area of the electrode array may be determined by the desired area of contact between the electrode array and the brain. An electrode for implanting on a brain surface, such as, a surface electrode or a surface electrode array may be obtained from a commercial supplier. A commercially obtained electrode/electrode array may be modified to achieve a desired contact area. In some cases, the non-brain penetrating electrode (also referred to as surface electrode) that may be used in the methods disclosed herein may be an electrocorticography (ECoG) electrode or an electroencephalography (EEG) electrode.

In certain cases, placing the electrode at a target area or site (e.g., a stimulation electrode) may involve positioning a brain penetrating electrode (also referred to as depth electrode) in the specified region(s) of the brain. For example, a stimulation electrode may be placed in the VC, OFC, or SGC region, or another region. In some embodiments, the stimulation electrode may additionally contact area(s) adjacent to the VC, OFC, or SGC. In some embodiments, an electrode array may be used for electrically stimulating the VC, OFC, or SGC region, or another region of the brain as specified herein.

The depth to which a stimulation electrode is inserted into the brain may be determined by the desired level of contact between the electrode array and the brain. A brain-penetrating electrode array may be obtained from a commercial supplier. A commercially obtained electrode array may be modified to achieve a desired depth of insertion into the brain tissue.

In certain embodiments of the present methods, electrical stimulation is applied only to the VC region in the brain. In some embodiments, electrical stimulation is applied only to the right VC region, only to the left VC region, or to both the right and left VC regions. In other embodiments, electrical stimulation is applied at least to the VC region in the brain and one or more additional regions in the brain. In certain embodiments, one or more detection electrodes are used to record brain electrical signals for neural activity associated with a symptom of a sleep-wake disorder in one or more brain regions. A detection electrode may be placed, for example, in the VC, SGC, amygdala, OFC, or striato-limbic region regions of the brain, or in other regions of the brain suitable for detection. The site chosen for detection may differ for different subjects and will depend on mapping subcortical and cortical stimulation responses of an individual subject to identify the optimal location for positioning an electrode for detecting brain electrical signals from neural activity associated with a symptom of a sleep-wake disorder (e.g., excessive daytime sleepiness or lack of energy, excessive nighttime wakefulness, inability to fall asleep or stay asleep long enough), as discussed further below.

In certain cases, placing the detection electrode may involve positioning the electrode on the surface of the specified region(s) of the brain. For example, electrodes may be placed on the surface of the brain at the VC, SGC, amygdala, OFC, or striato-limbic regions, or any combination thereof. The electrode may contact at least a portion of the surface of the brain at the VC, SGC, amygdala, OFC, or striato-limbic regions, or other region of the brain suitable for detection. In some embodiments, the electrode may contact substantially the entire surface area at the VC, SGC, amygdala, OFC, or striato-limbic regions, or other region of the brain suitable for detection. In some embodiments, the electrode may additionally contact area(s) adjacent to the VC, SGC, amygdala, OFC, or striato-limbic regions. In some embodiments, an electrode array arranged on a planar support substrate may be used for detecting brain electrical signals for neural activity from one or more of the brain regions specified herein. The surface area of the electrode array may be determined by the desired area of contact between the electrode array and the brain. An electrode for implanting on a brain surface, such as, a surface electrode or a surface electrode array may be obtained from a commercial supplier. A commercially obtained electrode/electrode array may be modified to achieve a desired contact area. In some cases, the non-brain penetrating electrode (also referred to as a surface electrode) that may be used in the methods disclosed herein may be an electrocorticography (ECoG) electrode or an electroencephalography (EEG) electrode. In certain embodiments, a plurality of electrodes is positioned at one or more of the brain regions specified herein for detection of brain electrical signals by stereoelectroencephalography (sEEG).

In certain cases, placing the detection electrode at a target area or site (e.g., the VC, SGC, amygdala, OFC, or striato-limbic region, or other region of the brain suitable for detection) may involve positioning a brain penetrating electrode (also referred to as depth electrode) in the specified region(s) of the brain. For example, a detection electrode may be placed in the VC, SGC, amygdala, OFC, or striato-limbic region. In some embodiments, the detection electrode may additionally contact area(s) adjacent to the VC, SGC, amygdala, OFC, or striato-limbic region. In some embodiments, an electrode array may be used for detecting neural activity from the right VC, left VC, SGC, amygdala, OFC, or striato-limbic region as specified herein.

The depth to which a detection electrode is inserted into the brain may be determined by the desired level of contact between the electrode array and the brain. A brain-penetrating electrode array may be obtained from a commercial supplier. A commercially obtained electrode array may be modified to achieve a desired depth of insertion into the brain tissue.

The precise number of stimulation or detection electrodes contained in an electrode array (e.g., for electrical stimulation or detection of neural activity) may vary. In certain aspects, an electrode array may include two or more electrodes, such as 3 or more, including 4 or more, e.g., about 3 to 6 electrodes, about 6 to 12 electrodes, about 12 to 18 electrodes, about 18 to 24 electrodes, about 24 to 30 electrodes, about 30 to 48 electrodes, about 48 to 72 electrodes, about 72 to 96 electrodes, or about 96 or more electrodes. The electrodes may be arranged into a regular repeating pattern (e.g., a grid, such as a grid with about 1 cm spacing between electrodes), or no pattern. An electrode that conforms to the target site for optimal delivery of electrical stimulation may be used. One such example is a single multi contact electrode with eight contacts separated by 2½ mm. Each contract would have a span of approximately 2 mm. Another example is an electrode with two 1 cm contacts with a 2 mm intervening gap. Yet further, another example of an electrode that can be used in the present methods is a 2 or 3 branched electrode to cover the target site. Each one of these three-pronged electrodes has four 1-2 mm contacts with a center to center separation of 2 of 2.5 mm and a span of 1.5 mm.

The size of each electrode may also vary depending upon such factors as the number of electrodes in the array, the location of the electrodes, the material, the age of the patient, and other factors. In certain aspects, an electrode array has a size (e.g., a diameter) of about 5 mm or less, such as about 4 mm or less, including 4 mm-0.25 mm, 3 mm-0.25 mm, 2 mm-0.25 mm, 1 mm-0.25 mm, or about 3 mm, about 2 mm, about 1 mm, about 0.5 mm, or about 0.25 mm.

In certain embodiments, the method further comprises mapping subcortical and cortical stimulation responses of the subject to optimize positioning of an electrode for applying electrical stimulation. Positioning of a stimulation electrode is optimized to maximize clinical responses to electrical stimulation to relieve symptoms of a sleep-wake disorder such as excessive daytime sleepiness or lack of energy, excessive nighttime wakefulness, or inability to fall asleep or stay asleep long enough. In some embodiments, the VC, OFC, or SGC region, or other regions of the brain are mapped to determine optimal positioning of stimulation electrodes.

Assessment of the effectiveness of electrical stimulation at a particular site may be performed by measuring sleepiness or wakefulness of the subject using a visual-analog scale (VAS), a psychomotor vigilance test (PVT), a Likert scale, a Stanford Sleepiness Scale (SSS), a maintenance of wakefulness test (MWT), an Epworth sleepiness scale (ESS), a multiple sleep latency test (MSLT), or an Athens insomnia scale. Patients, who are treated with electrical stimulation, may be asked to self-rate their level of energy (e.g., from “not at all energetic” to “very energetic”) or their level of wakefulness/sleepiness (e.g., feeling active, vital, alert, or wide awake, or no longer fighting sleep, sleep onset soon, or having dream-like thoughts). Alternatively or additionally, a bedside observer may rate arousal metrics, e.g., using a 5-point Likert scale ranging from −2 (i.e., episodes of eye closure, decreased responsiveness to the environment, slowed speech rate) to 2 (i.e., high level of engagement with the environment, completion of tasks, relatively rapid speech rate).

In certain cases, the symptom that may be ameliorated by electrical stimulation is sleepiness. Sleepiness of a subject may be assessed using any standardized assessment method. In certain cases, sleepiness may be measured by using the Stanford Sleepiness Scale (SSS), Epworth sleepiness scale (ESS), or multiple sleep latency test (MSLT). Amelioration of sleepiness may include a reduction in the level of sleepiness of the subject compared to the level of sleepiness prior to the electrical stimulation.

In certain cases, the symptom that may be ameliorated by electrical stimulation is insomnia (e.g., excessive wakefulness, inability to fall sleep or stay asleep). Insomnia of a subject may be assessed using any standardized assessment method. In certain cases, insomnia may be measured by using the Likert scale, maintenance of wakefulness test (MWT), or Athens insomnia scale. Amelioration of insomnia may include an increase in the level of sleepiness of the subject compared to the level of sleepiness prior to the electrical stimulation.

In certain embodiments, the method further comprises mapping subcortical and cortical stimulation responses of the subject to optimize positioning of a detection electrode. Positioning of the detection electrode is optimized to detect brain activity features associated with excessive nighttime wakefulness or daytime sleepiness in need of treatment. For example, the levels of overall power, or power in specific frequency ranges (e.g., alpha, delta, beta, gamma, and/or high gamma) may be correlated with symptom severity and need for treatment with electrical stimulation. In some embodiments, the level of gamma frequency power (such as 30 Hz to 200 Hz) or beta frequency power (such as 13 Hz to 30 Hz) is correlated with symptom severity to determine if a patient is in need of treatment with electrical stimulation or if the amplitude of the electrical stimulation should be adjusted. Thus, detection electrodes may be positioned to optimize detection of brain activity in specific frequency ranges that correlate with symptom severity of a wake-sleep disorder.

In some embodiments, a relative spectral power level is measured for the gamma frequency neural oscillations, wherein higher relative power of the gamma frequency neural oscillations is associated with lower sleepiness and higher wakefulness compared to lower relative power of the gamma frequency neural oscillations. In some embodiments, the relative spectral power level is measured for low gamma frequency neural oscillations in a range from 31 Hz to 81 Hz or high gamma frequency neural oscillations in a range from 81 Hz to 150 Hz. Alternatively or additionally, coherence within certain spectral frequency bands or other features of network connectivity may be correlated with symptom severity and need for treatment with electrical stimulation.

In some embodiments, a relative spectral power level is measured for beta frequency neural oscillations, wherein higher relative power of the beta frequency neural oscillations is associated with wakefulness and insomnia. In some embodiments, the insomnia is caused by deep brain stimulation used for treatment of a disease. For example, insomnia may be caused by electrical stimulation to the anterior limb of the internal capsule (ALIC) in a subject being treated for obsessive-compulsive disorder or major depressive disorder. In some embodiments, a beta frequency brain electrical signal associated with wakefulness of the subject is recorded using a detection electrode, wherein the beta frequency brain electrical signal is from a striato-limbic region of the subject; and amplitude of the electrical stimulation is decreased during nighttime hours when spectral power of the beta frequency brain electrical signal exceeds a threshold level (see Example 2). In certain embodiments, the beta frequency brain electrical signal is in a range from 25 Hz to 30 Hz. In certain embodiments, electrical stimulation is applied at a frequency in a range of 0.5 Hz to 10 Hz to increase nighttime sleepiness of the subject when spectral power of the beta frequency brain electrical signal exceeds a threshold level.

Detection of brain activity may be performed by any method known in the art. For example, functional brain imaging of neural activity may be carried out by electrical methods such as electroencephalography (EEG), stereoelectroencephalography (SEEG), magnetoencephalography (MEG), single photon emission computed tomography (SPECT), as well as metabolic and blood flow studies such as functional magnetic resonance imaging (fMRI), and positron emission tomography (PET). In some embodiments, the VC region, SGC region, amygdala region, OFC, or striato-limbic region, or other regions are mapped to determine optimal positioning for detection electrodes. One or more of these regions may be implanted with detection electrodes to measure brain electrical signals from neural activity associated with a symptom (e.g., excessive daytime sleepiness or lack of energy, excessive nighttime wakefulness, inability to fall asleep or stay asleep long enough) of a sleep wake disorder.

As set forth here, the method involves applying electrical stimulation in a manner effective to treat a sleep-wake disorder and/or a symptom (e.g., excessive daytime sleepiness or lack of energy, excessive nighttime wakefulness, inability to fall asleep or stay asleep long enough) of a sleep-wake disorder in a subject when neural activity associated with a symptom of the sleep-wake disorder is detected. Closed-loop therapy can be performed with a neurostimulator that records brain electrical signals from neural activity associated with a symptom of the sleep-wake disorder and delivers electrical stimulation when the level of a brain electrical signal that is detected exceeds a set threshold level indicating a level of symptom severity in need of treatment. The parameters for applying the electrical stimulation may be determined empirically during treatment or may be pre-defined, such as, from a clinical study. The parameters of the electrical stimulation may include one or more of frequency, pulse width/duration, duty cycle, intensity/amplitude, pulse pattern, program duration, program frequency, and the like.

Frequency refers to the pulses produced per second during stimulation and is stated in units of Hertz (Hz, e.g., 60 Hz=60 pulses per second). The frequencies of electrical stimulation used in the present methods may vary widely depending on numerous factors and may be determined empirically during treatment of the subject or may be pre-defined. In certain embodiments, the electrical stimulation is applied at a frequency in a range from 70 Hz to 250 Hz, including any frequency with this range such as 70 Hz, 75 Hz, 80 Hz, 85 Hz, 90 Hz, 95 Hz, 100 Hz, 105 Hz, 110 Hz, 115 Hz, 120 Hz, 125 Hz, 130 Hz, 135 Hz, 140 Hz, 145 Hz, 150 Hz, 155 Hz, 160 Hz, 165 Hz, 170 Hz, 175 Hz, 180 Hz, 185 Hz, 190 Hz, 195 Hz, 200 Hz, 205 Hz, 210 Hz, 215 Hz, 220 Hz, 225 Hz, 230 Hz, 235 Hz, 240 Hz, 245 Hz, or 250 Hz, wherein the level of wakefulness or energy of the subject is increased and/or reaction time of the subject is decreased compared to the level of wakefulness or energy or reaction time of the subject before applying the electrical stimulation. In other embodiments, the electrical stimulation is applied at a frequency in a range from 0.5 Hz to 10 Hz, including any frequency within this range such as 0.5 Hz, 0.6 Hz, 0.7 Hz, 0.8 Hz, 0.9 Hz, 1.0 Hz, 1.2 Hz, 1.4 Hz, 1.6 Hz, 1.8 Hz, 2.0 Hz, 2.2 Hz, 2.4 Hz, 2.6 Hz, 2.8 Hz, 3.0 Hz, 3.2 Hz, 3.4 Hz, 3.6 Hz, 3.8 Hz. 4.0 Hz, 4.2 Hz, 4.4 Hz, 4.6 Hz, 4.8 Hz, 5.0 Hz, 5.2 Hz, 5.4 Hz, 5.6 Hz, 5.8 Hz, 6.0 Hz, 6.2 Hz, 6.4 Hz, 6.6 Hz, 6.8 Hz, 7.0 Hz, 7.2 Hz, 7.4 Hz, 7.6 Hz, 7.8 Hz, 8.0 Hz, 8.2 Hz, 8.4 Hz, 8.6 Hz, 8.8 Hz, 9.0 Hz, 9.2 Hz, 9.4 Hz, 9.6 Hz, 9.8 Hz, or 10 Hz, wherein the level of sleepiness and/or reaction time of the subject is increased compared to the level of sleepiness of the subject before applying the electrical stimulation. In certain embodiments, the amplitude of current is in a range from 0.1 mA to 30 mA, such as, 0.1 mA-25 mA, such as, 0.1 mA-20 mA, 0.1 mA-15 mA, 0.1 mA-10 mA, 1 mA-20 mA, 1 mA-10 mA, 2 mA-30 mA, 2 mA-15 mA, or 2 mA-10 mA.

The electrical stimulation may be applied in pulses such as a uniphasic or a biphasic pulse. The time span of a single pulse is referred to as the pulse width or pulse duration. The pulse width used in the present methods may vary widely depending on numerous factors (e.g., severity of the symptoms, status of the patient, and the like) and may be determined empirically or may be pre-defined. In certain embodiments, the method may involve applying an electrical stimulation at a pulse width of about 10 μsec-990 μsec, for example, 30 μsec-990 μsec, 50 μsec-990 μsec, 75 μsec-990 μsec, 100 μsec-990 μsec, 200 μsec-990 μsec, 300 μsec-990 μsec, 500 μsec-990 μsec, 500 μsec-900 μsec, 30 μsec-900 μsec, 50 μsec-900 μsec, 75 μsec-900 μsec, 100 μsec-900 μsec, 200 μsec-900 μsec, 300 μsec-900 μsec, 500 μsec-900 μsec, 30 μsec-500 μsec, 50 μsec-450 μsec, 75 μsec-300 μsec, 100 μsec-200 μsec, or 100 μsec-550 μsec. In some embodiments, the electrical stimulation is applied at a pulse width of about 100 μsec to about 140 μsec, including any pulse width within this range such as 100, 102, 104, 106, 108, 110, 112, 114, 116, 118, 120, 122, 124, 126, 128, 130, 132, 134, 136, 138, or 140 μsec.

The electrical stimulation may be applied for a stimulation period of 0.1 sec-1 month, with periods of rest (i.e., no electrical stimulation) possible in between. In certain cases, the period of electrical stimulation may be 0.1 sec-1 week, 1 sec-1 day, 10 sec-12 hours, 1 min-6 hours, 10 min-1 hour, and so forth. In certain cases, the period of electrical stimulation may be 1 sec-1 min, 1 sec-30 sec, 1 sec-15 sec, 1 sec-10 sec, 1 sec-6 sec, 1 sec-3 sec, 1 sec-2 sec, or 6 sec-10 sec. The period of rest in between each stimulation period may be 60 sec or less, 30 sec or less, 20 sec or less, or 10 sec.

In some embodiments, the rest period ranges from 10 seconds to 10 minutes. For example, electrical stimulation may be applied intermittently with a stimulation period of 6 seconds and a rest period of 10 seconds. In another example, electrical stimulation is applied intermittently with a stimulation period of 30 seconds and a rest period of 2 minutes.

For a subject who has hypersomnia, a nighttime rest period without electrical stimulation may be employed to avoid interfering with sleep when the subject wishes to sleep at night, and a daytime stimulation period may be employed, wherein electrical stimulation is used to improve wakefulness and energy during the day. Conversely, for a subject who has insomnia, a nighttime stimulation period may be employed, wherein electrical stimulation is used to promote sleepiness to allow a subject to sleep when the subject wishes to sleep at night, and a daytime rest period may be employed when such stimulation is unnecessary. In some cases, the frequency of stimulation may be adjusted depending on the sleep-wake levels of the subject, wherein different frequencies of stimulation may be used during different time periods as needed. For example, electrical stimulation may be applied at a frequency in a range from 70 Hz to 250 Hz to increase daytime wakefulness or energy and/or decrease reaction time of the subject when needed, and electrical stimulation may be applied at a frequency in a range of 0.5 Hz to 10 Hz to increase nighttime sleepiness of the subject when needed.

The electrical stimulation may be applied with an amplitude of current of 0.1 mA-30 mA, such as, 0.1 mA-25 mA, such as, 0.1 mA-20 mA, 0.1 mA-15 mA, 0.1 mA-10 mA, 0.1 mA-2 mA, 0.1 mA-1 mA, 1 mA-20 mA, 1 mA-10 mA, 2 mA-30 mA, 2 mA-15 mA, 2 mA-10 mA, or 1 mA-2 mA. In some embodiments, the amplitude of current is 0.1 mA-1.5 mA, or any amplitude of current in this range such as 0.1, 0.2, 0.3, 0.4, 0.5, 0.6, 0.7, 0.8, 0.9, 1.0, 1.1, 1.2, 1.3, 1.4, or 1.5 mA.

The electrical stimulation having the parameters as set forth above may be applied over a program duration of around 1 day or less, such as, 18 hours, 6 hours, 3 hours, 1 hour, 45 minutes, 30 minutes, 20 minutes, 10 minutes, or 5 minutes, or less, e.g., 1 minute-5 minutes, 2 minutes-10 minutes, 2 minutes-20 minutes, 2 minutes-30 minutes, 5 minutes-10 minutes, 5 minutes-30 minutes, or 5 minutes-15 minutes which period would include the application of pulses and the intervening rest period. The program may be repeated at a desired program frequency till treatment of the subject is achieved. As such, a treatment regimen may include a program for electrical stimulation at a desired program frequency and program duration. In some embodiments, the treatment regimen is controlled by a control unit in communication with a pulse generator connected to the one or more stimulation electrodes in a closed-loop treatment regimen.

In some embodiments, a cap on the maximum number of electrical stimulations per day can be set. For example, the maximum number of electrical stimulations per day may range from 50 therapies per day to 500 therapies per day, including any number of therapies per day in this range such as 50, 75, 100, 125, 150, 175, 200, 225, 250, 275, 300, 325, 350, 375, 400, 425, 450, 475, or 500 therapies per day. Alternatively or additionally a cap can be set on the total amount of time of electrical stimulation per day. For example, the total amount of time of electrical stimulation per day may range from 10 minutes to 100 minutes of total stimulation per day, including any amount of time within this range such as 10, 20, 30, 40, 50, 60, 70, 80, 90, or 100 minutes of total stimulation time per day.

In certain cases, effectiveness of treatment may be assessed by detecting activity (e.g., electrical signals) from a secondary region of the brain, which may be within the VC, SGC, amygdala, or OFC, or another area. For example, the secondary region(s) may be one or more of the VC, SGC, amygdala, or OFC. Detection of brain activity at a secondary region of the brain may be performed by functional brain imaging. Functional brain imaging may be carried out by electrical methods such as electroencephalography (EEG), magnetoencephalography (MEG), single photon emission computed tomography (SPECT), as well as metabolic and blood flow studies such as functional magnetic resonance imaging (fMRI), and positron emission tomography (PET). In some embodiments, electrical methods for assessing effectiveness of treatment may involve use of a detection electrode as described herein or placement of an additional electrode for measuring electrical signals at a secondary region of the brain. One or more regions of the brain may be implanted with an electrode and electrical signals measured for assessment of effectiveness of the treatment. Any suitable electrodes may be used for measurements and may include one or more surface electrodes (non-brain penetrating electrode(s)) or one or more depth electrodes (brain penetrating electrode(s)) as described herein.

Assessment of effectiveness of treatment and assessment of amelioration of a symptom of a sleep-wake disorder may be performed at any suitable time point after commencement of the treatment procedure, for example, during closed-loop therapy or after a treatment regimen is complete. Embodiments of the subject methods include assessing effectiveness of treatment or amelioration of a symptom of the sleep-wake disorder within seconds, minutes, hours, or days after the initial treatment regimen has been completed. In some instances, assessment may be performed at multiple time points. In some cases, more than one type of assessment may be performed at the different time points. In some embodiments, a subject's brain activity (e.g., at one or more secondary regions) may be measured prior to the application of electrical stimulation, and assessing may include comparing the subject's brain activity at one or more secondary regions after the treatment to that before the treatment and a change in the post-treatment brain activity may indicate successful treatment.

In some embodiments, the subject is monitored for a period of time during closed-loop therapy using a detection electrode, wherein increasing numbers of the brain electrical signals exceeding the threshold level detected by the detection electrode within a later recording period compared to an earlier recording period indicate increasing severity of a symptom of the sleep-wake disorder; and decreasing numbers of brain electrical signals exceeding the threshold level detected by the detection electrode within a later recording period compared to an earlier recording period indicate decreasing severity of the symptom of the sleep-wake disorder.

The methods and systems provided herein may be used to ameliorate symptoms of a sleep-wake disorder such as excessive daytime sleepiness or lack of energy, excessive nighttime wakefulness, or inability to fall asleep or stay asleep long enough. Upon completion of a treatment regimen, the patient may be assessed for effectiveness of the treatment and the treatment regimen may be repeated, if needed. In certain cases, the treatment regimen may be altered before repeating. For example, one or more of the frequency, pulse width, current amplitude, period of electrical stimulation, program duration, program frequency, and/or placement of stimulation or detection electrodes may be altered before starting a second treatment regimen.

In certain cases, the treatment regimen may be tailored to the desired outcome. For example, for a patient suffering from an acute form of a sleep-wake disorder, the treatment regimen may be chosen to provide an acute alleviation of one or more symptoms of the disorder. In contrast, the treatment regimen for a patient suffering chronically from a sleep-wake disorder may be tailored for a chronic relief of one or more symptoms of the disorder. A treatment regimen for chronic relief of one or more symptoms of the disorder may also delay reappearance of symptoms of the disorder and may even prevent the symptoms from appearing. In certain cases, a treatment regimen that is a combination of treatment regimens for acute and chronic alleviation of one or more symptoms of the disorder may be employed.

Application of the method may include a prior step of selecting a patient for treatment based on need as determined by clinical assessment, which may include cognitive assessment, anatomical assessment, behavioral assessment and/or neurophysiological assessment. In certain cases, a subject may be selected for treatment if the subject is suffering from a sleep-wake disorder. For example, a patient may be selected for treatment if the patient is experiencing hypersomnia or insomnia that is severe enough to interfere with normal day-time functioning. Such a patient may be implanted with stimulation and/or detection electrodes.

In certain aspects, the methods and systems of the present disclosure may include measurement of brain activity, for example, electrical activity in the amygdala, where the level of gamma-frequency power may be measured. In certain cases, electrical activity from a plurality of locations in the amygdala may be measured and averaged. In some embodiments, electrical activity in the gamma frequency range (such as 30 Hz to 200 Hz) may be measured from the right amygdala, left amygdala, right orbitofrontal cortex, left orbitofrontal cortex, right subgenual cingulate, left subgenual cingulate, right VC, or left VC, or any combination thereof. In some cases, electrical activity in one or more locations in the brain may be measured during a period extending from prior to stimulation to the period during which stimulation to the brain is applied, or to a period after stimulation to the brain has been applied, and monitored for a decrease in the power of gamma-frequency (such as 30 Hz to 200 Hz) activity and/or increase in cortical excitability. In some cases, when decreased power of gamma-frequency (such as 30 Hz to 200 Hz) activity and/or increased cortical excitability is within a normal range (e.g., a range associated with a substantial lack of the disorder), the methods and systems do not apply a further stimulation to the brain. Alternatively, when decreased power of gamma-frequency (such as 30 Hz to 200 Hz) activity and/or increased cortical excitability is not within a normal range (e.g., a range associated with a substantial lack of the disorder), the methods and systems may apply a further stimulation to the brain. In certain cases, the application of electrical stimulation to the brain may suppress gamma-frequency power across the right amygdala, left amygdala, right orbitofrontal cortex, left orbitofrontal cortex, left subgenual cingulate, right subgenual cingulate, right VC, or left VC. The decrease may be as compared to the power prior to the application of stimulation. In certain cases, the application of electrical stimulation to the brain may alter other neural features from one more regions of the brain. The alterations may be compared to the state of these features prior to the application of stimulation.

In some embodiments, a relative spectral power level is measured for the gamma frequency neural oscillations, wherein higher relative power of the gamma frequency neural oscillations is associated with lower sleepiness and higher wakefulness compared to lower relative power of the gamma frequency neural oscillations. In some embodiments, the relative spectral power level is measured for low gamma frequency neural oscillations in a range from 31 Hz to 81 Hz. or high gamma frequency neural oscillations in a range from 81 Hz to 150 Hz.

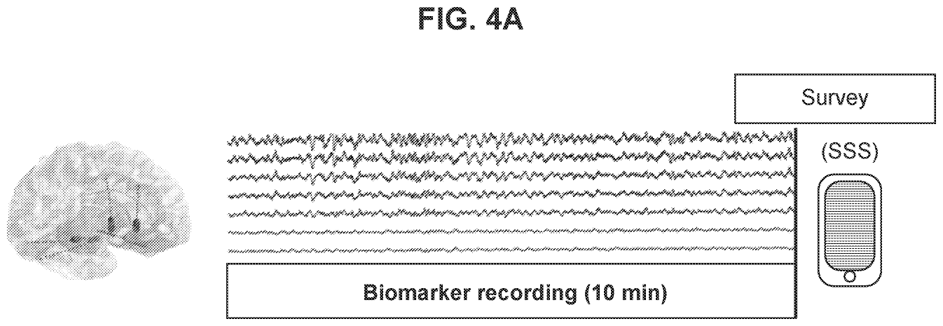

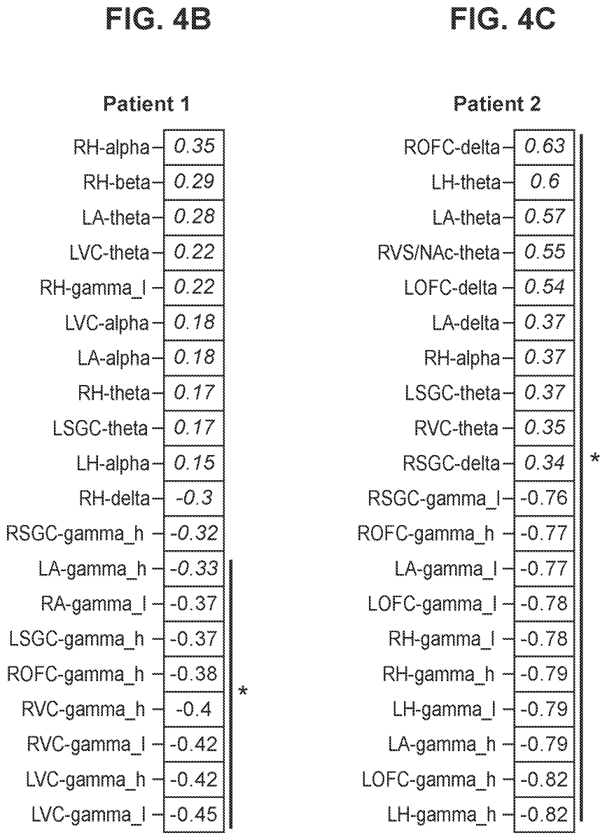

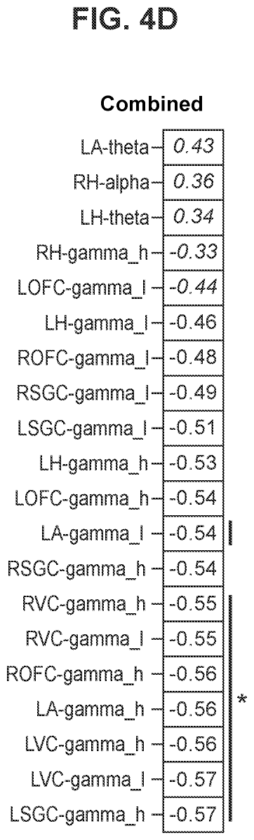

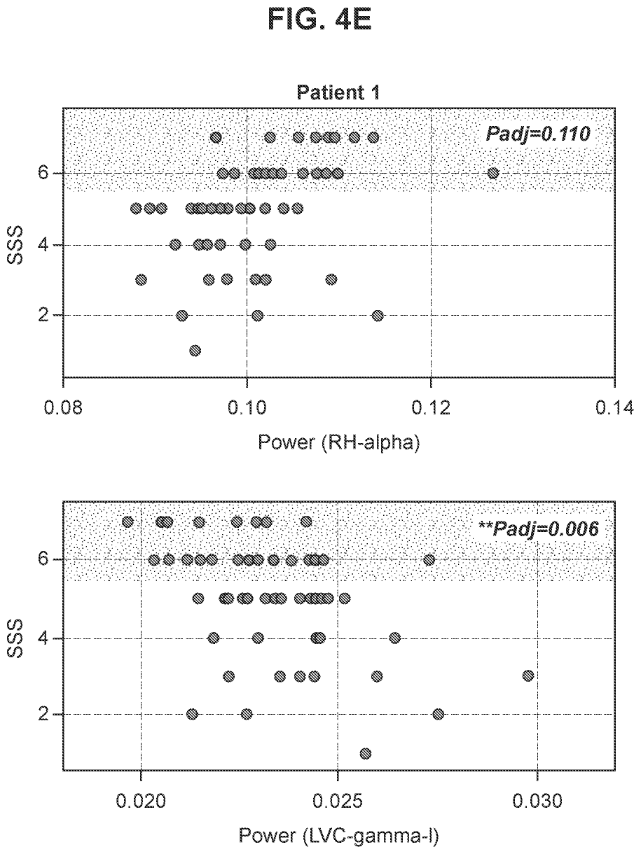

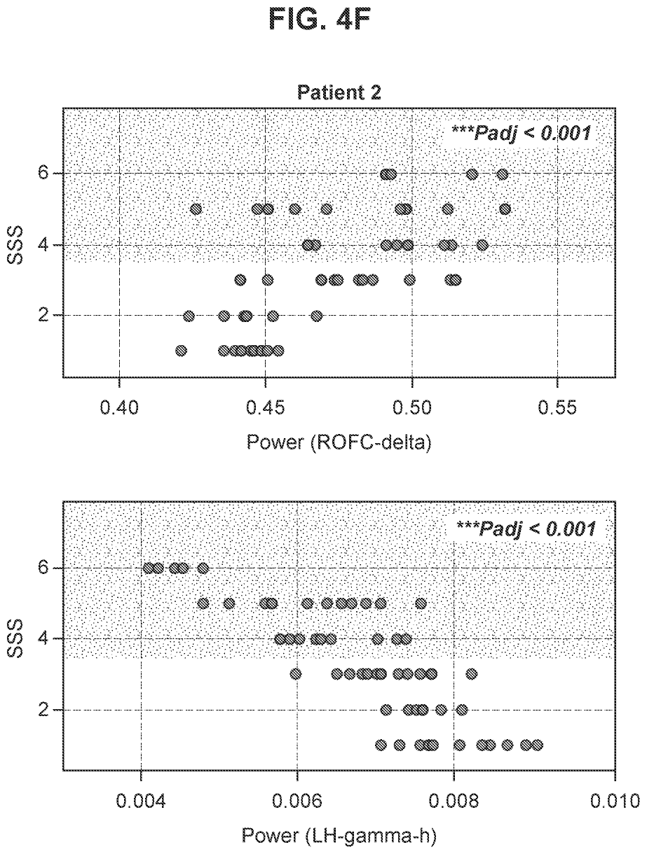

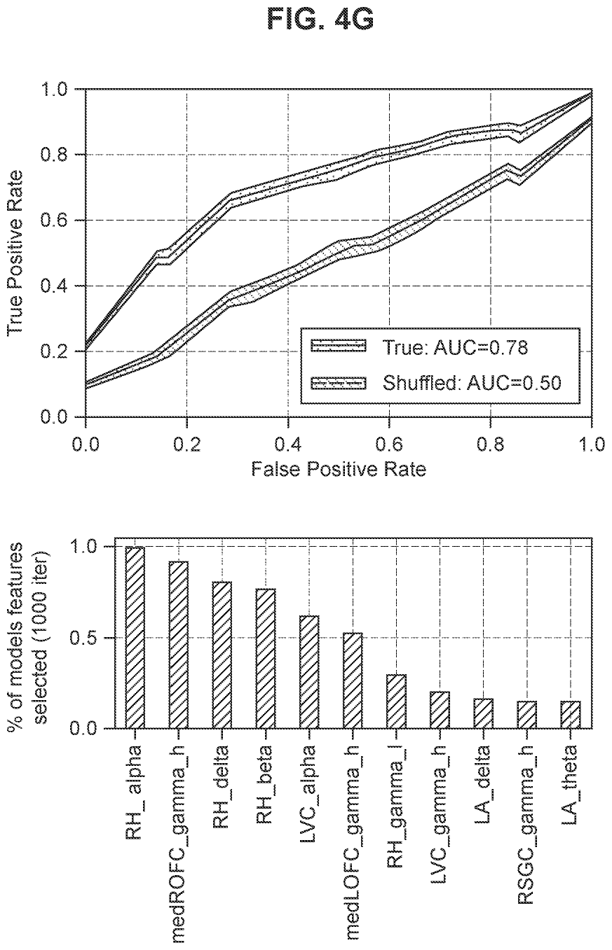

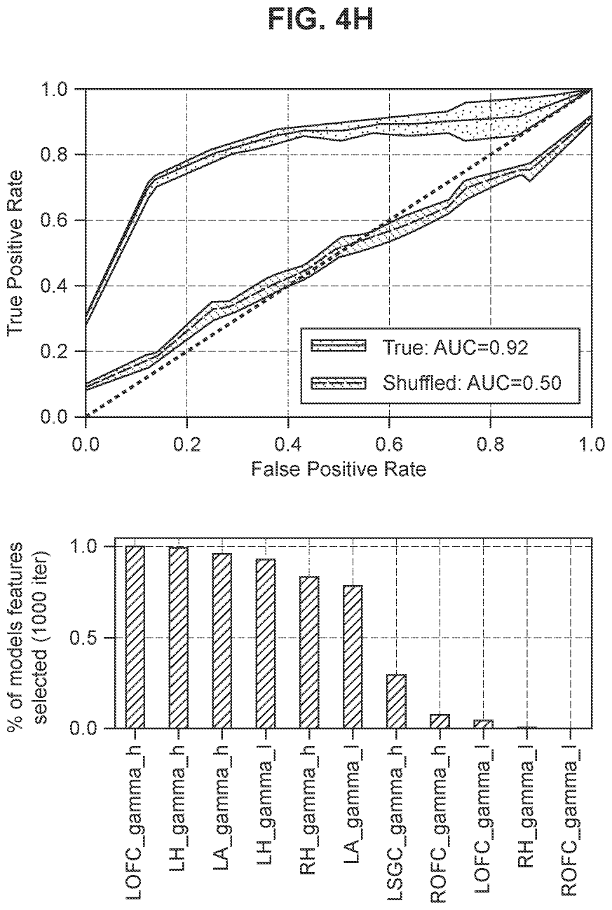

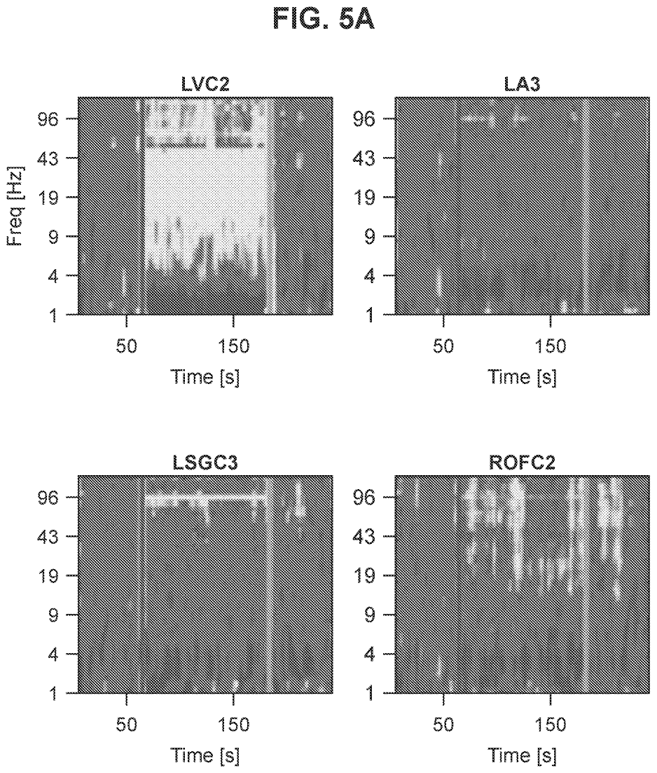

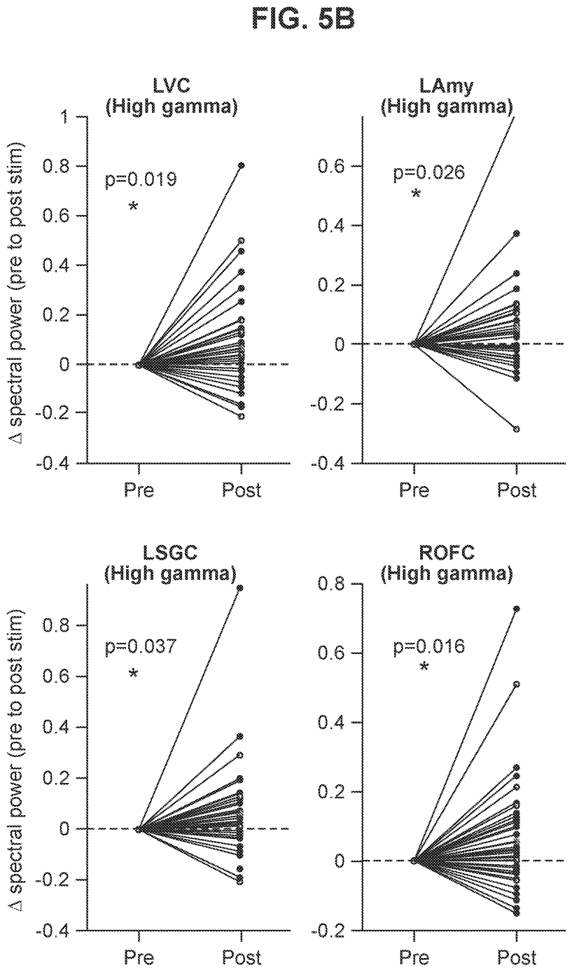

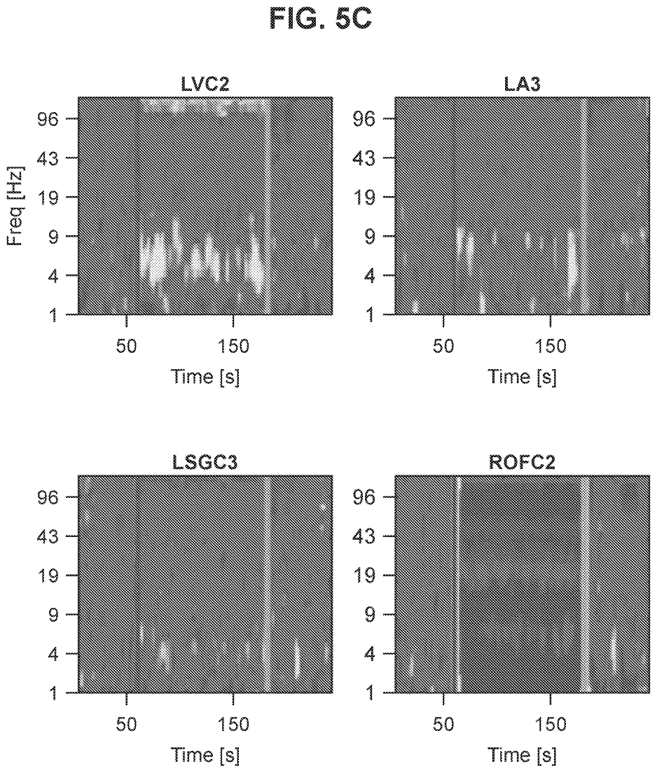

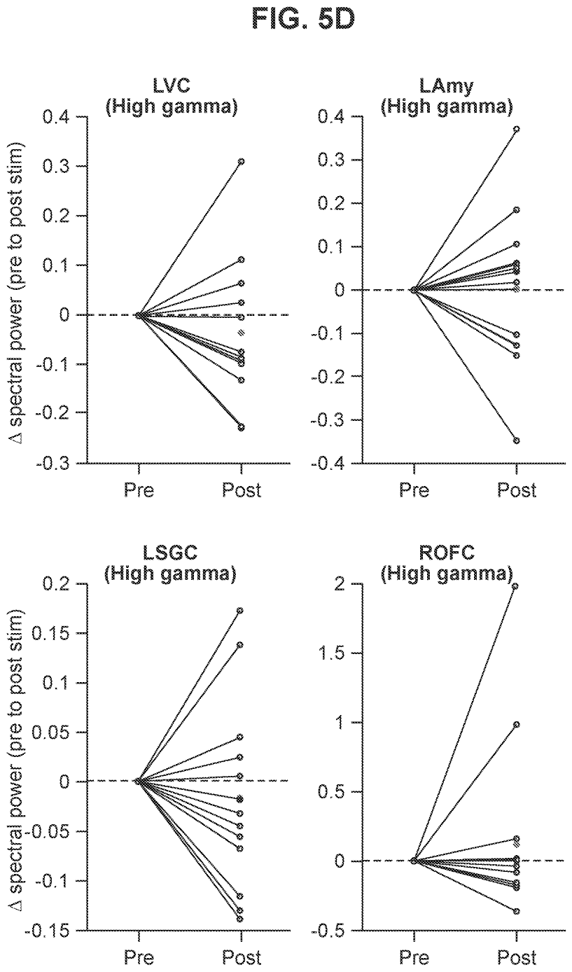

A closed-loop method allows determination of parameters of electrical stimulation based upon real-time feedback signals from the brain of the subject. Closed-loop methods and systems allow for automation of treatment of the subject including real-time need-based modulation of the treatment regimen. Exemplary closed-loop methods and associated systems for treatment of a sleep-wake disorder are further discussed in the Examples section and are depicted in FIGS. 4 and 5. Closed-loop methods and systems for automated delivery of electrical stimulation are further described below.

### Closed-Loop Method for Automated Delivery of Electrical Stimulation

In certain embodiments, a control algorithm is used to automate the delivery of electrical stimulation in response to detection of neural activity associated with a symptom of a sleep-wake disorder. According to certain embodiments, the method may include receiving an electrical signal associated with wakefulness or sleepiness from a region of the brain of the subject via a detection electrode; applying electrical signal metrics to a control algorithm that is tuned to a clinically relevant target (e.g., a range of signal indicative of effective treatment and/or a range of signal indicative of severity of symptoms and the need for treatment); automatically delivering electrical stimulation to the brain via the stimulation electrode in a manner effective to treat the sleep-wake disorder or ameliorate a symptom of the sleep-wake disorder if the electrical signal metrics indicate that the patient is in need of treatment. For example, electrical activity in the gamma frequency range (such as 30 Hz to 200 Hz) or beta frequency range (such as 13 Hz to 30 Hz) from the ventral capsule (VC) region, subgenual cingulate (SGC) region, amygdala region, orbitofrontal cortex (OFC) region, or striato-limbic region may be measured with a detection electrode, wherein the control algorithm receives the electrical activity data from the detection electrode and automates delivery of electrical stimulation via a stimulation electrode to a VC region (e.g., a right VC region and/or a left VC region), OFC region, or SGC region or any combination thereof when the level of gamma frequency power (such as 30 Hz to 200 Hz) or beta frequency power (such as 13 Hz to 30 Hz) is in a certain range indicating that a patient has a symptom of a sleep-wake disorder severe enough that the patient is in need of treatment. In some embodiments, the gamma frequency neural oscillations are low gamma frequency neural oscillations in a range from 31 Hz to 81 Hz or high gamma frequency neural oscillations in a range from 81 Hz to 150 Hz. In some embodiments, the method further comprises measuring a relative spectral power level for the gamma frequency neural oscillations or beta frequency neural oscillations, wherein higher relative power of the gamma frequency neural oscillations or beta frequency neural oscillations is associated with lower sleepiness and higher wakefulness compared to lower relative power of the gamma frequency neural oscillations or beta frequency neural oscillations. In some embodiments, one or more programmed stimulation parameters are modulated according to the algorithm's control law based on the recorded electrical activity data; and modulated electrical stimulation is delivered to the VC region (e.g., a right VC region and/or a left VC region), OFC region, or SGC region or any combination thereof via the stimulation electrode in a manner effective to ameliorate the symptom of the sleep-wake disorder.

In certain embodiments, electrical stimulation is applied when a recorded brain electrical signal associated with wakefulness or sleepiness of the subject exceeds a threshold level. For treating hypersomnia, electrical stimulation at a frequency in a range from 70 Hz to 250 Hz may be automatically delivered to a VC region (e.g., a right VC region and/or a left VC region), OFC region, or SGC region or any combination thereof via the stimulation electrode in a manner effective to increase wakefulness or energy of the subject when a brain electrical signal associated with sleepiness from a VC region, a SGC region, an amygdala region, or an OFC region of the subject exceeds a threshold level. For treating insomnia, electrical stimulation at a frequency in a range from 0.5 Hz to 10 Hz, may be automatically delivered to a VC region (e.g., a right VC region and/or a left VC region), OFC region, or SGC region or any combination thereof via the stimulation electrode in a manner effective to increase sleepiness of the subject when a brain electrical signal associated with wakefulness from a VC region, a SGC region, an amygdala region, an OFC region, or a striato-limbic region of the subject exceeds a threshold level.

In addition, time periods for electrical stimulation and rest periods free of electrical stimulation may be set for the subject. For example, for a subject who has hypersomnia, a nighttime rest period without electrical stimulation may be employed to avoid interfering with sleep when the subject wishes to sleep at night, and a daytime stimulation period may be employed, wherein electrical stimulation is used to improve wakefulness and energy durng the day. Conversely, for a subject who has insomnia, a nighttime stimulation period may be employed, wherein electrical stimulation is used to promote sleepiness to allow a subject to sleep when the subject wishes to sleep at night, and a daytime rest period may be employed when such stimulation is unnecessary. In some cases, the frequency of stimulation may be adjusted depending on the sleep-wake levels of the subject, wherein different frequencies of stimulation may be used during different time periods as needed. For example, electrical stimulation may be applied at a frequency in a range from 70 Hz to 250 Hz to increase daytime wakefulness and/or energy and/or decrease reaction time of the subject when needed, and electrical stimulation may be applied at a frequency in a range of 0.5 Hz to 10 Hz to increase nighttime sleepiness of the subject when needed.

As described in the foregoing sections, effectiveness of treatment or amelioration of a symptom of the sleep-wake disorder may be assessed by detecting electrical activity from the VC, SGC, amygdala, OFC, or striato-limbic region(s) of the brain using a detection electrode. In an open-loop system, stimulation is delivered in a pre-programmed way or manually by a user but is not automatically controlled by real-time neural feedback from the patient's brain. The electrical activity may be analyzed by a computing means which may output recommendations based on comparing the electrical activity to a predetermined range. A user may then carry out the recommendations, such as, changing a parameter of the electrical stimulation program prior to starting another treatment regimen. In a closed-loop system, by contrast, a computing means can automatically update stimulation parameters based upon analysis of the recorded electrical signal and/or automatically deliver stimulation according to the electrical stimulation program. In some embodiments, either an open-loop or a closed-loop system may be integrated with a mechanism for user intervention, for example by allowing user-override of open-loop or closed-loop stimulation programs to enact or prevent stimulation that would ordinarily occur, or to manually change parameters of such stimulation.

In some embodiments, the computing means for directing closed-loop stimulation may be a combination of hardware/software which may be connected wirelessly or by wire to the measurement electrodes. The computing means may communicate with a control unit (also referred to as a control module) that controls a pulse generator connected to the stimulation electrodes. In certain embodiments, the computing means may be connected to a recorder (e.g., a neurophysiological recorder) that records brain activity measured by the detection electrodes. The computing means may include a control algorithm that determines modification of stimulation parameters based on real-time outputs of the neurophysiological recorder. The algorithm may operate by simple on/off control of stimulation at set parameters, modifying only the on/off parameter with each evaluation cycle, or may determine sophisticated modification of a range of stimulation parameters with each cycle. In some cases, the algorithm may be based on information related to the sleep-wake disorder, such as, a range of electrical activity that is indicative of the presence of the sleep-wake disorder or severity of symptoms. The algorithm may also include additional information such as a brain activity profile of a normal subject (not suffering from a sleep-wake disorder). Regardless of the particular control algorithm structure, the computing means may be tuned to a clinically relevant target (e.g., a range of signal indicative of effective treatment and/or a range of signal indicative of severity of symptoms and the need for treatment) that directs modulation of one or more programmed stimulation parameters according to the algorithm's control law, applying the modulated electrical stimulation (e.g., to the VC, SGC, or OFC region, or other region) via the stimulation electrode.

In some cases, the computing means, via a control algorithm, may determine whether the received electrical signals are within or outside a predetermined range of neural signals indicative of the presence of the sleep-wake disorder. When the received electrical signals are outside this predetermined range, then the computing means determines that the sleep-wake disorder has been treated and/or a symptom of the sleep-wake disorder (e.g., excessive daytime sleepiness or lack of energy, excessive nighttime wakefulness, inability to fall asleep or stay asleep long enough) has been ameliorated. The computing means may then communicate with the control unit to direct stimulation shut-off by the pulse generator. When the received electrical signals are within the predetermined range of neural signals indicative of the presence of the sleep-wake disorder, then the computing means determines that the sleep-wake disorder is in need of treatment and/or a symptom of the sleep-wake disorder (e.g., excessive daytime sleepiness or lack of energy, excessive nighttime wakefulness, inability to fall asleep or stay asleep long enough) is severe enough to require treatment. The control algorithm within the computing means may then determine whether the initial step of applying electrical stimulation should be repeated and/or whether a parameter of the electrical stimulation should be modified prior to the step of applying electrical stimulation. The computing means, via the control unit, may then communicate with the control unit to provide the appropriate instructions to the pulse generator.

In some embodiments, the computing means may determine whether the received electrical signals are within or outside a second predetermined range, where the second predetermined range is indicative of treatment of the sleep-wake disorder and/or amelioration of a symptom of the sleep-wake disorder. When the received electrical signals are within the second predetermined range, then the computing means determines that the sleep-wake disorder has been treated and/or a symptom of the sleep-wake disorder (e.g., excessive daytime sleepiness or lack of energy, excessive nighttime wakefulness, inability to fall asleep or stay asleep long enough) has been ameliorated. The computing means may then communicate with the control unit to direct stimulation switch-off by the pulse generator. When the received electrical signals are outside the second predetermined range, then the computing means determines that the sleep-wake disorder has not been treated and/or a symptom of the sleep-wake disorder (e.g., excessive daytime sleepiness or lack of energy, excessive nighttime wakefulness, inability to fall asleep or stay asleep long enough) has not been ameliorated. The control algorithm within the computing means may then determine whether the initial step of applying electrical stimulation should be repeated and/or whether a parameter of the electrical stimulation modified prior to the step of applying electrical stimulation. The processor may then communicate with the control unit to provide the appropriate instructions to the pulse generator.

Thus, in certain aspects, the subject methods operate as a closed-loop control system which may automatically adjust one or more parameters in response to electrical activity from a region of the brain of a subject and/or automatically deliver stimulation according to the electrical stimulation program. In some embodiments, the closed-loop control system automatically delivers stimulation according to set parameters when the received electrical signals are within a predetermined range indicative of the need for treatment. Exemplary closed-loop methods and associated systems are described in the Examples section of the application and are illustrated in FIGS. 4 and 5.

In some aspects, the closed loop system may be used to sense a subject's need for treatment using the methods disclosed herein. For example, the closed loop system may be programmed to monitor brain activity from one or more regions of the brain and compare the brain activity to a range indicative of the sleep-wake disorder or a symptom of the sleep-wake disorder. Upon detection of electrical activity indicative of the sleep-wake disorder or a symptom of the sleep-wake disorder, the closed loop system may automatically commence a treatment protocol of applying electrical stimulation.

In additional aspects, the closed loop system may be used as a system for monitoring brain activity and correlating the brain activity to the subject's level of sleepiness or wakefulness. For example, since the closed loop system is configured for recording electrical signals from a subject's brain, the subject's level of sleepiness or wakefulness may be monitored in real-time and correlated to the measured electrical signals to provide a biomarker that is related to the subject's level of sleepiness or wakefulness. For example, electrical activity measured for a subject who is experiencing hypersomnia can be used to develop a biomarker, e.g., a range of electrical activity indicative of excessive daytime sleepiness. Electrical activity measured for a subject who is experiencing insomnia can be used to develop a biomarker, e.g., a range of electrical activity indicative of excessive nighttime wakefulness, and so on. As such, closed loop systems are useful for prognosis as well as diagnosis of sleep-wake disorders.

It is understood that electrical signals that are indicative of excessive daytime sleepiness or nighttime wakefulness of a subject may be recorded from a subject's brain and may be used in aspects outside of a closed loop system. For example, electrical signals indicative of excessive daytime sleepiness or nighttime wakefulness of a subject may be recorded from the VC, SGC, amygdala, or OFC, or other region using electrodes or another device operably coupled to the patient's brain which electrodes or device may or may not be part of a closed loop system. The patient may be treated as disclosed herein (e.g., by applying electrical stimulation to the VC, SGC, or OFC region, or other region), and electrical signals recorded from the same region in real time as the treatment is administered or after the treatment is administered. The electric signals recorded after the administration of electrical stimulation is commenced may then be compared to the electric signals recorded prior to the treatment to determine features in the recorded electric signals that change post-treatment. These features provide a feedback signal to indicate whether the treatment is having an effect on the patient's level of sleepiness or wakefulness. These features can also serve as feedback signals to a closed loop system. These features may include the overall power, or power in specific frequency ranges (e.g., alpha, delta, beta, gamma, and/or high gamma). In some cases, these features may be patient specific or specific to a particular sleep-wake disorder, or both. For example, some of the features may be features found in a plurality of patients having a particular sleep-wake disorder (e.g., hypersomnia or insomnia); some of the features may be features in a particular patient which may not be found in a significant number of other patients having the same sleep-disorder. In some embodiments, a combination of patient-specific features and sleep-wake disorder specific features may be monitored to assess efficacy of treatment.

In a particular aspect, the closed loop system and methods provided herein may involve a recording of electrical signals from one or more regions (e.g., right amygdala, left amygdala, right OFC, left OFC, right SGC, left SGC, right VC, or left VC) of a patient's brain, where the patient has a sleep-wake disorder. The patient may then be treated by application of electrical stimulation to the VC, SGC, or OFC region, or other region of the brain, and electrical signals may be recorded from the same regions of the brain (e.g., VC, SGC, amygdala, or OFC) and compared to the pre-treatment recording. Features in the recorded signals that changed after the treatment would correspond to biomarkers that indicate whether the treatment is having an effect. The change in recorded signals can also optionally be correlated to sleep-wake levels reported by the patient after the treatment. The change can be used for modulating the treatment in a closed loop system. For example, when the change in the recorded signal correlates with an improvement in the patient's daytime wakefulness or energy or nighttime sleep, those features would indicate to a computing means of a closed loop system that further treatment need not be performed.

In certain aspects, methods of the present disclosure that may be embodied in an open- or a closed-loop system may include measuring brain activity from a subject having or suspected or having a sleep-wake disorder, such as, hypersomnia or insomnia, where when the subject has a level of gamma range frequencies (e.g., 30 Hz to 200 Hz) that is higher than a normal range (e.g., range reflective of a normal brain, such as, brain of a person not suffering from the sleep-wake disorder) or higher than a threshold level (e.g., indicating a symptom severe enough that treatment is needed), the subject is treated by applying electrical stimulation to the VC, SGC, or OFC region, or other region via an electrode positioned at or adjacent to the VC, SGC, or OFC region, or other region in a manner effective to reduce the level of gamma range frequencies in the brain of the subject. The brain activity may be measured at one or more brain regions (e.g., VC, SGC, amygdala, or OFC, or any combination thereof). In some aspects, the system may monitor brain activity such as levels of gamma frequency activity, and if the level of gamma frequency activity is higher than a reference range (e.g., range reflective of a normal brain, such as, brain of a person not suffering from the disorder) and/or higher than a threshold level (e.g., indicating a symptom severe enough that treatment is needed), the closed loop system may apply electrical stimulation to the VC, SGC, or OFC region, or other region via an electrode positioned at or adjacent to the VC, SGC, or OFC region, or other region in a manner effective to reduce the level of gamma frequency activity (e.g., 30 Hz to 200 Hz) in the brain of the subject.

In some embodiments, one or more pattern recognition methods can be used in analyzing recorded brain electrical activity data to automate detection of brain activity features that distinguish symptom states of a sleep-wake disorder that are severe enough to need treatment from symptomless states (baseline) or minor symptom states that do not need treatment. The models and/or algorithms can be provided in machine readable format and may be used to correlate the levels of overall power, or power in specific frequency ranges (e.g., alpha, delta, beta, gamma, and/or high gamma) with symptom severity and need for treatment with electrical stimulation. In some embodiments, the level of gamma frequency power (such as 30 Hz to 200 Hz) is correlated with symptom severity to determine if a patient is in need of treatment with electrical stimulation. Alternatively or additionally, coherence within certain spectral frequency bands or other features of network connectivity may be correlated with symptom severity and need for treatment with electrical stimulation.

Analyzing the recorded brain electrical activity may comprise the use of an algorithm or classifier. In some embodiments, a machine learning algorithm is used to classify brain activity as indicating whether or not a symptom (e.g., excessive daytime sleepiness or nighttime wakefulness) of a sleep-wake disorder is severe enough that electrical stimulation should be delivered to the brain of the patient. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naïve Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naïve Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

### Administration of a Pharmacological Agent

Embodiments of the methods and systems provided in this disclosure may also include administration of an effective amount of at least one pharmacological agent. By “effective amount” is meant a dosage sufficient to prevent or treat a sleep-wake disorder in a subject as desired. The effective amount will vary somewhat from subject to subject, and may depend upon factors such as the age and physical condition of the subject, severity of the sleep-wake disorder being treated, the duration of the treatment, the nature of any concurrent treatment, the form of the agent, the pharmaceutically acceptable carrier used if any, the route and method of delivery, and analogous factors within the knowledge and expertise of those skilled in the art. Appropriate dosages may be determined in accordance with routine pharmacological procedures known to those skilled in the art, as described in greater detail below.

If a pharmacological approach is employed in the treatment of a sleep-wake disorder, the specific nature and dosing schedule of the agent will vary depending on the particular nature of the disorder to be treated. Representative pharmacological agents that may find use in certain embodiments of the subject invention include, but are not limited to, methylphenidate, dextroamphetamine, solriamfetol, pitolisant, modafinil, armodafinil, sodium oxybate, eszopiclone, zaleplon, zolpidem, zopiclone, and the like.

In certain aspects, the administration of a pharmacological agent involves using a pharmacological delivery device such as, but not limited to, pumps (implantable or external devices), epidural injectors, syringes or other injection apparatus, catheter and/or reservoir operatively associated with a catheter, etc. For example, in certain embodiments a delivery device employed to deliver at least one pharmacological agent to a subject may be a pump, syringe, catheter or reservoir operably associated with a connecting device such as a catheter, tubing, or the like. Containers suitable for delivery of at least one pharmacological agent to a pharmacological agent administration device include instruments of containment that may be used to deliver, place, attach, and/or insert the at least one pharmacological agent into the delivery device for administration of the pharmacological agent to a subject and include, but are not limited to, vials, ampules, tubes, capsules, bottles, syringes and bags. Administration of a pharmacological agent may be performed by a user or by a closed loop system.

### Systems

The present disclosure also provides systems which find use, e.g., in practicing the subject methods. The system may be a closed-loop system configured for performing the methods provided herein. In some embodiments, the closed-loop system may include a stimulation electrode adapted for positioning at the VC, SGC, or OFC region, or other region of the brain of the subject and a detection electrode adapted for positioning at a secondary area of the brain (e.g., VC, SGC, amygdala, OFC, or striato-limbic region, or other region) and for recording an electrical signal from the secondary area before, during, or after an electrical stimulation is applied to the VC, SGC, or OFC region, or other region. The system may also include a computing means and control unit programmed to instruct the stimulation electrode to apply an electrical stimulation to the VC, SGC, or OFC region, or other region in a manner effective to treat the sleep-wake disorder in the subject and/or ameliorate a symptom of the sleep-wake disorder (e.g., excessive daytime sleepiness or lack of energy, excessive nighttime wakefulness, inability to fall asleep or stay asleep long enough, and the like); receive the electrical signals from the secondary area of the brain of the subject via the detection electrode; apply electrical signal metrics to a control algorithm that is tuned to a clinically relevant target (e.g., a range of signal indicative of severity of symptoms and the need for treatment); and automatically delivering electrical stimulation to the brain via the control unit, pulse generator and stimulation electrode in a manner effective to treat the sleep-wake disorder or ameliorate a symptom of the sleep-wake disorder if the electrical signal metrics indicate that the patient is in need of treatment. For example, electrical activity in the gamma frequency range (such as 30 Hz to 200 Hz) or beta frequency range (such as 13 Hz to 30 Hz) from the VC, SGC, amygdala, OFC, or striato-limbic region, or other region may be measured with a detection electrode using this system, wherein the control algorithm receives the electrical activity data from the detection electrode and automates delivery of electrical stimulation via the control unit, a pulse generator and the stimulation electrode to the VC, SGC, or OFC region, or other region when the level of gamma frequency power (such as 30 Hz to 200 Hz) or or beta frequency range (such as 13 Hz to 30 Hz) is in a certain range indicating that a patient has a symptom of a sleep-wake disorder severe enough that the patient is in need of treatment. In some embodiments, one or more programmed stimulation parameters are modulated according to the algorithm's control law based on the recorded electrical activity data, and modulated electrical stimulation is delivered to the VC, SGC, or OFC region, or other region via the control unit, pulse generator and stimulation electrode in a manner effective to ameliorate the symptom of the sleep-wake disorder. The closed loop system may include an on-body pulse generator that is connected to the implanted stimulation electrodes and hence can apply electric stimulation to the brain automatically upon receiving a communication from the control unit.

The processor of the closed-loop system may run programming for assessing the effectiveness of treatment and/or effectiveness of amelioration of a symptom of the sleep-wake disorder and modulate a parameter of the treatment as needed without user intervention. Thus, the closed-loop system may not necessarily include a user interface for a user to instruct the stimulation electrode to apply an electrical stimulation to the VC, SGC, or OFC region, or other region to treat the sleep-wake disorder in the subject. However, in some embodiments, a user interface may be included in the closed-loop system which may be used to confirm the recommendation of the closed loop system or to override it or to change the recommendation.

In certain aspects, a control algorithm for the methods and systems of the present disclosure may include steps of comparing an electrical signal from a region of the brain of a subject to a normal or reference electrical signal, wherein when the electrical signal is significantly different from the normal or reference electrical signal, the control algorithm includes steps of directing a device to apply electrical stimulation to the brain of the subject, followed by measurement of electrical signal from the region of the brain and comparing it to a normal or reference electrical signal, where when the measured signal is significantly different from normal or reference electrical signal, the algorithm includes step of applying another electrical stimulation to the brain.

In some embodiments, the control algorithm utilizes a machine learning algorithm to analyze inputted brain electrical activity data to automate detection of brain activity features that distinguish symptom states of a sleep-wake disorder that are severe enough to need treatment from symptomless states (baseline) or minor symptom states that do not need treatment. The control algorithm then directs a device to apply electrical stimulation to the brain of the subject if the brain activity features indicate a symptom state severe enough that the subject should be treated with electrical stimulation. For example, a machine learning algorithm may be used to correlate the levels of overall power, or power in specific frequency ranges (e.g., alpha, delta, beta, gamma, and/or high gamma) with symptom severity and need for treatment with electrical stimulation. In some embodiments, the level of gamma frequency power (such as 30 Hz to 200 Hz) is correlated with symptom severity to determine if a patient is in need of treatment with electrical stimulation. In some embodiments, electrical stimulation is delivered when the level of an electrical signal that is detected exceeds a set threshold level.

Components of systems for carrying out the presently disclosed methods are further described in the examples below.

### Utility

The methods and systems of the present disclosure find use in a variety of different applications, including the treatment of a sleep-wake disorder. Sleep-wake disorders that may be treated using the methods and systems presented herein include any disease or condition that causes problems with poor quality of sleep, timing of sleep, and/or the amount of sleep of a subject, which may result in impaired daytime functioning. Sleep-wake disorders include, but are not limited to, hypersomnia, excessive daytime sleepiness, insomnia, sleep apnea, parasomnia, narcolepsy, restless leg syndrome, non-rapid eye movement (NREM) sleep arousal disorder, and rapid and eye movement (REM) sleep behavior disorder.

In certain aspects, the methods and systems of the present disclosure find use treating a subject suffering from a moderate to severe form of a sleep-wake disorder, for example the subject may be suffering from moderate or severe hypersomnia or insomnia. In certain aspects, a subject may be suffering from a mild form of a sleep-wake disorder and may be treated according to the methods or using the systems of the present disclosure to treat the mild disorder and prevent progression into a more severe form.

In certain aspects, the methods and systems of the present disclosure may be used to treat hypersomnia, such as caused by idiopathic hypersomnia, narcolepsy, Kleine-Levin syndrome, Prader-Willi syndrome, Norrie disease, Niemann-Pick disease type C, myotonic dystrophy, fibromyalgia, chronic fatigue syndrome, lupus, rheumatoid arthritis, Morvan's syndrome, hypothyroidism, multiple sclerosis, encephalitis, Alzheimer's disease, Parkinson's disease, hydrocephalus, anemia, encephalitis lethargica, paraneoplastic syndrome, head trauma, or depression. Efficacy of the treatment of patients suffering from hypersomnia may be measured in an art accepted manner such as, by a visual-analog scale (VAS), a psychomotor vigilance test (PVT), a Likert scale, a Stanford Sleepiness Scale (SSS), a maintenance of wakefulness test (MWT), an Epworth sleepiness scale (ESS), or a multiple sleep latency test (MSLT).

In certain aspects, the methods and systems of the present disclosure may be used to treat insomnia, which may include sleep onset insomnia, middle-of-the-night awakening, early morning awakening, and/or poor sleep quality (e.g., not reaching stage 3 or delta sleep). Efficacy of the treatment of patients suffering from hypersomnia may be measured in an art accepted manner such as, by a visual-analog scale (VAS), a psychomotor vigilance test (PVT), a Likert scale, a Stanford Sleepiness Scale (SSS), a maintenance of wakefulness test (MWT), an Epworth sleepiness scale (ESS), a multiple sleep latency test (MSLT), or an Athens insomnia scale.

Electrical stimulation methods, including implantation of electrodes, are considerably less invasive than targeted ablation approaches. Furthermore, study results show that in examples of acute stimulation, effects on sleep-wake levels are similarly acute, indicating that the method represents a reversible intervention. This attribute offers the added advantages of (a) precise tuning of therapeutic stimulation to suit the needs of the patient and (b) the incorporation of stimulation into well-controlled clinical trials featuring on/off periods and/or treatment crossover design.

Closed-loop stimulation methods consistently show effectiveness in alleviating symptoms of excessive daytime sleepiness or excessive nighttime wakefulness in affected patients. In addition, stimulation has the potential to be targeted and tuned in a personalized manner that may achieve reliable effective symptom relief compared to other treatment methods.

Cognitive behavioral therapies (CBT) and pharmacologic interventions, for example, with stimulants or sedatives, are among the most established and widely implemented therapeutic approaches for sleep-wake disorders, though often cause undesired side-effects (e.g., treatment with stimulants may cause insomnia; treatment with sedatives may cause daytime sleepiness, dizziness, motor incoordination, cognitive impairment, induce dependency, and worsen sleep quality in the long-term). Notably, stimulation presumably achieves its symptom-relieving effects through distinct mechanisms from those enacted by CBT or pharmacologic interventions. Hence, stimulation represents a potentially complementary therapeutic modality that could be combined with CBT and/or pharmacologic treatment to optimize patient benefit.

Similarly, stimulation methods may be optimized through the incorporation of complementary assessment techniques, including physiological and behavioral measures, performance on functionally relevant cognitive tasks, and patient-directed neurofeedback. Moreover, when integrated in real time, these assessment approaches can refine targeting and/or tuning of stimulation and its therapeutic effectiveness.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-104 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of modulating sleep-wake levels in a subject, the method comprising applying electrical stimulation to a location in a ventral capsule (VC) region, an orbitofrontal cortex (OFC) region, or a subgenual cingulate (SGC) region of the subject using an electrode in a manner effective to modulate sleep-wake levels of the subject.

2. The method of aspect 1, wherein the location in the VC region is in a right VC region, a left VC region, or a combination thereof.

3. The method of aspect 1 or 2, wherein said applying the electrical stimulation comprises applying electrical stimulation at a frequency in a range from 70 Hz to 250 Hz such that wakefulness or energy of the subject is increased, or reaction time of the subject is decreased compared to wakefulness, energy, or reaction time of the subject before said applying the electrical stimulation.

4. The method of aspect 1 or 2, wherein said applying the electrical stimulation comprises applying electrical stimulation at a frequency in a range of 0.5 Hz to 10 Hz such that sleepiness or reaction time of the subject is increased compared to sleepiness or reaction time of the subject before said applying the electrical stimulation.

5. The method of any one of aspects 1-4, wherein said applying the electrical stimulation comprises applying electrical stimulation for 5 seconds to 120 seconds.

6. The method of any one of aspects 1-5, wherein said applying the electrical stimulation comprises applying electrical stimulation intermittently with a rest period between stimulations.

7. The method of aspect 6, wherein the rest period ranges from 10 seconds to 10 minutes.

8. The method of aspect 7, wherein said applying the electrical stimulation comprises applying electrical stimulation intermittently with a stimulation period of 6 seconds and a rest period of 10 seconds, or a stimulation period of 30 seconds and a rest period of 2 minutes.

9. The method of any one of aspects 1-8, further comprising mapping the VC region, the OFC region, or the SGC region of the subject to identify an optimal location for positioning the electrode for delivery of the electrical stimulation to modulate sleep-wake levels of the subject.

10. The method of any one of aspects 1-9, wherein the electrode is placed on a surface of the VC region, the OFC region, or the SGC region.

11. The method of any one of aspects 1-9, wherein the electrode is placed within the VC region, the OFC region, or the SGC region.

12. The system of any one of aspects 1-11, wherein the electrode is a non-brain penetrating surface electrode array or a brain-penetrating electrode array.

13. The method of any one of aspects 1-12, wherein the subject has a sleep-wake disorder, wherein applying the electrical stimulation treats the sleep-wake disorder.

14. The method of aspect 13, wherein the sleep-wake disorder is hypersomnia, excessive daytime sleepiness, insomnia, sleep apnea, parasomnia, narcolepsy, restless leg syndrome, non-rapid eye movement (NREM) sleep arousal disorder, or rapid eye movement (REM) sleep behavior disorder.

15. The method of aspect 14, wherein the hypersomnia is caused by idiopathic hypersomnia, narcolepsy, Kleine-Levin syndrome, Prader-Willi syndrome, Norrie disease, Niemann-Pick disease type C, myotonic dystrophy, fibromyalgia, chronic fatigue syndrome, lupus, rheumatoid arthritis, Morvan's syndrome, hypothyroidism, multiple sclerosis, encephalitis, Alzheimer's disease, Parkinson's disease, hydrocephalus, anemia, encephalitis lethargica, paraneoplastic syndrome, head trauma, or depression.

16. The method of aspect 14, wherein the insomnia is caused by deep brain stimulation therapy to an anterior limb of internal capsule (ALIC) region of the subject.

17. The method of aspect 16, wherein the subject has obsessive-compulsive disorder or major depressive disorder.

18. The method of any one of aspects 1-17, wherein the electrical stimulation is applied unilaterally or bilaterally.

19. The method of any one of aspects 1-18, further comprising assessing effectiveness of the electrical stimulation in said modulating the sleep-wake levels of the subject.

20. The method of aspect 19, wherein said assessing comprises measuring a level of sleepiness or wakefulness of the subject using a visual-analog scale (VAS), a psychomotor vigilance test (PVT), a Likert scale, a Stanford Sleepiness Scale (SSS), a maintenance of wakefulness test (MWT), an Epworth sleepiness scale (ESS), a multiple sleep latency test (MSLT), or an Athens insomnia scale.

21. The method of any one of aspects 1-20, further comprising monitoring sleepiness or wakefulness of the subject using actigraphy or polysomnography.

22. The method of any one of aspects 1-21, further comprising monitoring wakefulness or sleepiness of the subject by recording a brain electrical signal associated with wakefulness or sleepiness of the subject, wherein the brain electrical signal is from a ventral capsule (VC) region, a subgenual cingulate (SGC) region, an amygdala region, an orbitofrontal cortex (OFC) region, or a striato-limbic region of the subject.

23. The method of any one of aspects 1-22, wherein the brain electrical signal associated with wakefulness or sleepiness comprises alpha frequency, beta frequency, gamma frequency, delta frequency, or theta frequency neural oscillations.

24. The method of aspect 23, wherein the gamma frequency neural oscillations are low gamma frequency neural oscillations in a range from 31 Hz to 81 Hz or high gamma frequency neural oscillations in a range from 81 Hz to 150 Hz.

25. The method of aspect 23 or 24, further comprising measuring a relative spectral power level for the gamma frequency neural oscillations, wherein higher relative power of the gamma frequency neural oscillations is associated with lower sleepiness and higher wakefulness compared to lower relative power of the gamma frequency neural oscillations.

26. The method of any one of aspects 22-25, wherein said applying electrical stimulation comprises applying electrical stimulation when the recorded brain electrical signal associated with wakefulness or sleepiness of the subject exceeds a threshold level.

27. The method of any one of aspects 22-26, wherein the subject is monitored continuously or intermittently.

28. The method of any one of aspects 1-27, wherein said applying electrical stimulation comprises applying non-invasive brain stimulation (NIBS) to the VC region, the OFC region, or the SGC region.

29. The method of any one of aspects 1-28, wherein said applying electrical stimulation comprises applying non-invasive brain stimulation (NIBS) to a right VC region, a left VC region, or a combination thereof.

30. A closed-loop method for treating hypersomnia in a subject, the method comprising:
recording a brain electrical signal associated with sleepiness of the subject using a first electrode, wherein the brain electrical signal is from a ventral capsule (VC) region, a subgenual cingulate (SGC) region, an amygdala region, an orbitofrontal cortex (OFC) region, or a striato-limbic region, of the subject; and
applying electrical stimulation to a location in the VC region, the OFC region, or the SGC region of the subject using a second electrode in a manner effective to treat the hypersomnia in the subject when the electrical signal that is detected using the first electrode exceeds a threshold level.

31. The method of aspect 30, wherein the location in the VC region is in a right VC region, a left VC region, or a combination thereof.

32. The method of aspect 30 or 31, wherein said applying the electrical stimulation comprises applying electrical stimulation at a frequency in a range from 70 Hz to 250 Hz such that wakefulness or energy of the subject is increased compared to wakefulness or energy of the subject before said applying the electrical stimulation.

33. The method of any one of aspects 30-32, wherein said applying the electrical stimulation comprises applying electrical stimulation for 5 seconds to 120 seconds.

34. The method of any one of aspects 30-33, further comprising mapping the ventral capsule (VC) region, a subgenual cingulate (SGC) region, an amygdala region, an orbitofrontal cortex (OFC) region, or a striato-limbic region of the subject to identify an optimal location for positioning the first electrode for recording the brain electrical signal associated with sleepiness of the subject.

35. The method of any one of aspects 30-34, further comprising mapping the VC region, the OFC region, or the SGC region of the subject to identify an optimal location for positioning the second electrode for delivery of the electrical stimulation to treat the hypersomnia of the subject.

36. The method of any one of aspects 30-35, wherein the electrode is placed on a surface of the VC region, the OFC region, or the SGC region.

37. The method of any one of aspects 30-35, wherein the electrode is placed within the VC region, the OFC region, or the SGC region.

38. The system of any one of aspects 30-37, wherein the electrode is a non-brain penetrating surface electrode array or a brain-penetrating electrode array.

39. The method of any one of aspects 30-38, further comprising using a control algorithm to automate said applying electrical stimulation when the brain electrical signal associated with sleepiness exceeds the threshold level.

40. The method of aspect 39, wherein the control algorithm comprises a machine learning algorithm for classification of degree of sleepiness based on the brain electrical signal.

41. The method of aspect 40, wherein the machine learning algorithm is a supervised machine learning algorithm.

42. The method of any one of aspects 39-41, wherein the control algorithm further modulates one or more programmed stimulation parameters based on a level of power of the brain electrical signal.

43. The method of any one of aspects 30-42, wherein the brain electrical signal associated with sleepiness comprises alpha frequency, beta frequency, gamma frequency, delta frequency, or theta frequency neural oscillations.

44. The method of aspect 43, wherein the gamma frequency neural oscillations are low gamma frequency neural oscillations in a range from 31 Hz to 81 Hz. or high gamma frequency neural oscillations in a range from 81 Hz to 150 Hz.

45. The method of aspect 43 or 44, further comprising measuring a relative spectral power level for the gamma frequency neural oscillations, wherein higher relative power of the gamma frequency neural oscillations is associated with lower sleepiness and higher wakefulness compared to lower relative power of the gamma frequency neural oscillations.

46. The method of any one of aspects 30-45, wherein the subject is monitored continuously or intermittently.

47. The method of any one of aspects 30-46, wherein the electrical stimulation is applied unilaterally or bilaterally.

48. The method of any one of aspects 30-47, wherein said applying electrical stimulation comprises applying non-invasive brain stimulation (NIBS) to the VC region, the OFC region, or the SGC region.

49. The method of any one of aspects 30-48, wherein said applying electrical stimulation comprises applying non-invasive brain stimulation (NIBS) to a right VC region, a left VC region, or a combination thereof.

50. The method of any one of aspects 30-49, further comprising assessing effectiveness of the electrical stimulation in said treating hypersomnia in the subject.

51. The method of aspect 50, wherein said assessing comprises measuring a level of sleepiness or wakefulness of the subject using a visual-analog scale (VAS), a psychomotor vigilance test (PVT), a Likert scale, a Stanford Sleepiness Scale (SSS), a maintenance of wakefulness test (MWT), an Epworth sleepiness scale (ESS), or a multiple sleep latency test (MSLT).

52. The method of any one of aspects 30-51, further comprising monitoring sleepiness or wakefulness of the subject using actigraphy or polysomnography.

53. A closed-loop method for treating insomnia in a subject, the method comprising:
recording a brain electrical signal associated with wakefulness of the subject using a first electrode, wherein the brain electrical signal is from a ventral capsule (VC) region, a subgenual cingulate (SGC) region, an amygdala region, an orbitofrontal cortex (OFC) region, or a striato-limbic region of the subject; and
applying electrical stimulation to a location in the VC region, the OFC region, or the SGC region of the subject using a second electrode in a manner effective to treat the insomnia in the subject when the electrical signal that is detected using the first electrode exceeds a threshold level.

54. The method of aspect 53, wherein the location in the VC region is in a right VC region, a left VC region, or a combination thereof.

55. The method of aspect 53 or 54, wherein said applying the electrical stimulation comprises applying electrical stimulation at a frequency in a range of 0.5 Hz to 10 Hz such that sleepiness of the subject is increased compared to sleepiness of the subject before said applying the electrical stimulation.

56. The method of any one of aspects 53-55, wherein said applying the electrical stimulation comprises applying electrical stimulation for 5 seconds to 120 seconds.

57. The method of any one of aspects 53-56, further comprising mapping the ventral capsule (VC) region, a subgenual cingulate (SGC) region, an amygdala region, an orbitofrontal cortex (OFC) region, or a striato-limbic region of the subject to identify an optimal location for positioning the first electrode for recording the brain electrical signal associated with wakefulness of the subject.

58. The method of any one of aspects 53-57, further comprising mapping the VC region, the OFC region, or the SGC region of the subject to identify an optimal location for positioning the second electrode for delivery of the electrical stimulation to treat the insomnia in the subject.

59. The method of any one of aspects 53-58, wherein the electrode is placed on a surface of the VC region, the OFC region, or the SGC region.

60. The method of any one of aspects 53-59, wherein the electrode is placed within the VC region, the OFC region, or the SGC region.

61. The system of any one of aspects 53-60, wherein the electrode is a non-brain penetrating surface electrode array or a brain-penetrating electrode array.

62. The method of any one of aspects 53-61, further comprising using a control algorithm to automate said applying electrical stimulation when the brain electrical signal associated with wakefulness exceeds the threshold level.

63. The method of aspect 62, wherein the control algorithm comprises a machine learning algorithm for classification of degree of wakefulness based on the brain electrical signal.

64. The method of aspect 63, wherein the machine learning algorithm is a supervised machine learning algorithm.

65. The method of any one of aspects 63-64, wherein the control algorithm further modulates one or more programmed stimulation parameters based on a level of power of the 66. The method of any one of aspects 53-65, wherein the brain electrical signal associated with wakefulness comprises alpha frequency, beta frequency, gamma frequency, delta frequency, or theta frequency neural oscillations.

67. The method of aspect 66, wherein the gamma frequency neural oscillations are low gamma frequency neural oscillations in a range from 31 Hz to 81 Hz or high gamma frequency neural oscillations in a range from 81 Hz to 150 Hz.

68. The method of aspect 66 or 67, further comprising measuring a relative spectral power level for the gamma frequency neural oscillations or beta frequency neural oscillations, wherein higher relative power of the gamma frequency neural oscillations or beta frequency neural oscillations is associated with lower sleepiness and higher wakefulness compared to lower relative power of the gamma frequency neural oscillations or beta frequency neural oscillations.

69. The method of any one of aspects 53-68, wherein the subject is monitored continuously or intermittently.

70. The method of any one of aspects 53-69, wherein the electrical stimulation is applied unilaterally or bilaterally.

71. The method of any one of aspects 53-70, wherein said applying electrical stimulation comprises applying non-invasive brain stimulation (NIBS) to the VC region, the OFC region, or the SGC region.

72. The method of any one of aspects 53-71, wherein said applying electrical stimulation comprises applying non-invasive brain stimulation (NIBS) to a right VC region, a left VC region, or a combination thereof.

73. The method of any one of aspects 53-72, further comprising assessing effectiveness of the electrical stimulation in treating the insomnia in the subject.

74. The method of aspect 73, wherein said assessing comprises measuring a level of sleepiness or wakefulness of the subject using a visual-analog scale (VAS), a psychomotor vigilance test (PVT), a Likert scale, a Stanford Sleepiness Scale (SSS), a maintenance of wakefulness test (MWT), an Epworth sleepiness scale (ESS), a multiple sleep latency test (MSLT), or an Athens insomnia scale.

75. The method of any one of aspects 53-74, further comprising monitoring sleepiness or wakefulness of the subject using actigraphy or polysomnography.

76. The method of any one of aspects 53-75, wherein the insomnia is caused by deep brain stimulation therapy to an anterior limb of internal capsule (ALIC) region of the subject.

77. The method of aspect 76, wherein the subject has obsessive-compulsive disorder or major depressive disorder.

78. A system for modulating sleep-wake levels in a subject, the system comprising:
a first electrode adapted for recording a brain electrical signal associated with wakefulness or sleepiness of the subject, wherein the brain electrical signal is from a ventral capsule (VC) region, a subgenual cingulate (SGC) region, an amygdala region, an orbitofrontal cortex (OFC) region, or a striato-limbic region of the subject;
a second electrode adapted for applying electrical stimulation to a location in the VC region, the OFC region, or the SGC region of the subject; and
a processor programmed to instruct the second electrode to apply the electrical stimulation to the VC region, the OFC region, or the SGC region of the subject in a manner effective to modulate sleep-wake levels in the subject when the brain electrical signal associated with wakefulness or sleepiness of the subject that is detected using the first electrode exceeds a threshold level.

79. The system of aspect 78, wherein the system further comprises a user interface comprising an input electronically coupled to the processor for instructing the second electrode to apply the electrical stimulation to the location in the VC region, the OFC region, or the SGC region of the subject.

80. The system of aspect 79, wherein the user interface is password protected and is operable by a health care practitioner.

81. The system of any one of aspects 78-80, wherein the processor is further programmed to modulate one or more programmed stimulation parameters according to the algorithm's control law; and apply the modulated electrical stimulation to the VC region, the OFC region, or the SGC region of the subject using the second electrode in a manner effective to treat a sleep-wake disorder.

82. The system of any one of aspects 78-81, wherein the processor is further programmed to apply the electrical stimulation at a frequency in a range from 70 Hz to 250 Hz such that wakefulness or energy of the subject is increased compared to wakefulness or energy of the subject before said applying the electrical stimulation.

83. The system of any one of aspects 78-82, wherein the processor is further programmed to apply the electrical stimulation at a frequency in a range of 0.5 Hz to 10 Hz such that sleepiness of the subject is increased compared to sleepiness of the subject before said applying the electrical stimulation.

84. The system of any one of aspects 78-83, wherein the processor is further programmed to apply the electrical stimulation for 5 seconds to 120 seconds.

85. The system of any one of aspects 78-84, wherein the processor is further programmed to apply the electrical stimulation intermittently with a rest period between stimulations.

86. The system of aspect 85, wherein the rest period ranges from 10 seconds to 10 minutes.

87. The system of aspect 86, wherein the processor is further programmed to apply the electrical stimulation intermittently with a stimulation period of 6 seconds and a rest period of 10 seconds, or a stimulation period of 30 seconds and a rest period of 2 minutes.

88. The system of any one of aspects 78-87, wherein the processor is further programmed to set a maximum number of electrical stimulations per day.

89. The system of any one of aspects 78-88, wherein the processor is further programmed to set a total amount of time of electrical stimulation per day.

90. The system of any one of aspects 78-89, wherein the first electrode is adapted for positioning on a surface of the VC region, the SGC region, the amygdala region, or the OFC region of the subject or within the VC region, the SGC region, the amygdala region, the OFC region, or the striato-limbic region of the subject.

91. The system of any one of aspects 78-90, wherein the second electrode is adapted for positioning on a surface of the VC region, the OFC region, or the SGC region or within the VC region, the OFC region, or the SGC region.

92. The system of any one of aspects 78-91, wherein the first electrode or the second electrode is a non-brain penetrating surface electrode array or a brain-penetrating electrode array.

93. The system of any one of aspects 78-92, wherein the first electrode or the second electrode is a non-brain penetrating electrode.

94. The system of any one of aspects 78-92, wherein the first electrode or the second electrode is a brain penetrating electrode.

95. The system of any one of aspects 78-94, wherein the second electrode applies non-invasive brain stimulation (NIBS) to the VC region, the OFC region, or the SGC region.

96. The system of any one of aspects 78-95, wherein the second electrode applies non-invasive brain stimulation (NIBS) to the right VC region, the left VC region, or a combination thereof.

97. The system of any one of aspects 78-96, wherein the sleep-wake disorder is restless leg syndrome, non-rapid eye movement (NREM) sleep arousal disorder, rapid eye movement (REM) sleep behavior disorder.

98. The system of any one of aspects 78-97, wherein the insomnia is caused by deep brain stimulation therapy to an anterior limb of internal capsule (ALIC) region of the subject.

99. The system of aspect 98, wherein the subject has obsessive-compulsive disorder or major depressive disorder.

100. The system of any one of aspects 78-99, wherein the processor is further programmed to set a maximum number of electrical stimulations per day.

101. The system of any one of aspects 78-100, wherein the processor is further programmed to set a total amount of time of electrical stimulation per day.

102. A closed-loop method for treating obsessive-compulsive disorder or major depressive disorder in a subject, the method comprising:
applying electrical stimulation to an anterior limb of internal capsule (ALIC) region of the subject using a first electrode in a manner effective to treat the obsessive-compulsive disorder or major depressive disorder in the subject;
recording a beta frequency brain electrical signal associated with wakefulness of the subject using a second electrode, wherein the beta frequency brain electrical signal is from a striato-limbic region of the subject; and
decreasing amplitude of electrical stimulation during nighttime hours when spectral power of the beta frequency brain electrical signal exceeds a threshold level.

103. The method of aspect 102, wherein the beta frequency brain electrical signal is in a range from 25 Hz to 30 Hz.

104. The method of aspect 102 or 103, further comprising applying electrical stimulation in a range of 0.5 Hz to 10 Hz during nighttime hours when the spectral power of the beta frequency brain electrical signal exceeds the threshold level.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for. Those of skill in the art will readily recognize a variety of noncritical parameters that could be changed or modified to yield essentially similar results.

### Example 1: Intracranial Electrical Stimulation of Corticolimbic Sites Modulates Arousal in Humans

### Introduction

In this study, we tested the hypothesis that regional activation of corticolimbic network sites differentially modulate arousal level, by measuring behavioral stimulation-responses in human participants. If arousal can be differentially modulated through stimulation of corticolimbic circuitry, this may support a circuit mechanism for the adaptive fluctuations of arousal that occur in healthy individuals to emotional stimuli or the disruptions in arousal that occur in individuals with mood disorders.

Indeed, the current conception of arousal networks has extended beyond classical structures (e.g., brainstem, hypothalamus, and basal forebrain) to include distributed pathways of the ascending arousal system, including within limbic structures⁸⁻¹². Dating back to the 1950s, regional limbic activation, such as with the direct stimulation of the amygdala¹³ or mesial temporal lobe seizures¹⁴, was identified to elicit a diffuse cortical arousal pattern of low-amplitude fast activity, reminiscent of the activation of the brainstem reticular system, albeit with complex clinical features. The amygdala was subsequently implicated in the cortical synchronization of low-gamma activity, enabled by its widespread reciprocal connections with the cortex¹⁵. More recently, electrical and optogenetic neuromodulation of subcortical regions that have traditionally been implicated in reward or stress response, including the ventral tegmental area (VTA)¹⁶⁻¹⁹ or bed nucleus of stria terminalis (BNST) 20, have been shown to promote sleep-wake transitions. In addition to circuit level behavioral studies, decades of psychometric research have led to the dissection of emotion into two dimensions of valence and arousal²¹⁻²⁴ and have furthermore supported the coregulation of mood and arousal networks. Yet, the extent to which subjective experience of arousal can be modulated at the level of mood circuits remains unclear.

Human studies may offer increased sensitivity for measuring fluctuations in arousal with direct neurostimulation through quantifying subtle changes in experience or function, rather than relying on isolated electrophysiologic changes or gross behavioral-state changes, e.g., the transition from sleep to wake. In fact, modulation of arousal in humans has been investigated using deep brain stimulation (DBS) techniques. However, these case-series were largely performed in cohorts with impaired consciousness, such as in patients with severe traumatic brain injury or stroke, and thus outcome measures were restricted to gross behavioral observations²⁵⁻²⁷. In addition, anatomical targets to-date have been limited to structures long identified as playing a role in sleep/wake regulation, including the pedunculopontine nucleus (PPN), medial thalamus, basal forebrain, and hypothalamus, with variable success. Other studies in patients with advanced Parkinson's disease (PD) and severe sleep-wake disturbances demonstrated that low frequency stimulation of the PPN decreases daytime sleepiness^{28,29}, suggesting that there is an opportunity for modulating arousal in patients with less severe neurologic disease. Stimulation of downstream targets in subjects who are cognitively intact and can report experiential changes may allow for a more sensitive identification of arousal responses elicited by direct brain stimulation.

In this study, we hypothesized that arousal level can be modulated by regional activation of the corticolimbic network, providing supporting, mechanistic evidence that the corticolimbic network may provide a parallel pathway for augmenting arousal. We tested this hypothesis by leveraging an ongoing clinical trial^{30.31} focused on assessing the antidepressant effects of intracranial stimulation. By assessing subjective experience and clinical behavior, we performed mapping of arousal responses to acute corticolimbic stimulation. Our findings reveal a set of corticolimbic targets, whereby stimulation differentially modulates arousal and electrophysiologic biomarkers in the gamma band correlate with changes in arousal. These results provide initial mechanistic support of the distributed circuitry of the arousal networks across corticolimbic sites, which may explain the significant comorbidity between neuropsychiatric and sleep conditions^{6,7}, as well as adaptive fluctuations in arousal to emotional stimuli.

### Methods

### Participant Cohort

The study design leveraged an ongoing in-hospital clinical trial (NCT04004169) involving the implantation of intracranial electrodes in participants with treatment-resistant depression³⁰. Participants included in the study were required to have treatment-resistant depression without suicidality and prior successful but transient response to electroconvulsive therapy (ECT). During the hospitalization, no sleep-wake medications, including stimulants or sedatives, were administered during or within 10 hours prior to the testing period. Two patients (a 31-year-old Caucasian man and a 29-year old Caucasian woman) met inclusion criteria and were implanted with multi-site, intracranial depth electrodes and underwent a 10 d period of intensive stimulus response mapping in the UCSF Epilepsy Monitoring Unit30. Neither participants were previously diagnosed with a sleep-wake disorder; however, Patient 2 had required stimulants as needed for symptoms of daytime sleepiness and zolpidem as needed for insomnia. In addition, Patient 2 had been maintained on nightly trazadone for symptoms of both mood and insomnia.

### Electrode Placement, Reconstruction, and Tractography

Sixteen-contact depth electrodes (PMT® sEEG Depthalon electrodes, 3.5 mm pitch, center to center) were implanted in the two patients enrolled in the inpatient study. Electrodes were implanted using standard neurosurgical techniques with trajectories targeting the bilateral amygdala (Amy), hippocampus (Hipp), subgenual cingulate (SGC), orbital frontal cortex (OFC), and ventral capsule (VC) with the most distal contacts. Due to differences in electrode trajectories based on individual neuroanatomy, Patient 2 also had left nucleus accumbens (NAC) represented. All subjects underwent presurgical 3 Tesla brain magnetic resonance imaging (MRI), including 1 mm isotropic T1- and T2-weighted imaging, and post-operative computed tomography (CT) scan for electrode localization. Electrode locations were determined by co-registering the presurgical T1-weighted MRI sequence with the post-operative CT. Anatomical locations of each electrode were determined by neuroradiologist review (L.P.S.). Fiber tracts were identified using the Lead DBS toolbox³² in MATLAB (Mathworks, Inc., Natick, MA). Pre-operative MRI and post-operative CT scans were co-registered and normalized to a standardized Montreal Neurological Institute template (MNI152, ICBM 2009b NLIN asymmetric)³³. Putative fiber tracts were derived from a normative group connectome based on 32 subjects from the Human Connectome Project (HCP) (as detailed in Horn et al, 2017³⁴) based on a simple spherical estimate of the volume of tissue activated by a 5 mA unipolar stimulation at the mean location between the individual electrode pair used for bipolar stimulation during the study (according to the stimulation model detailed by Dembek et al., 2017³⁵). For each participant and region, the electrode pair modeled represents the pair that was tested most frequently for that region.

### Behavioral Measures and Responses

Three behavioral ratings were used to capture the degree of arousal of the patient. First, a visual-analog scale (VAS) was completed by the patient to rate the level of energy from 0 (i.e., “not at all energetic”) to 100 (i.e., “very energetic”). Second, the Stanford Sleepiness Scale (SSS) 36, a validated, 7-point self-rated scale of sleepiness, was completed by the patient to determine the degree of sleepiness. Scores of SSS ranged from 1 (“Feeling active, vital, alert or wide awake) to 7 (“No longer fighting sleep, sleep onset soon, having dream-like thoughts”). Third, arousal metrics were scored by a bedside observer using a 5-point Likert scale ranging from −2 (i.e., episodes of eye closure, decreased responsiveness to the environment, slowed speech rate) to 2 (i.e. high level of engagement with the environment, completion of tasks, relatively rapid speech rate). Both participants were blinded to all simulation parameters for all survey assessments. In addition, arousal measures were obtained in a double-blinded fashion. Specifically, the bedside observer sat out of view from the streaming EEG.

At any time, patients were allowed to spontaneously report any experiences or symptoms. Verbal responses were not cued. Spontaneous commentary focused on depression were previously reported in a prior paper³⁰; the remarks included in this manuscript are specific to arousal.

### Stimulation Paradigm and Survey Assessments During Simulation

We note that the stimulation methods employed in this study were originally designed for the treatment of depression; however, through these methods, it was possible to extract additional behavioral scores to evaluate sleep-wake responses to stimulation. As such, the regions that were repeatedly assessed and included in this study for each subject were found to have some degree of anti-depressant effects, and the frequency of stimulation at each region was a function of assessing antidepressant treatment potential for therapy. While this context led to a variation in the amount of data available for each stimulation site, this study offered the unique opportunity to elucidate subjective experience of sleep-wake effects in humans (without epilepsy and without the confounds of anti-seizure medication side effects), which is extremely rare.

Bipolar stimulation of adjacent or nearby electrodes and monitoring of real-time electrophysiologic data were performed using the Nihon Kohden System (Tokyo, Japan). Stimulation was performed on post-operative days 2 through 10 of the inpatient hospitalization for the two patients involved in the inpatient study. Bipolar stimulation was delivered with charge-balanced, biphasic, constant-current trains at a specified frequency, pulse width, and current. Intracranial EEG recordings were collected at 2-10 kHz sampling rates using the Nihon Kohden System and a secondary data stream.

Prior to experimental stimulation, a safety survey was first performed using short duration 1-3 s bursts of stimulation at all anatomically verified electrodes within candidate brain regions to assess for epileptiform discharges. Safety surveys were performed at 1 and 100 Hz, 100 us pulse width, and increasing currents (1-6 mA). For each patient, brain regions and stimulation parameters that elicited epileptiform discharges were avoided in all subsequent experimental stimulation paradigms with longer-duration stimulation³⁷ (see Supplementary Methods).

During stimulation mapping sessions, participants were informed that stimulation would be performed at different brain regions, but blinded to the location and parameters of the stimulation and the time when stimulation started and ended. Stimulation parameters varied by region and current (1-6 mA). All regions were tested at 100 Hz frequency; an additional frequency of 1 Hz was tested for the OFC contact, given a prior anti-depressant effect at low frequencies³⁰. Sham (0 mA) stimulations were randomly included in a subset of the stimulation blocks to serve as an internal control. Baseline periods (i.e. no stimulation) were also interspersed throughout the stimulation testing period, as both a control condition and a source of resting-state data for the biomarker analysis (see below). Finally, washout periods of at least 2 minutes were included into the stimulation schedule to provide resting time for the participants to minimize carryover effects.

Participant surveys assessments were completed at the end of stimulation and baseline periods. To minimize survey fatigue, the frequency of survey assessments was reduced when consecutive stimulations were performed in the same brain region, for instance, with repeated intermittent stimulation paradigms and sequential escalations of stimulation current. To characterize responses of these stimulation paradigms, a single summary score was obtained, as detailed below. Bedside observer arousal scores were performed after each stimulation, including when participant survey responses were not administered.

### Stimulation Response Analysis and Statistics

Behavioral scores and survey responses were aggregated and visualized across regions of stimulation using MATLAB v2019a. As a subset of baseline recordings did not have bedside observer subscores, missing-data was predicted from a regression model using subjective energy and sleepiness measures as predictors for bedside arousal measures. Leave one-out-cross validation was performed to estimate the quality of the imputation (Supplementary Materials).

Stimulation responses were then collected across a range of stimulation paradigms during the 10 d inpatient monitoring. Stimulation paradigms included single trial assessments (e.g., 90 s continuous stimulation), sequential stimulation assessments (e.g. 90 s continuous stimulation at 1 mA, 3 mA, then 6 mA), and intermittent stimulation assessments (e.g. 10 s stimulation/60 s off×50 minutes).

For single trial assessments, stimulation responses were associated with the specifics of the stimulation, which varied by region and stimulation parameters, including current (0.5-6 mA), duration (30 s-3 min), and frequency (1 Hz or 100 Hz). During sequential stimulations, multiple current levels were tested sequentially while holding all other stimulation parameters constant, including electrode contacts, frequency, and duration of stimulation. To account for sequential stimulation results, the survey responses associated with the highest current level were included as a single point to summarize the series of tests. During intermittent stimulation paradigms, short duration stimulations followed by an off period were administered over a longer duration (e.g. 30-60 minutes). To account for intermittent stimulation results, the survey response with the highest deviation from baseline was included as a single point to summarize the overall response.

Principal component analysis (PCA) was performed across the behavioral outcome scores to provide a summary metric of arousal based on multiple subscores. To assess the significance of the behavioral responses, a two-sample Wilcoxon rank sum test was performed on the stimulation-response, summary arousal metrics against a null hypothesis of equivalence of baseline and sham testing. Statistical testing was thresholded for multiple comparison testing with false discovery rate (FDR) of 5%.

### Biomarker Preprocessing and Statistical Analysis

Resting-state electrophysiology and behavioral scores were collected throughout each testing day to capture baseline measures for biomarker analyses. Baseline measures were performed before stimulation testing or at least 10 minutes after the most recent stimulation to minimize the impact of stimulation on the biomarker recording of arousal. Survey assessments were obtained at the end of the baseline recording sessions. The intracranial electrophysiology data obtained over the 5 minutes prior to the initiation of the survey were used in biomarker development.

Offline analysis of electrophysiology data was performed in MATLAB v2019a and Python v3.10. Standard preprocessing of the data was performed, including common average referencing to the mean of all channels, down-sampling to 512 Hz, application of a notch filter to remove artifact from noise and harmonics (60 Hz, 120 Hz, 180 Hz, and 240 Hz), visual identification and rejection of periods with artifact across all channels, rejection of channels with high degree of noise based on a kurtosis threshold, and band-pass filtering of 1-150 Hz.

Spectral power was obtained for individual channels using 25 scales with logarithmically increasing center frequencies between 1-150 Hz and by performing wavelet analysis with Morlet wavelets. Only channels obtained from contacts anatomically verified to be within regions of interest were analyzed (i.e., 63 and 57 total channels across the five anatomical regions for Patients 1 and 2, respectively). Spectral power was averaged across scales to arrive at average estimates of power within canonical frequency bands: delta (1-4 Hz), theta (4-9 Hz), alpha (9-13 Hz), beta (13-31 Hz), low gamma (31-81 Hz), and high gamma (81-150 Hz). Relative power was subsequently obtained by dividing the power in each canonical frequency band by the total power across all six frequency bands. The relative spectral power was averaged across the five-minute biomarker recording period and across channels within anatomical regions of each hemisphere (e.g. SGC, OFC, Hipp, Amy, and VC) for regional statistical testing. To assess the correlation between SSS and spectral biomarkers, Pearson's correlations were assessed across candidate regions and frequency bands. P-values were adjusted for multiple comparison testing with a FDR of 5% across all regions, and the p-values reported reflect adjusted values. SSS was selected as the behavioral outcome to enable a consistent metric for comparison across patients. Electrophysiologic biomarkers that were common across both patients were used as features of investigation in the Electrophysiology Response analysis described in the following paragraph. To further evaluate the biomarkers, the SSS ratings were divided into equal sized high and low sleepiness groups, i.e., a median split. Given that the patients substantially varied in their overall level of sleepiness, the threshold for high and low sleepiness group was patient-specific (Patient 1, high sleepiness state of ≥5 SSS; Patient 2, high sleepiness state of ≥4 SSS). Logistic regression was performed to classify high and low sleepiness levels per patient based on six randomly selected features. Features with the highest predictive values were determined by mean F-values, following 1,000 iterations of feature selection and logistic regression across different train-test splits. The top six features were recalculated in each iteration based on those with the highest F-score in a particular train set. Receiver operating characteristic (ROC) curves were constructed for the strongest logistic regression models to quantify the area under the ROC curve (AUC). Top F-score features were identified by calculating the percentage of the cross-validated models in which each feature was selected.

### Electrophysiologic Biomarker Response to Regional Stimulation

To identify how local spectral power was affected by stimulation, spectrograms were constructed across candidate channels for each stimulation condition with 1) stimulation durations of ≥30 s and 2) pre- and post-stimulation durations of ≥20 s. Stimulation response was investigated on a narrow set of electrographic biomarkers that were identified to be significant across both patients in the Biomarker analysis. For processing stimulation response electrophysiology, spectral power was obtained by performing wavelet transform with the same center frequencies as above and averaged across electrodes representing individual regions. Averaged spectral power for pre-/post-stimulation were obtained using a buffer of 9 s from stimulation, which was visually determined to avoid direct stimulation artifact. Power was z-scored to the pre-stimulation period and subsequently smoothed using a 3 s window size. Post-stimulation z-scored power was compared to pre-stimulation z-scored power, and the difference in pre- and post-stimulation spectral power were assessed by a two-sided, paired T-test, computed separately for each selected feature. Post-hoc multiple comparison thresholding with FDR of 5% was performed across the features investigated.

### Epileptiform Discharges

Stimulation of cortical structures even in patients without a history of epilepsy carries a risk of eliciting epileptiform discharges and seizure activity. In order to safely perform stimulation, all candidate bipolar contacts across all patients were stimulated at increasing currents (1, 3, and 6 mA) for 1 and 3 s in duration, prior to longer duration stimulation. Other stimulation parameters, including frequency (1 and/or 100 Hz depending on site) and pulse width (100 μs), remained unchanged.

### Leave-On-Out Cross Validation (LOOCV) for Imputation of Missing Data

Due to missing observational arousal data in a subset of baseline measures, a regression model was performed with subjective energy and SSS as predictors (arousal=round(β₀+β₁*VAS_{energy}+β₂*SSS)), aggregated subscores across both patients. Following LOOCV, the correlation coefficient between the predicted values (arising from the test set) and true values was 0.219 (p<0.006). The model was subsequently used to impute missing data.

### Results

### Intracranial Mapping to Modulate Arousal

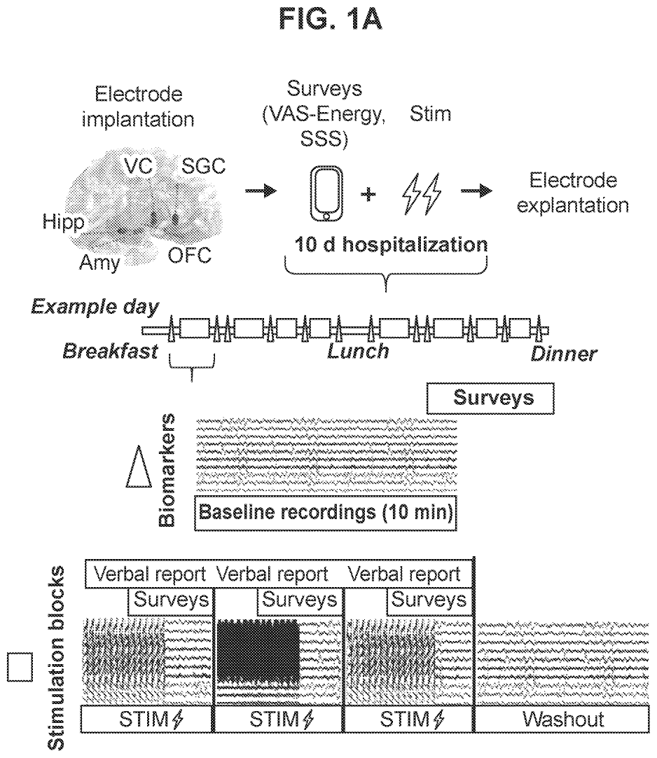

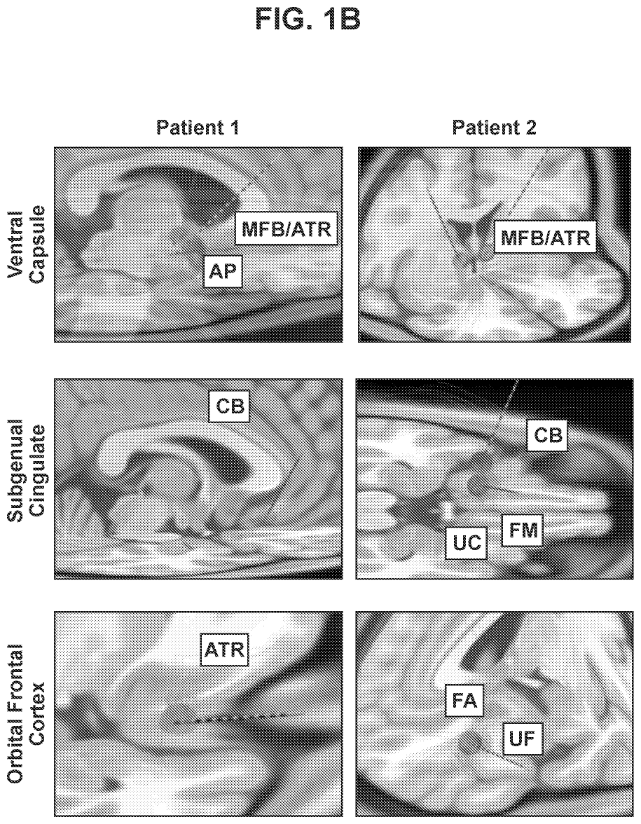

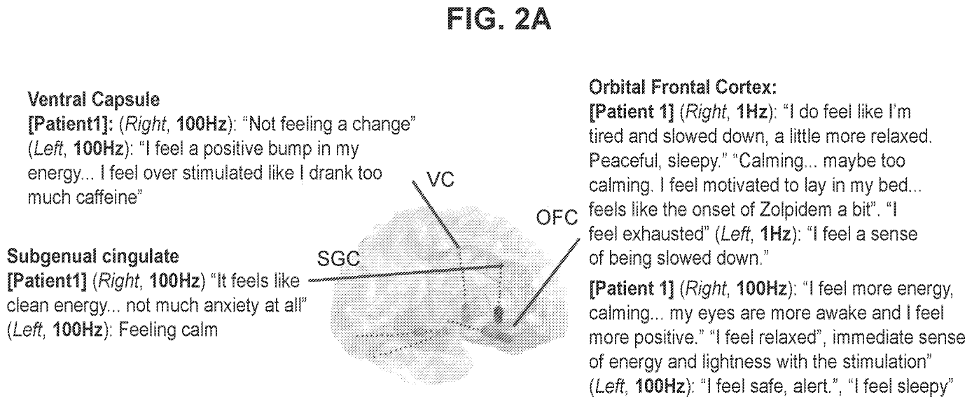

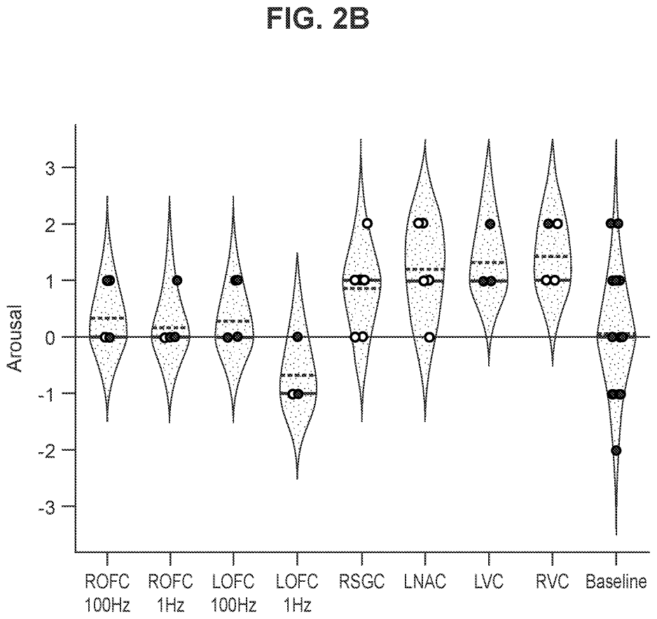

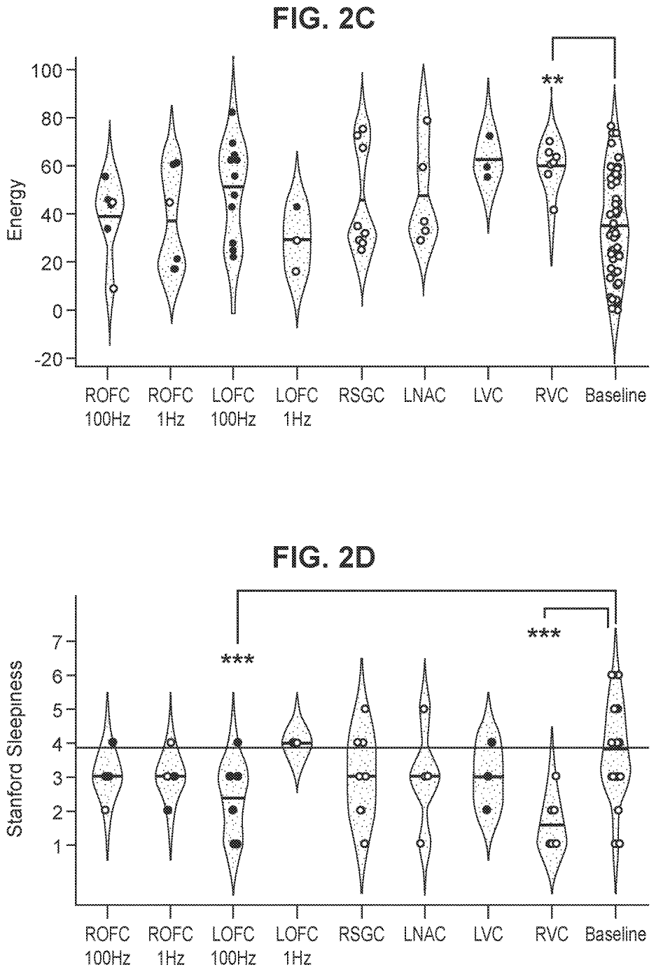

Two patients underwent bilateral symmetric implantation of electrodes targeting candidate corticolimbic structures, followed by an intensive 10 d inpatient monitoring period, during which stimulation-response testing was performed (FIG. 1A). For each participant and region, the predicted volume of tissue activated by stimulation at the select electrode contact pair was used as a seed for fiber-tracking based on the normative connectome. We hypothesized that the resulting tracts reflect those most likely to be recruited by stimulation at the modeled electrode contact pairs. For the VC contacts, fibers belonging to the medial forebrain bundle (Patients 1,2; FIG. 1B, top), anterior thalamic radiations (Patients 1,2), and ansa peduncularis (Patients 1) were identified. For the SGC contacts associated fiber tracts included the cingulum bundle (Patients 1,2; FIG. 1B, middle), forceps minor (Patient 2), and uncinate fasciculus (Patient 2). Finally, for the OFC contacts, associated fiber tracts included the ventral component of the anterior thalamic radiations (Patients 1; FIG. 1B, bottom), uncinate fasciculus (Patients 2), and a branch of the frontal asalant tract (Patient 2). Voluntary spontaneous remarks were made by Patient 1 during stimulation across the VC, amygdala, SGC, and OFC. While verbal responses were variable, notable example comments are presented in FIG. 2A. Patient 2 did not provide spontaneous remarks during stimulation, although they participated in frequent survey assessments. Quantitative outcome assessments (i.e. VAS energy, clinician-rated Likert arousal scale, and SSS) results from Patient 2 revealed increased energy (p=0.002) and decreased sleepiness with right VC stimulation (p<0.001), as well as decreased sleepiness with left OFC 100 Hz stimulation (p<0.001; FIGS. 2A-2C).

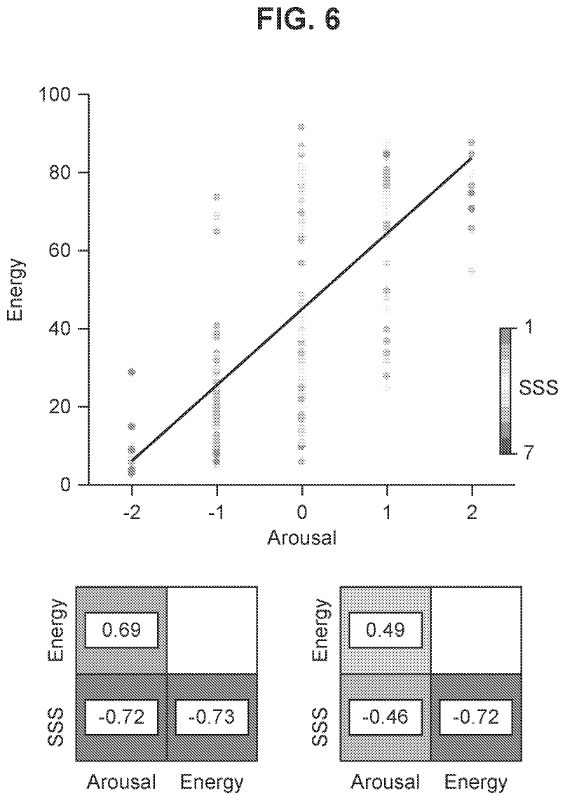

To best capture the regional stimulation response across all subscores, the quantitative behavioral assessments (i.e., arousal, SSS, and energy) were combined into a summary metric for each individual, corresponding to the first principal component (PC1) following dimensionality reduction with PCA. The contributions of each behavioral assessment to PC1 are provided in Table 1 and the dimension captured 81.1% and 70.8% of the variance in the data for Patient 1 and 2, respectively. The individual behavioral assessment inputs into the PCA were moderately correlated for both patients (FIG. 6).

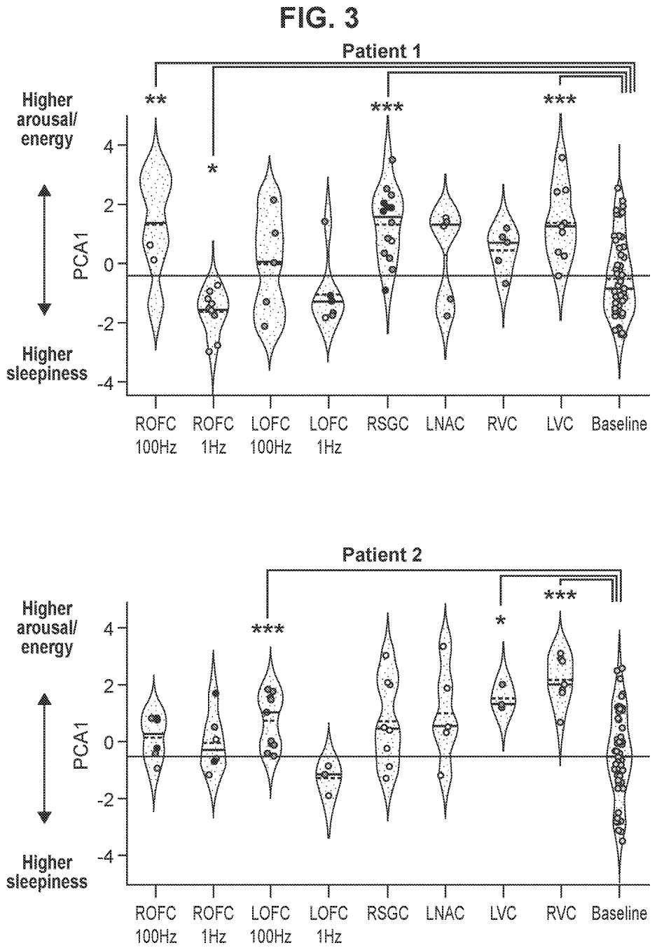

In both patients, right and/or left VC stimulation led to increased arousal/energy responses (FIG. 3; Patient 1, p<0.001; Patient 2, left VC p=0.019 and right VC, p<0.001), as compared to the control. Statistically significant increases in arousal/energy were also observable in the right OFC 100 Hz (Patient 1, p=0.001), left OFC 100 Hz (Patient 2, p=0.008), and right SGC (Patient 1, p<0.001). In addition, Patient 1 exhibited significant decreases in arousal with right OFC 1 Hz stimulation (p=0.011), an opposing effect compared to 100 Hz stimulation within the OFC. Contralateral regions of stimulation, including left OFC 100 Hz and left OFC 1 Hz for Patient 1 and right OFC 100 Hz for Patient 2 were not statistically significant. Only regions that underwent three or more acute stimulation assessments were included in the stimulation analysis.

### Biomarkers of Sleepiness

To characterize electrophysiologic correlates of sleepiness/arousal within the sampled regions, we then performed a biomarker analysis. Relative spectral power from resting-state recordings was correlated with patient-reported sleepiness levels (FIG. 4A; Patient 1, N=59; Patient 2, N=64). The top 20 regions and frequency bands whose spectral power levels are associated with high and low sleepiness for both patients are illustrated in FIGS. 4B, 4C with highest positive and negative correlations depicted in FIGS. 4E, 4F. Across target regions, higher relative power in the low and high gamma frequency bands were associated with low sleepiness or high arousal/wakefulness (i.e. negative correlations with SSS), whereas lower frequency activity in the delta, theta, and alpha frequency bands was more commonly associated with increased sleepiness (i.e. positive correlations with SSS). The top 20 highest correlations by magnitude, averaged across both patients, are depicted in FIG. 4D; the regions and features that are statistically significant across both patients are examined in the subsequent local electrophysiologic response analysis.

A logistic regression classifier was constructed to further characterize the ability of the spectral power features recorded from target regions to differentiate the dichotomous high or low sleepiness level for each patient. Utilizing the six top features most commonly selected across 1000 iterations of cross-validated feature selection, the AUC achieved was 0.78 for Patient 1 and 0.92 for Patient 2 (FIGS. 4G, 4H). The top six features determined for the logistic regression classifier for Patient 1 are the delta, alpha, and beta bands within the right hippocampus, high gamma band in the right OFC, alpha band within the left VC, and the high gamma band within the left OFC (FIG. 4G). The top six features for Patient 2 are the high gamma bands of the left OFC and right hippocampus, as well as the high and low gamma bands of the left amygdala and left hippocampus (FIG. 4H).

### Local Electrophysiology Effects of Stimulation Mapping

To validate the association between the identified biomarker features and arousal level, we next tested whether stimulation paradigms that modulate arousal level also modulated these features. Using the biomarker features that were identified to be statistically significant across both patients (FIG. 4D, denoted by line and asterisks), we assessed the biomarker responses with respect to the most robust, wake-promoting stimulation site, i.e. right or left VC, and compared the response to a sleep-promoting site, i.e. 1 Hz OFC stimulation (FIG. 5). Example spectrograms are constructed for select regions in response to 100 Hz VC (FIG. 5A) and 1 Hz OFC stimulation (FIG. 5C).

Across all patients, the post-stimulation spectral power was compared to the pre-stimulation power within relevant spectral bands (FIGS. 5B, 5D). The group analysis of VC stimulation across all patients (N=43 stimulations) revealed increased spectral power in the high gamma power band within the left VC (p=0.013), left SGC (p=0.026), left amygdala (p=0.027), right OFC (p=0.009), and right VC (p=0.025), as well as increased spectral power in the low gamma power band within the right VC (p=0.031). Changes in spectral power within the gamma band of left VC and right amygdala were not statistically significant. In contrast to the effects of VC stimulation, 1 Hz OFC stimulation (N=29 stimulations) did not elicit a change in the spectral power of any of the relevant sensing locations/frequency bands. Importantly, the increase in spectral power with VC stimulation in these regions/frequency bands are consistent with what would be predicted by the biomarker results, where increased power indicates increased wakefulness and decreased sleepiness.

### Discussion

In this study, we present evidence that acute intracranial stimulation of corticolimbic sites modulates arousal, based on both subjective experience and behavior. Three novel targets where stimulation led to arousal effects were identified, including OFC, SGC, and most robustly, VC. Low frequency (1 Hz) stimulation of the OFC led to increased sleepiness, while high frequency (100 Hz) stimulation of the SGC, OFC, and VC led to increased arousal. Gamma frequency activity in a variety of subregions was demonstrated to be associated with decreased sleepiness/increased wakefulness. The finding that a subset of these corticolimbic regions, which are broadly implicated in mood, motivation, inhibition, reward, and fear and currently investigated as targets for treatment-resistant depression^{31,38-40}, modulates arousal provides initial evidence for shared circuitry between arousal and mood regulation in humans—the likely basis for the strong clinical and behavioral association of arousal and mood⁵⁻⁷. Additionally, the identification of novel targets highlights the feasibility of using neurostimulation in corticolimbic structures to modulate arousal, potentially opening the door to novel neurostimulation strategies for disabling sleep-wake disorders.

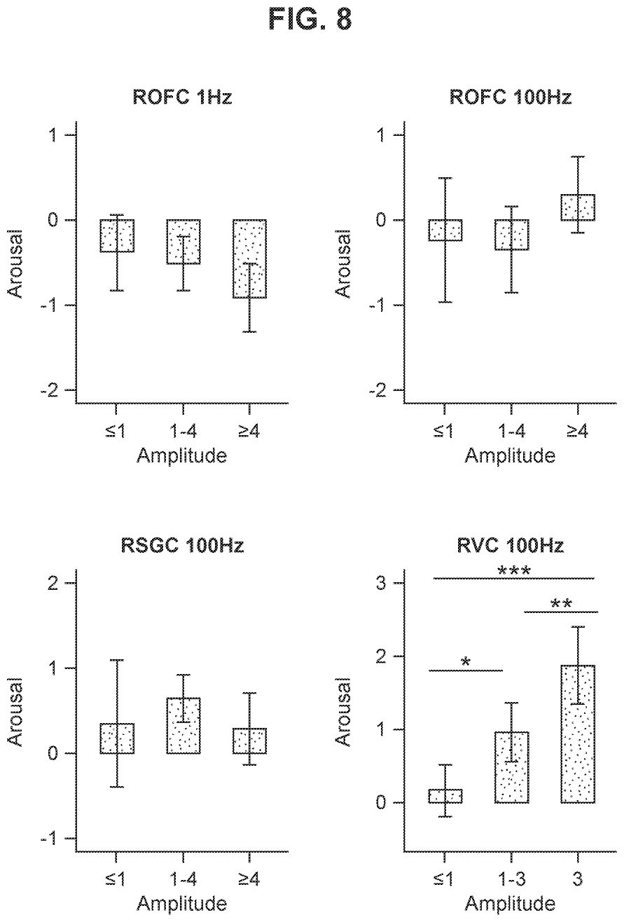

We observed that stimulation of the bilateral VC yielded the most significant and robust increases in quantitative arousal levels. Our findings expand upon and corroborate findings from an initial patient previously published by our group, suggesting a dose response of VC stimulation with clinician-scored arousal metrics³⁰. Anecdotal experience with the initial patient, who subsequently underwent chronic responsive neurostimulation within the right VC³¹ and reported symptoms of insomnia and disrupted sleep with right VC stimulation during evening hours, further reinforce this finding. These symptoms resolved when overnight VC stimulation was prevented. Indeed, the observations during the stimulation-mapping of this initial patient inspired methodologic changes, such as capturing subjective SSS measures, to the subsequent clinical trial participants, as presented in this study. Supplementary analysis with the initial patient is provided in FIGS. 2, 8. In addition, our findings are consistent with other group's qualitative observations of increased engagement in conversation, energy, and awareness with therapeutic VC stimulation for TRD^{41,42} and expand on such reports by providing quantitative measures of arousal levels.

While VC has been a target for DBS applications for depression^{31.39.43,44}, OCD45, and other sleep-wake disorders, the physiological effects of VC stimulation remain poorly understood. VC stimulation is thought to activate traversing white matter tracts and their connected structures. Here, VC stimulation was identified to increase in gamma activity in multiple brain regions (FIG. 6), including the bilateral VCs, amygdala, SGC, and OFC, suggesting a distributed and highly connected network associated with the VC subregion. Prominent tracts identified in models of depression and OCD include the anterior thalamic radiations connecting the anterior and medial nuclei of the thalamus to the frontal lobe^{46,47}, and the ventral tegmental area projection pathway (VTApp) connecting the VTA to the nucleus accumbens (NAc) and key frontal nodes including the subgenual cingulate (in some studies part of this pathway has been referred to as the superolateral branch of the medial forebrain bundle⁴⁷⁻⁵⁰, though the existence of this structure as a discrete anatomic connection has recently been called into doubt⁵¹). Indeed, in our tractography results based on normative connectome data (FIG. 1B), models of tissue activated by stimulation at effective VC contacts were associated with fibers corresponding to both the anterior thalamic radiations and VTApp/MFB. In addition, structures near the VC, such as the bed nucleus of stria terminalis (BNST) and NAc, may be directly affected by the sphere of electrical stimulation.

In fact, the aforementioned corticolimbic structures associated with VC stimulation effects have recently been implicated in sleep-wake control by means of the mesolimbic dopamine pathways¹⁶. Modulation of dopaminergic VTA neurons have been demonstrated to sustain wakefulness, even during periods of high sleep drive^{17,18,52}. Arousal effects of VTA stimulation have been traced to projections within the central amygdala, dorsolateral striatum, and most robustly within NAc, in which optogenetic and chemogenetic modulation of D1-expressing neurons promotes wakefulness in animal models^{18,19}. D1 neurons within the NAc have additionally been observed to project to midbrain and lateral hypothalamus structures¹⁹, thereby converging on classical arousal pathways, while D2 receptor-expressing GABAergic neurons within the NAc promote NREM⁵³. As such, lesions within the NAc have eliminated wake-promoting effects of modafinil, a weak dopamine reuptake inhibitor and commonly used stimulant for excessive daytime sleepiness in a number of conditions including narcolepsy⁵⁴. It is therefore likely that VC stimulation, leading to activation of VTApp/MFB and anterior thalamic radiations, yields behavioral increases in arousal indirectly via the modulation of connected structures (e.g. NAc, VTA) or directly via activation of the prefrontal cortex or the thalamus⁵⁵.

The neighboring BNST has additionally been associated with the activation of norepinephrine neurons within the locus coeruleus, lateral hypothalamus, and VTA, all wake-promoting regions^{18,20}. Stimulation within the BNST has led to the rapid state transition to sustained wakefulness⁵⁶. In addition, distinct connections between the BNST and lateral hypothalamus lead to hypocretin/orexin expression and may be play a role in driving opposing emotional states^{20,57}. An alternative mechanism of VC stimulation therefore includes the activation of neighboring, wake-promoting subcortical structures, i.e. the BNST, included in the theoretical volume of activated tissue.

In our study, SGC stimulation demonstrated a moderate response in arousal, which may relate to its connectivity to structures involved in visceromotor activity and recent findings demonstrating an association with autonomic arousal^{58,59}. The arousal response within the SGC was overall less robust, which may relate to the variability in the identified fiber tracts recruited based on electrode positioning between the two patients (FIG. 1B). Finally, the OFC behavioral responses were also less robust, which may relate to the relatively large territory of the OFC and the associated variation across participants in electrode location and stimulation, spanning the medial to lateral OFC. Connectivity analysis revealed fibers corresponding to a branch of the anterior thalamic radiations, i.e., inferior thalamic peduncle, associated with effective OFC stimulation sites in two of the three patients and may also play a role in the modulation of arousal via the direct activation of thalamus⁵⁵.

It is also possible that the heterogeneity observed in both regions reflect a more complex relationship with arousal, such as being state dependent³⁰. Given the heterogeneity of responses and tracts recruited, further studies with more patients are required to elucidate which specific fiber tract(s) associated with SGC and OFC stimulation promote wakefulness. Further studies are also required to assess the differences in arousal responses between contralateral regions, for which systematic mapping was limited by statistical power due to the nature of the study to advance toward candidate mood-specific stimulation targets.

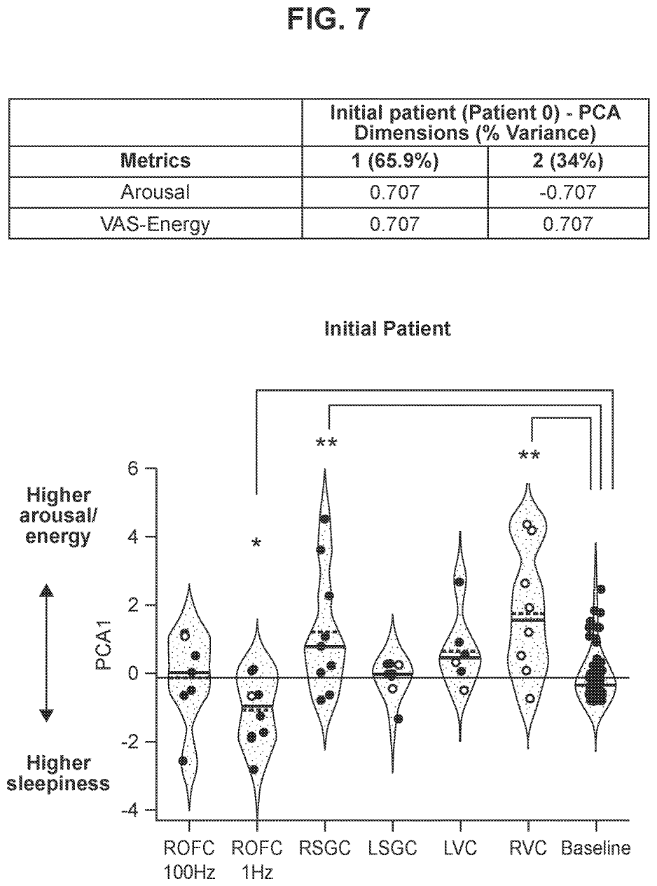

In addition to differential effects across regions, our stimulation responses indicate that the frequency of stimulation may have differential effects on arousal. While low and high frequency intracranial stimulation of OFC have been identified to yield specific antidepressant effects in a prior study³⁰, we demonstrate that 1 Hz OFC stimulation led to increased sleepiness, while 100 Hz OFC stimulation led to increased wakefulness. The differential frequency-dependent findings were similarly recapitulated with the initial patient with a subset of outcome measures (FIG. 7). Stimulation at low and high frequencies have been identified to selectively modulate excitatory and inhibitory neural populations^{60,61}, which likely gives rise to the variable clinical effects. Prior work has also demonstrated that low frequency stimulation is associated with presynaptic activation and high frequency activity is associated with synaptic depression^{62.63} The opposing effects on sleepiness induced by high and low frequency OFC stimulation suggest that stimulus parameter tuning will be essential for potential future therapeutic applications.

Although sleepiness is known to be associated with a decrease in higher frequency activity (alpha, beta, low gamma and high gamma) and an increase in delta and theta power in scalp EEG data⁶⁴⁻⁶⁶, the correlates of sleepiness within intracranial targets have not been systematically studied. Importantly, in our biomarker analysis, subjective sleepiness was found to correlate with specific spectral power features within the SGC, VC, amygdala, and OFC, providing further evidence for the distributed effects of the arousal network, including within corticolimbic regions^{8.9,67}. The most prominent biomarker features were increased in low and high gamma frequency activity, which were correlated with increased wakefulness and arousal, while increased lower frequency activity was associated with increased sleepiness, mirroring findings from surface EEG. Decreases in gamma activity with subjective sleepiness across all recorded regions suggest that perceived sleepiness is associated with broad changes in brain activity affecting both cortical and subcortical structures. These findings indicate that there are many brain regions that reflect sleepiness levels, which may also play a role in arousal and mood control. Furthermore, these findings are relevant for future efforts involving intracranial recordings and stimulation. In this regard, our recent work³⁰, revealing that the antidepressant effects of intracranial stimulation are dependent on arousal level, suggests that it may be important to characterize and control for arousal as part of optimal treatment delivery.

Our findings extend prior deep-brain stimulation studies in humans indicating that arousal level can be modulated with direct neural stimulation²⁵⁻²⁷ by expanding both the patient population and the target selection. Given that our study is performed in participants without significant neural injury or neurodegeneration, the participants were able to articulate their experiences and provide more sensitive outcome measures of sleepiness and energy to facilitate comparison between different stimulation targets. Prior studies of sleep-wake effects in patients with advanced PD and severe sleep disturbances also revealed that chronic low stimulation of the PPN led to decreased daytime sleepiness and improved sleep quality²⁹, whereas high frequency stimulation induced NREM sleep²⁸.

These findings reinforce frequency-dependent effects of stimulation, such that changes in stimulation frequency can yield opposing effects, as seen in our study with low and high frequency OFC stimulation. Finally, we highlight that a unique aspect of this study is that, unlike prior stimulus-response mapping work, primarily carried out in those with epilepsy, we had access to subjects without epilepsy undergoing a stimulation-mapping paradigm with bilateral corticolimbic implants (including the VC), thereby eliminating confounds related to anti-seizure medications or epileptiform activity.

This study has limitations. 1) Our findings in patients with treatment-resistant depression may not be generalizable to other patient populations. Further efforts to investigate arousal responses in patients without an underlying mood disorder will be important to assess any contribution from potentially altered emotional regulation networks. 2) The sample size in this study is small, limited to two patients. An important distinction from other intracranial mapping studies is that the two patients presented here underwent an intensive 10 d inpatient monitoring period with continuous recordings and comprehensive stimulation mapping, enabling a personalized, detailed, and robust evaluation.

In contrast, intracranial mapping studies that leverage patients undergoing monitoring EMU are limited to a small number of stimulation tests, spread across many patients (often due to the limited testing time). In our study, we therefore captured a large number of stimulation and biomarker recordings were collected per individual, leading to findings that were statistically powered within each patient. Future efforts to continue to expand this analysis to a larger cohort of patients is indicated and underway. 3) Finally, limited frequencies of stimulation were tested (1 Hz in OFC and 100 Hz in all other sites, i.e., frequencies optimized in depression studies); further exploration of different stimulation frequencies is required for the optimization for arousal responses and will be the subject of future work.

In summary, our work presents new evidence in human participants that stimulation of corticolimbic regions modulates subjective arousal level and that electrophysiologic biomarkers within corticolimbic sites correlate with changes in arousal. Three novel targets-VC, SGC, and OFC-were implicated in mediating arousal levels during wakefulness. Crucially, these findings indicate a broad overlap in the circuitry underlying mood and arousal, which may ultimately explain the strong behavioral and clinical links between mood and arousa^{6,7}. In addition, these findings open the door to new treatment targets and the consideration of therapeutic brain stimulation for sleep-wake disorders⁶⁸, such as idiopathic hypersomnia, a condition characterized by excessive daytime sleepiness. Future efforts to the map of brain regions involved in the extended arousal circuitry will be critical for the ongoing identification of novel treatment targets for the future application of neurostimulation for intractable sleep-wake disorders.

TABLE 1
Linear weights for summary dimension using
Principal Components Analysis (PCA)

| | Patient 1 - PCA Dimensions | | |
|---|---|---|---|
| Metrics | 1 (81.1%) | 2 (10.2%) | 3 (8.7%) |
| Arousal | 0.5454 | 0.7676 | 0.3366 |
| VAS-Energy | 0.5869 | −0.6365 | 0.5004 |
| Stanford Sleepiness Scale | −0.5984 | 0.0753 | 0.7977 |
| | Patient 2 - PCA Dimensions | | |
| Metrics | 1 (70.8%) | 2 (19.9%) | 3 (9.4%) |
| Arousal | 0.5151 | 0.8551 | −0.0582 |
| VAS-Energy | 0.6107 | −0.3185 | 0.725 |
| Stanford Sleepiness Scale | −0.6014 | 0.409 | 0.6863 |

### REFERENCES

1. Saper C B, Fuller P M. Wake-Sleep Circuitry: An Overview. Curr Opin Neurobiol. 2017; 44:186. doi: 10.1016/J.CONB.2017.03.021
2. Ashbrook L H, Krystal A D, Fu Y H, Ptáček L J. Genetics of the human circadian clock and sleep homeostat. Neuropsychopharmacology. 2020; 45 (1): 45-54. doi: 10.1038/S41386-019-0476-7
3. Wright K P, Lowry C A, leBourgeois M K. Circadian and wakefulness-sleep modulation of cognition in humans. Front Mol Neurosci. 2012; 0 (APRIL): 50. doi: 10.3389/FNMOL.2012.00050/BIBTEX
4. Konjarski M, Murray G, Lee V V, Jackson M L. Reciprocal relationships between daily sleep and mood: A systematic review of naturalistic prospective studies. Sleep Med Rev. 2018; 42:47-58. doi: 10.1016/J.SMRV.2018.05.005
5. Ptáček L J, Fu Y H, Krystal A D. Sleep and Mood: Chicken or Egg? Biol Psychiatry. 2016; 80 (11): 810-811. doi: 10.1016/J.BIOPSYCH.2016.09.012
6. Becker P M. Treatment of sleep dysfunction and psychiatric disorders. Curr Treat Options Neurol. 2006; 8 (5): 367-375. doi: 10.1007/S11940-006-0026-6
7. Alvaro P K, Roberts R M, Harris J K. A Systematic Review Assessing Bidirectionality between Sleep Disturbances, Anxiety, and Depression. Sleep. 2013; 36 (7): 1059-1068. doi: 10.5665/SLEEP.2810
8. Munn B R, Müller E J, Wainstein G, Shine J M. The ascending arousal system shapes neural dynamics to mediate awareness of cognitive states. Nat Commun 2021 121. 2021; 12 (1): 1-9. doi: 10.1038/s41467-021-26268-x
9. Petersen S E, Sporns O. Brain Networks and Cognitive Architectures. Neuron. 2015; 88 (1): 207-219. doi: 10.1016/j.neuron.2015.09.027
10. Sporns O. Network attributes for segregation and integration in the human brain. Curr Opin Neurobiol. 2013; 23 (2): 162-171. doi: 10.1016/j.conb.2012.11.015
11. Brown E N, Purdon P L, Van Dort C J. General Anesthesia and Altered States of Arousal: A Systems Neuroscience Analysis. dx.doi.org/101146/annurev-neuro-060909-153200.2011; 34:601-628. doi: 10.1146/ANNUREV-NEURO-060909-153200
12. Brown R E, Basheer R, McKenna J T, Strecker R E, McCarley R W. CONTROL OF SLEEP AND WAKEFULNESS. Physiol Rev. 2012; 92 (3): 1087. doi: 10.1152/PHYSREV.00032.2011
13. Feindel W, Gloor P. Comparison of electrographic effects of stimulation of the amygdala and brain stem reticular formation in cats. Electroencephalogr Clin Neurophysiol. 1954; 6 (C): 389-402. doi: 10.1016/0013-4694 (54) 90053-X
14. Jasper H, Pertuisset B, Flanigin H. Eeg and cortical electrograms in patients with temporal lobe seizures. Arch Neurol Psychiatry. 1951; 65 (3): 272-290. doi: 10.1001/ARCHNEURPSYC.1951.02320030009002
15. Llinás R R, Paré D. Of dreaming and wakefulness. Neuroscience. 1991; 44 (3): 521-535. doi: 10.1016/0306-4522 (91) 90075-Y
16. Oishi Y, Lazarus M. The control of sleep and wakefulness by mesolimbic dopamine systems. Neurosci Res. 2017; 118:66-73. doi: 10.1016/j.neures.2017.04.008
17. Oishi Y, Suzuki Y, Takahashi K, et al. Activation of ventral tegmental area dopamine neurons produces wakefulness through dopamine D2-like receptors in mice. Brain Struct Funct. 2017; 222 (6): 2907-2915. doi: 10.1007/s00429-017-1365-7
18. Eban-Rothschild A, Rothschild G, Giardino W J, Jones J R, De Lecea L. VTA dopaminergic neurons regulate ethologically relevant sleep-wake behaviors. Nat Neurosci. 2016; 19 (10): 1356-1366. doi: 10.1038/nn.4377
19. Luo Y J, Li Y D, Wang L, et al. Nucleus accumbens controls wakefulness by a subpopulation of neurons expressing dopamine D1 receptors. Nat Commun. 2018; 9 (1): 1-17. doi: 10.1038/s41467-018-03889-3
20. Giardino W J, Pomrenze M B. Extended Amygdala Neuropeptide Circuitry of Emotional Arousal: Waking Up on the Wrong Side of the Bed Nuclei of Stria Terminalis. Front Behav Neurosci. 2021; 15:6. doi: 10.3389/FNBEH.2021.613025/BIBTEX
21. Knutson B, Greer S M. Anticipatory affect: neural correlates and consequences for choice. Philos Trans R Soc B Biol Sci. 2008; 363 (1511): 3771. doi: 10.1098/RSTB.2008.0155
22. Russell J A. A circumplex model of affect. J Pers Soc Psychol. 1980; 39 (6): 1161-1178. doi: 10.1037/H0077714
23. Watson D, Tellegen A. Toward a Consensual Structure of Mood. Psychol Bull. 1985; 98 (2): 219-235. doi: 10.1037/0033-2909.98.2.219
24. Barrett L F. Discrete Emotions or Dimensions? The Role of Valence Focus and Arousal Focus. dx.doi.org/101080/026999398379574.2010; 12 (4): 579-599. doi: 10.1080/026999398379574
25. Schiff N D, Giacino J T, Kalmar K, et al. Behavioural improvements with thalamic stimulation after severe traumatic brain injury. Nature. 2007; 448 (7153): 600-603. doi: 10.1038/nature06041
26. Kundu B, Brock A A, Englot D J, Butson C R, Rolston J D. Deep brain stimulation for the treatment of disorders of consciousness and cognition in traumatic brain injury patients: a review. Neurosurg Focus. 2018; 45 (2): E14. doi: 10.3171/2018.5.FOCUS18168
27. Gummadavelli A, Kundishora A J, Willie J T, et al. Neurostimulation to improve level of consciousness in patients with epilepsy. Neurosurg Focus. 2015; 38 (6). doi: 10.3171/2015.3.FOCUS1535
28. Arnulf I, Ferraye M, Fraix V, et al. Sleep induced by stimulation in the human pedunculopontine nucleus area. Ann Neurol. 2010; 67 (4): 546-549. doi: 10.1002/ANA.21912
29. Peppe A, Pierantozzi M, Baiamonte V, et al. Deep Brain Stimulation of Pedunculopontine Tegmental Nucleus: Role in Sleep Modulation in Advanced Parkinson Disease Patients—One-Year Follow-Up. Sleep. 2012; 35 (12): 1637. doi: 10.5665/SLEEP.2234
30. Scangos K W, Makhoul G S, Sugrue L P, Chang E F, Krystal A D. State-dependent responses to intracranial brain stimulation in a patient with depression. Nat Med. 2021; 27 (2): 229-231. doi: 10.1038/s41591-020-01175-8
31. Scangos K W, Khambhati A N, Daly P M, et al. Closed-loop neuromodulation in an individual with treatment-resistant depression. Nat Med 2021 2710. 2021; 27 (10): 1696-1700. doi: 10.1038/s41591-021-01480-w
32. Horn A, Li N, Dembek T A, et al. Lead-DBS v2: Towards a comprehensive pipeline for deep brain stimulation imaging. Neuroimage. 2019; 184:293-316. doi: 10.1016/J.NEUROIMAGE.2018.08.068
33. Fonov V, Evans A, Mckinstry R, Almli C, Collins D. Unbiased nonlinear average age-appropriate brain templates from birth to adulthood. Neuroimage. 2009; 47: S102. doi: 10.1016/S1053-8119 (09) 70884-5
34. Horn A, Neumann W J, Degen K, Schneider G H, Kühn A A. Toward an electrophysiological “sweet spot” for deep brain stimulation in the subthalamic nucleus. Hum Brain Mapp. 2017; 38 (7): 3377-3390. doi: 10.1002/HBM.23594
35. Dembek T A, Barbe M T, Åström M, et al. Probabilistic mapping of deep brain stimulation effects in essential tremor. NeuroImage Clin. 2016; 13:164-173. doi: 10.1016/J.NICL.2016.11.019
36. Hoddes E, Zarcone V, Smythe H, Phillips R, Dement W C. Quantification of sleepiness: a new approach. Psychophysiology. 1973; 10 (4): 431-436. doi: 10.1111/J.1469-8986.1973.TB00801.X
37. Fan J M, Khambhati A N, Sellers K K, et al. Epileptiform discharges triggered with direct electrical stimulation for treatment-resistant depression: Factors that modulate risk and treatment considerations. Brain Stimul Basic, Transl Clin Res Neuromodulation. 2023; 0 (0). doi: 10.1016/J.BRS.2023.02.006
38. Mayberg H S, Riva-Posse P, Crowell A L. Deep Brain Stimulation for Depression: Keeping an Eye on a Moving Target. JAMA Psychiatry. 2016; 73 (5): 439-440. doi: 10.1001/JAMAPSYCHIATRY.2016.0173
39. Dougherty D D, Rezai A R, Carpenter L L, et al. A Randomized Sham-Controlled Trial of Deep Brain Stimulation of the Ventral Capsule/Ventral Striatum for Chronic Treatment-Resistant Depression. Biol Psychiatry. 2015; 78 (4): 240-248.
40. Rao V R, Sellers K K, Wallace D L, et al. Direct Electrical Stimulation of Lateral Orbitofrontal Cortex Acutely Improves Mood in Individuals with Symptoms of Depression. Curr Biol. 2018; 28 (24): 3893-3902.e4. doi: 10.1016/j.cub.2018.10.026
41. Malone D A, Dougherty D D, Rezai A R, et al. Deep Brain Stimulation of the Ventral Capsule/Ventral Striatum for Treatment-Resistant Depression. Biol Psychiatry. 2009; 65 (4): 267-275. doi: 10.1016/J.BIOPSYCH.2008.08.029
42. Schlaepfer T E, Bewernick B H, Kayser S, Mädler B, Coenen V A. Rapid Effects of Deep Brain Stimulation for Treatment-Resistant Major Depression. Biol Psychiatry. 2013; 73 (12): 1204-1212. doi: 10.1016/J.BIOPSYCH.2013.01.034
43. Bergfeld I O, Mantione M, Hoogendoorn MLC, et al. Deep brain stimulation of the ventral anterior limb of the internal capsule for treatment-resistant depression. JAMA Psychiatry. 2016; 73 (5): 456-464. doi: 10.1001/jamapsychiatry.2016.0152
44. Mayberg H S, Riva-Posse P, Crowell A L. Deep brain stimulation for depression: Keeping an eye on a moving target. JAMA Psychiatry. 2016; 73 (5): 439-440. doi: 10.1001/jamapsychiatry.2016.0173
45. Greenberg B D, Gabriels L A, Malone D A, et al. Deep brain stimulation of the ventral internal capsule/ventral striatum for obsessive-compulsive disorder: worldwide experience. Mol Psychiatry 2010 151. 2008; 15 (1): 64-79. doi: 10.1038/mp.2008.55
46. Baldermann J C, Melzer C, Zapf A, et al. Connectivity Profile Predictive of Effective Deep Brain Stimulation in Obsessive-Compulsive Disorder. Biol Psychiatry. 2019; 85 (9): 735-743. doi: 10.1016/J.BIOPSYCH.2018.12.019
47. Li N, Baldermann J C, Kibleur A, et al. A unified connectomic target for deep brain stimulation in obsessive-compulsive disorder. Nat Commun. 2020; 11 (1). doi: 10.1038/S41467-020-16734-3
48. Coenen V A, Bewernick B H, Kayser S, et al. Superolateral medial forebrain bundle deep brain stimulation in major depression: a gateway trial. Neuropsychopharmacol 2019 447. 2019; 44 (7): 1224-1232. doi: 10.1038/s41386-019-0369-9
49. Schlaepfer T E, Bewernick B H, Kayser S, Hurlemann R, Coenen V A. Deep brain stimulation of the human reward system for major depression—rationale, outcomes and outlook. Neuropsychopharmacology. 2014; 39 (6): 1303-1314. doi: 10.1038/NPP.2014.28
50. Coenen V A, Schlaepfer T E, Sajonz B, et al. Tractographic description of major subcortical projection pathways passing the anterior limb of the internal capsule. Corticopetal organization of networks relevant for psychiatric disorders. Neurolmage Clin. 2020; 25. doi: 10.1016/J.NICL.2020.102165
51. Haber S N, Yendiki A, Jbabdi S. Four Deep Brain Stimulation Targets for Obsessive-Compulsive Disorder: Are They Different? Biol Psychiatry. 2021; 90 (10): 667-677. doi: 10.1016/J.BIOPSYCH.2020.06.031
52. Dahan L, Astier B, Vautrelle N, Urbain N, Kocsis B, Chouvet G. Prominent burst firing of dopaminergic neurons in the ventral tegmental area during paradoxical sleep. Neuropsychopharmacology. 2007; 32 (6): 1232-1241. doi: 10.1038/S J.Npp.1301251
53. Oishi Y, Xu Q, Wang L, et al. Slow-wave sleep is controlled by a subset of nucleus accumbens core neurons in mice. Nat Commun 2017 81. 2017; 8 (1): 1-12. doi: 10.1038/s41467-017-00781-4
54. Qiu M H, Liu W, Qu W M, Urade Y, Lu J, Huang Z L. The Role of Nucleus Accumbens Core/Shell in Sleep-Wake Regulation and their Involvement in Modafinil-Induced Arousal. PLOS One. 2012; 7 (9). doi: 10.1371/JOURNAL.PONE.0045471
55. Redinbaugh M J, Phillips J M, Kambi N A, et al. Thalamus Modulates Consciousness via Layer-Specific Control of Cortex. Neuron. 2020; 106 (1): 66-75.e12. doi: 10.1016/J.NEURON.2020.01.005
56. Kodani S, Soya S, Sakurai T. Optogenetic Manipulation of Neural Circuits During Monitoring Sleep/wakefulness States in Mice. J Vis Exp. 2019; 2019 (148). doi: 10.3791/58613
57. Giardino W J, Eban-Rothschild A, Christoffel D J, Li S Bin, Malenka R C, de Lecea L. Parallel circuits from the bed nuclei of stria terminalis to the lateral hypothalamus drive opposing emotional states. Nat Neurosci 2018 218. 2018; 21 (8): 1084-1095. doi: 10.1038/s41593-018-0198-x
58. Rudebeck P H, Putnam P T, Daniels T E, et al. A role for primate subgenual cingulate cortex in sustaining autonomic arousal. Proc Natl Acad Sci USA. 2014; 111 (14): 5391-5396. doi: 10.1073/PNAS.1317695111/-/DCSUPPLEMENTAL
59. Alexander L, Wood C M, Gaskin P L R, et al. Over-activation of primate subgenual cingulate cortex enhances the cardiovascular, behavioral and neural responses to threat. Nat Commun 2020 111. 2020; 11 (1): 1-14. doi: 10.1038/s41467-020-19167-0
60. Mahmud M, Vassanelli S. Differential Modulation of Excitatory and Inhibitory Neurons during Periodic Stimulation. Front Neurosci. 2016; 10 (FEB). doi: 10.3389/FNINS.2016.00062
61. Lee K Y, Bae C, Lee D, et al. Low-intensity, Kilohertz Frequency Spinal Cord Stimulation Differently Affects Excitatory and Inhibitory Neurons in the Rodent Superficial Dorsal Horn. Neuroscience. 2020; 428:132-139. doi: 10.1016/J.NEUROSCIENCE.2019.12.031
62. Varela J A, Song S, Turrigiano G G, Nelson S B. Differential Depression at Excitatory and Inhibitory Synapses in Visual Cortex. J Neurosci. 1999; 19 (11): 4293. doi: 10.1523/JNEUROSCI.19-11-04293.1999
63. Liu L D, Prescott I A, Dostrovsky J O, Hodaie M, Lozano A M, Hutchison W D. Frequency-dependent effects of electrical stimulation in the globus pallidus of dystonia patients. J Neurophysiol. 2012; 108 (1): 5-17.
64. Hubbard J, Gent T C, Hoekstra M M B, et al. Rapid fast-delta decay following prolonged wakefulness marks a phase of wake-inertia in NREM sleep. Nat Commun 2020 111. 2020; 11 (1): 1-16. doi: 10.1038/s41467-020-16915-0
65. Åkerstedt T, Gillberg M. Subjective and Objective Sleepiness in the Active Individual. dx.doi.org/103109/00207459008994241.2009; 52 (1-2): 29-37.
66. De Gennaro L, Marzano C, Veniero D, et al. Neurophysiological correlates of sleepiness: A combined TMS and EEG study. Neuroimage. 2007; 36 (4): 1277-1287. doi: 10.1016/J.NEUROIMAGE.2007.04.013
67. Shine J M, Müller E J, Munn B, Cabral J, Moran R J, Breakspear M. Computational models link cellular mechanisms of neuromodulation to large-scale neural dynamics. Nat Neurosci 2021 246. 2021; 24 (6): 765-776. doi: 10.1038/s41593-021-00824-6
68. Venner A, Todd W D, Fraigne J J, et al. Newly identified sleep-wake and circadian circuits as potential therapeutic targets. Sleep. 2019; 42 (5). doi: 10.1093/SLEEP/ZSZ023

### Example 2: Intracranial Beta Activity is a Biomarker of Circadian and Stimulation-Induced Arousal in Obsessive Compulsive Disorder

Deep brain stimulation (DBS) has been investigated as therapy for treatment-resistant neuropsychiatric disorders, such as obsessive-compulsive disorder (OCD) [1] and major depressive disorder (MDD) [2]. However, side effects associated with increased arousal, such as insomnia and manic symptoms [3,4], can limit the efficacy of therapy. Manic symptoms, which include a decreased need for sleep, have been reported in up to 30-45% of individuals receiving anterior limb of internal capsule (ALIC) stimulation [3] and require urgent care. Insomnia is also a frequently reported adverse side effect [4], characterized by an inability to transition from wakefulness to sleep or frequent sleep disruptions. Furthermore, disruptions in sleep-wake function increase the risk of developing mania. While the underlying mechanisms of these side effects remain unclear, many of the activating effects of ALIC DBS can be reversed by decreasing the amplitude of stimulation [3]. These clinical observations indicate that ALIC DBS may recruit striato-limbic regions that modulate the baseline tone of arousal. Emerging literature suggests that incorporating the arousal state [5] and/or circadian rhythms into the stimulation approach may improve treatment and minimize related side effects [6]. A better understanding of the electrophysiological biomarkers of circadian and stimulation-related arousal may enable the development of adaptive ALIC DBS systems.

Cortical arousal manifests as shifts in activity towards higher frequencies. Increased beta activity measured from scalp EEG during wake and sleep has been associated with insomnia [7]. Furthermore, beta activity within intracranial EEG electrodes has been demonstrated to capture sleep-wake states across cortical sites [8]. In applications for Parkinson's disease, intracranial beta activity within subcortical regions shows circadian variability [6,9]. We therefore hypothesized that beta activity within striato-limbic regions may be a circadian biomarker of arousal.

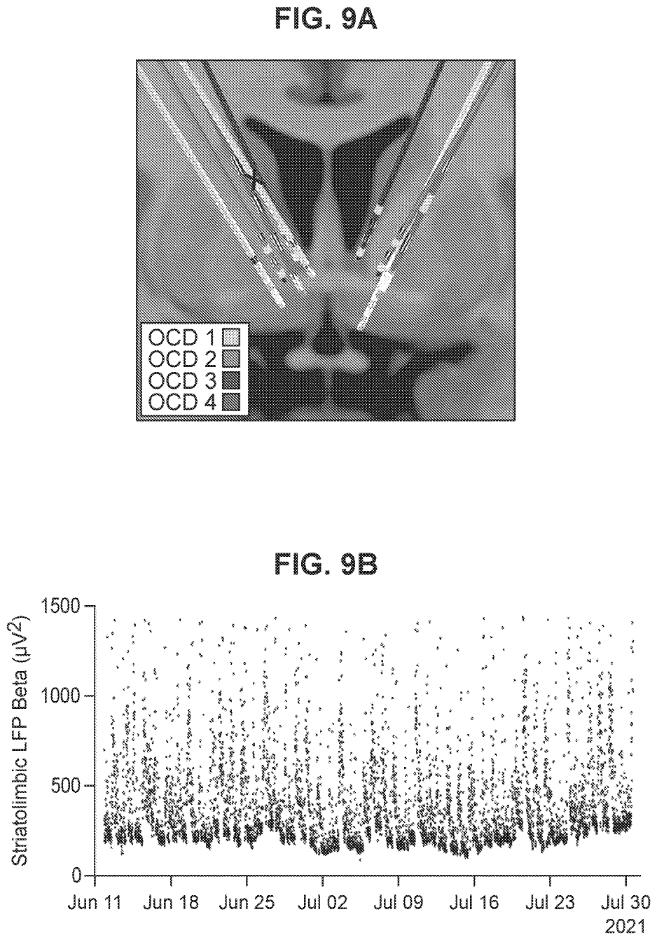

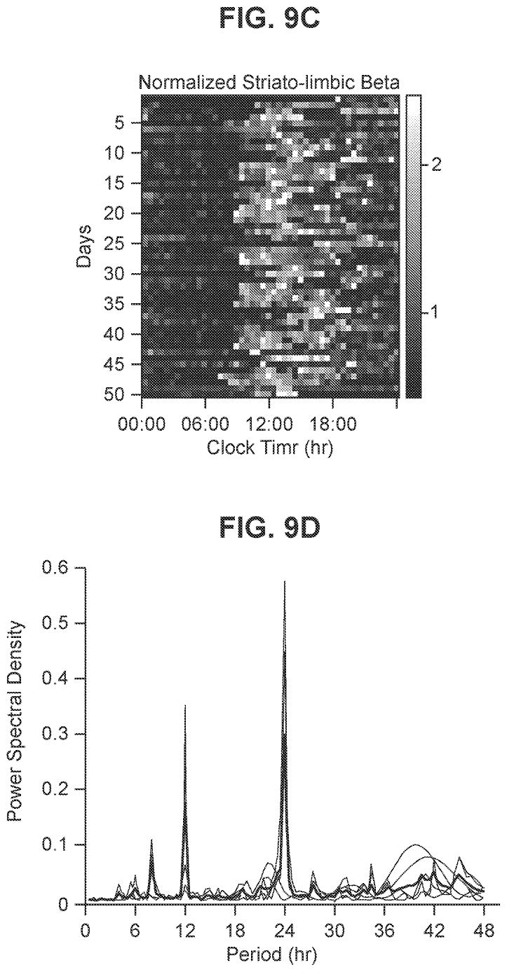

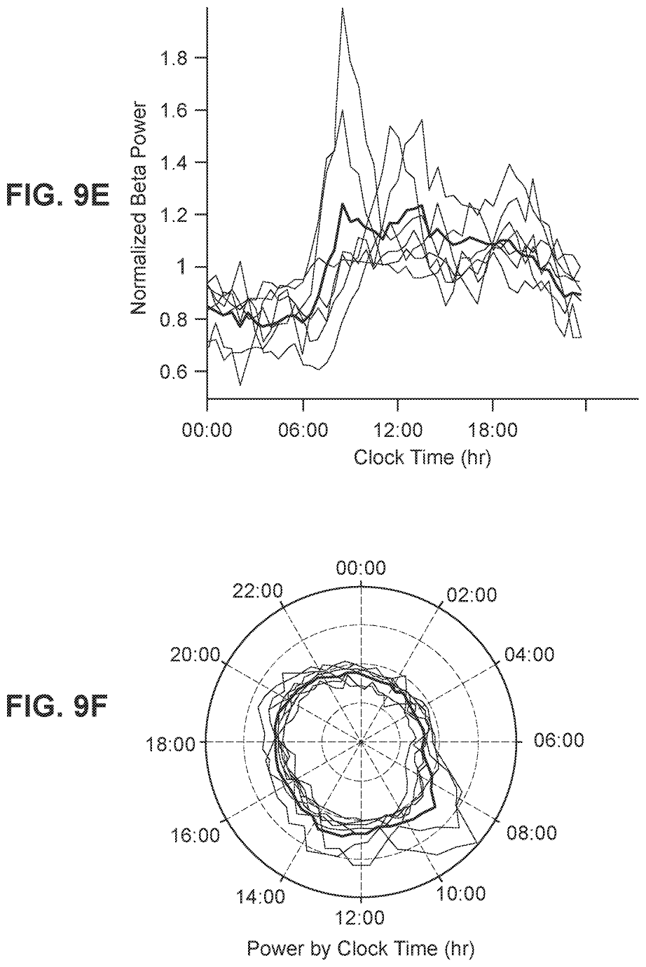

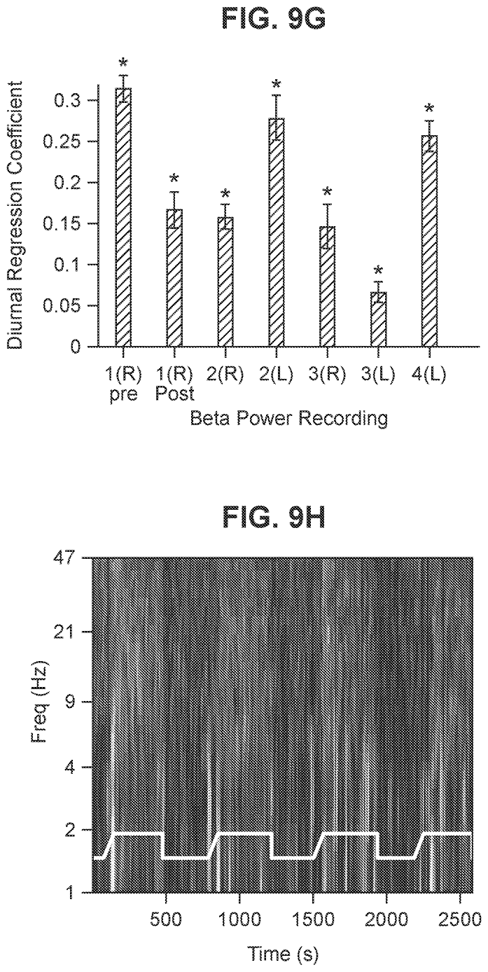

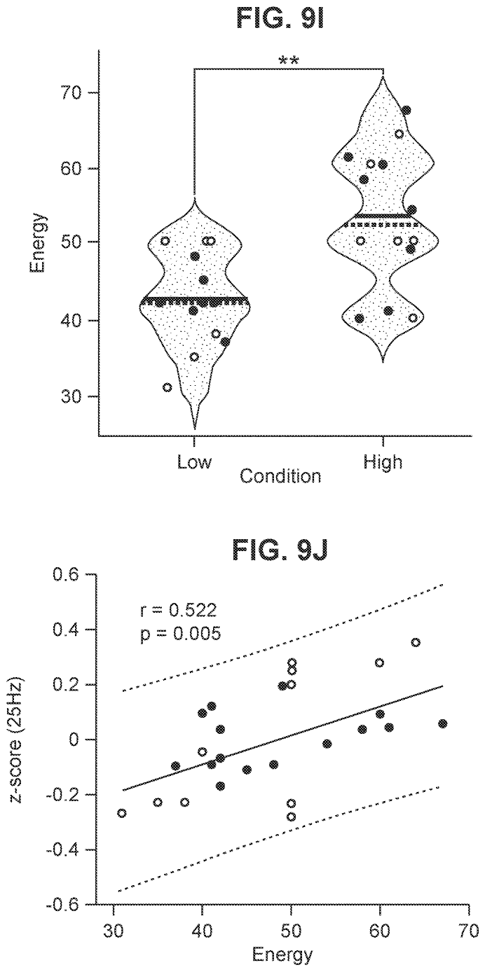

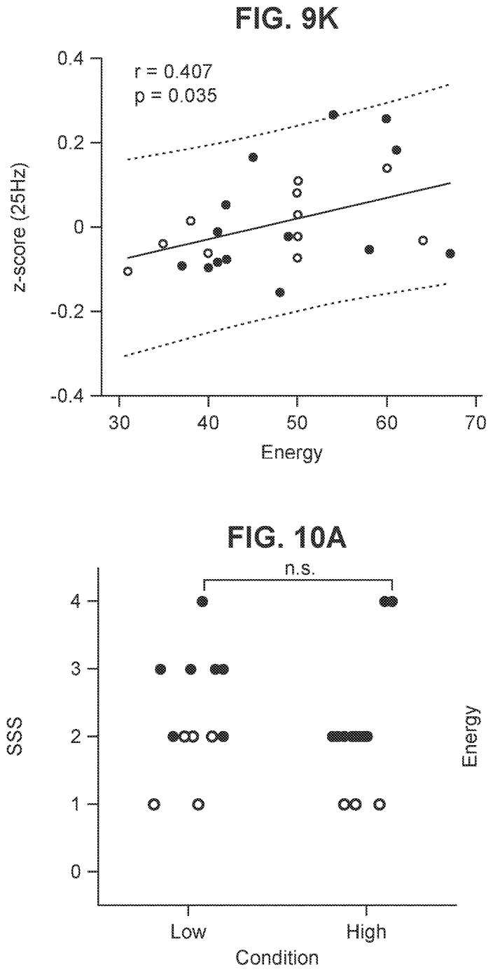

In a cohort of four treatment resistant OCD patients receiving DBS therapy, we aimed to better understand the mechanism of ALIC DBS-induced arousal by performing intracranial recordings from the neighboring striato-limbic region (FIG. 9A). We aimed to determine: 1) if striato-limbic beta activity is an electrophysiological biomarker of circadian variation of arousal and 2) whether DBS alters local striato-limbic beta in a manner that tracks with stimulation-induced arousal.

We recorded continuous band-limited spectral power within the beta frequency range in an ambulatory setting for 6-84 days (Table S1; Supplementary methods). These chronic recordings showed circadian fluctuations over long time scales, as in example patient OCD 1 (FIGS. 9B and 9C). The mean periodogram confirmed a circadian peak at 24 hrs (FIG. 9D). Across all subjects and recording sites, the detrended striato-limbic beta power depicted a significant change across a 24-h clock (FIGS. 9E and 9F; Tables S2 and S3 for individual statistical testing). Across leads, the sinusoidal fit was statistically significant (Table S2; p<0.0001 in all leads) and mean percent of variation explained by time of day was 11.1% [SD 3.73%] (FIG. 9G, Table S2). The detrended data was also converted to polar coordinates to quantify the non-uniform distribution of beta power (FIG. 9F, Table S3). The magnitude of resultant vectors was significant (Table S3; p<0.05) compared to shuffled data in all subjects except the left lead from OCD 3.

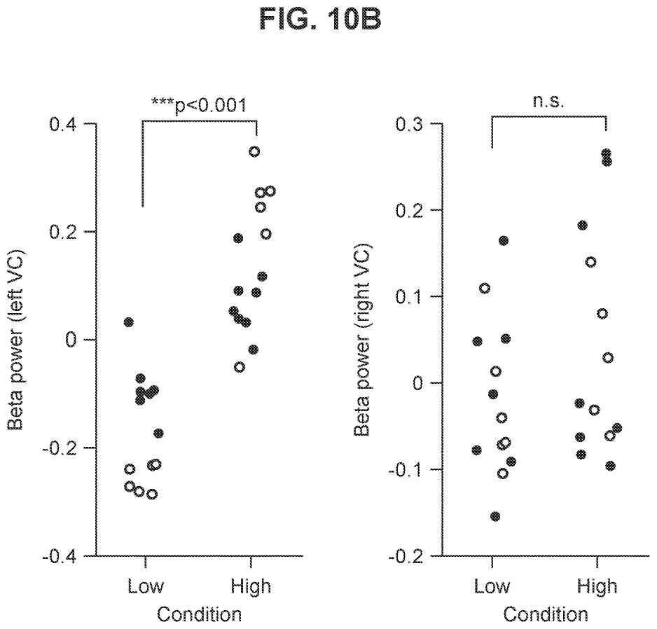

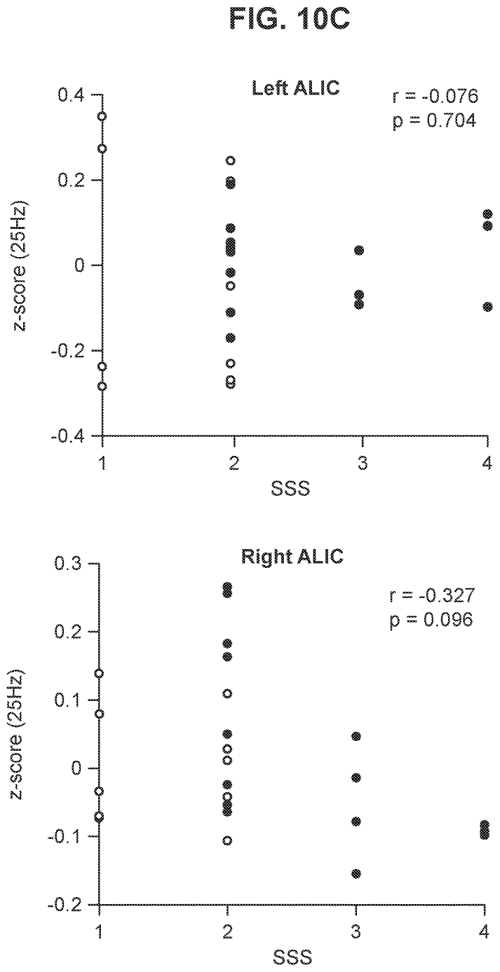

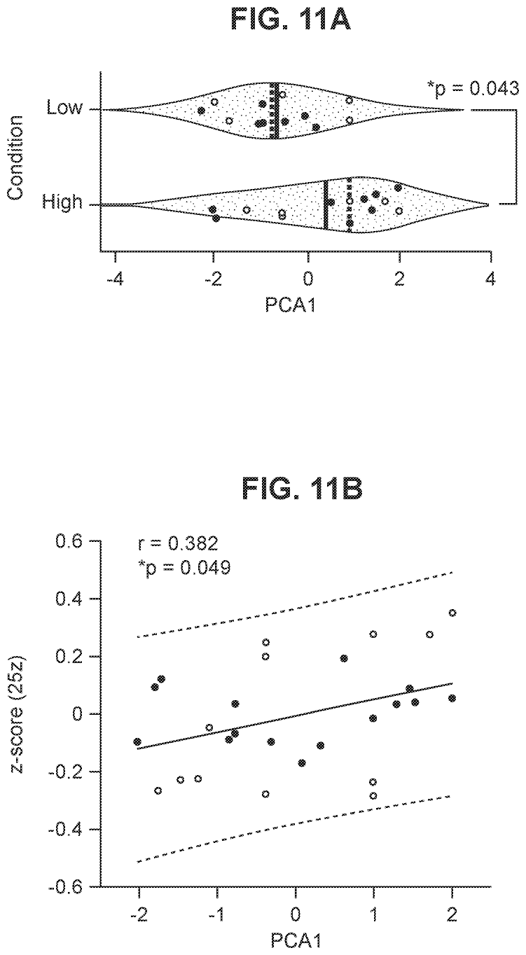

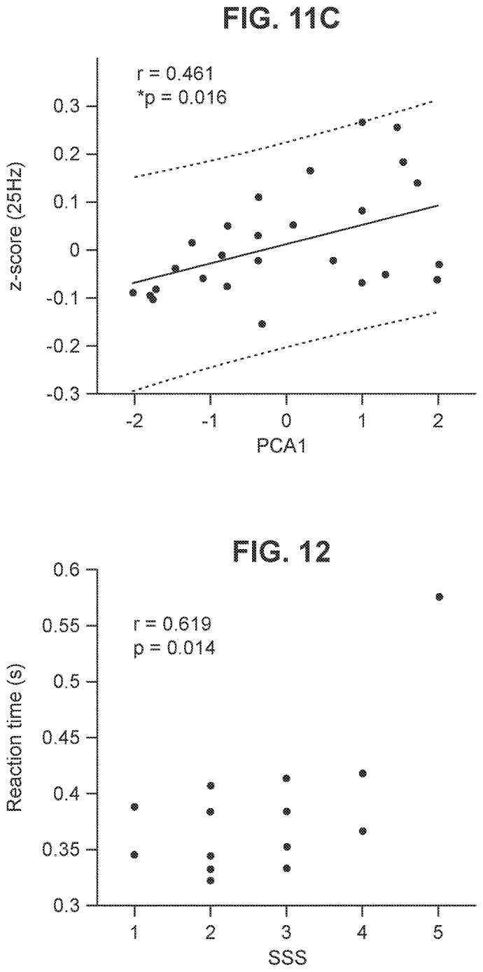

We next assessed the impact of changes in stimulation amplitude on arousal level in two of the subjects (OCD 1 and 4). Arousal levels were measured by the SSS and VAS-energy scales as stimulation was cycled from low to high amplitudes (Supplementary methods). Spectrograms were computed from LFP recordings from both the stimulation lead (example patient, left lead, FIG. 9H) and contralateral sensing lead. Across both subjects, an increase in stimulation amplitude was associated with an increase in subjective energy levels (two-sample Wilcoxon rank sum test, p=0.006; FIG. 9I), but not sleepiness levels (FIG. 10A, p=0.348). The linear combination of the two behavioral responses represented by the dominant principal component remained statistically significant with respect to stimulation amplitude (p=0.043; FIG. 11A). Increased stimulation amplitude corresponded to an acute increase in beta spectral power in the stimulation lead (FIG. 10B, left panel, p<0.001), but not the sensing lead (FIG. 10B, right panel). Furthermore, DBS-induced changes in arousal measured with the VAS-energy showed significant positive correlation with striato-limbic beta power in both the stimulation lead (FIG. 9J, Pearson's correlation, r=0.522, p=0.005) and sensing lead (FIG. 9K, r=0.407, p=0.035). Incorporating SSS levels in tandem with the VAS-energy as a combined measure of arousal using the dominant principal component improved the correlation with beta power in the sensing lead (FIG. 11C, r=0.461, p=0.016), suggesting that both measures may capture relevant behavioral measures that reflect DBS-induced changes in spectral power.

To our knowledge, this is the first study characterizing the circadian variation of beta activity within the striato-limbic region. It is likely that other frequency bands may also reflect a circadian pattern [8]. Prior work has demonstrated that regions neighboring the ALIC, such as the BNST and striatum, have key roles in circadian regulation [10], which may explain the strong circadian variation. Arousal effects stemming from ALIC stimulation may be related to local effects on striato-limbic regions. Prior studies have suggested that ALIC DBS can release dopamine [11], which could help sustain states of wakefulness. Alternatively, stimulation of white matter fibers may indirectly activate connected regions. For example, the anterior thalamic radiations connect the prefrontal cortex to the thalamus [12].

Our study demonstrates two key features of DBS targeting the ALIC region that may enable an adaptive, closed-loop paradigm to account for arousal effects: a biomarker and a tunable parameter of stimulation that modulates arousal. Incorporation of arousal effects into a closed-loop DBS system may mitigate stimulation-induced hyperarousal side effects, including sleep disruption and manic symptoms. In this frame-work, the striato-limbic beta biomarker may be used as a feedback signal to tailor stimulation amplitude to natural circadian variation of arousal. For instance, decreasing stimulation when excessive beta activity is detected during nighttime hours may prevent nighttime hyperarousal, which contributes to insomnia and mania. Furthermore, primary sleep-wake disorders, such as excessive daytime sleepiness, may also be treated with closed-loop approaches that promote natural circadian variation of arousal.

### REFERENCES

[1] Denys D, Graat I, Mocking R, et al. Efficacy of deep brain stimulation of the ventral anterior limb of the internal capsule for refractory obsessive-compulsive disorder: a clinical cohort of 70 patients. Am J Psychiatr 2020; 177 (3): 265-71.
[2] Dougherty D D, Rezai A R, Carpenter L L, et al. A randomized sham-controlled trial of deep brain stimulation of the ventral capsule/ventral striatum for chronic treatment-resistant depression. Biol Psychiatr 2015; 78 (4): 240-8.
[3] Widge A S, Licon E, Zorowitz S, et al. Predictors of hypomania during ventral capsule/ventral striatum deep brain stimulation. J Neuropsychiatry Clin Neurosci 2016; 28 (1): 38-44.
[4] Alonso P, Cuadras D, Gabri\ddot{ }els L, et al. Deep brain stimulation for obsessive-compulsive disorder: a meta-analysis of treatment outcome and predictors of response. PLOS One 2015; 10 (7): e0133591.
[5] Scangos K W, Makhoul G S, Sugrue L P, Chang E F, Krystal A D. State-dependent re-sponses to intracranial brain stimulation in a patient with depression. Nat Med 2021; 27 (2): 229-31.
[6] Gilron R, Little S, Wilt R, Perrone R, Anso J, Starr P A. Sleep-aware adaptive deep brain stimulation control: chronic use at home with dual independent linear discriminate detectors. Front Neurosci 2021; 15:732499.
[7] Zhao W, Van Someren E J W, Li C, et al. EEG spectral analysis in insomnia disorder: a systematic review and meta-analysis. Sleep Med Rev 2021; 59 (2): 101457.
[8] Fan J M, Lee A M, Sellers K K, et al. Intracranial electrical stimulation of cortico-limbic sites modulates arousal in humans. Brain Stimul 2023; 16 (4): 1072-82.
[9] Yin Z, Ma R, An Q, et al. Pathological pallidal beta activity in Parkinson's disease is sustained during sleep and associated with sleep disturbance. Nat Commun 2023; 14 (1): 5434.
[10] Giardino W J, Eban-Rothschild A, Christoffel D J, Li S Bin, Malenka R C, de Lecea L. Parallel circuits from the bed nuclei of stria terminalis to the lateral hypothalamus drive opposing emotional states. Nat Neurosci 2018; 21 (8): 1084-95.
[11] Figee M, De Koning P, Klaassen S, et al. Deep brain stimulation induces striatal dopamine release in obsessive-compulsive disorder. Biol Psychiatr 2014; 75 (8): 647-52.
[12] Li N, Baldermann J C, Kibleur A, et al. A unified connectomic target for deep brain stimulation in obsessive-compulsive disorder. Nat Commun 2020; 11 (1).

### Supplementary Methods

### Subject Cohort

Four subjects with a history of severe, treatment-refractory OCD, who were treated with DBS targeting the bilateral ALIC and surrounding bed nucleus of the stria terminalis (BNST) using the Percept PC system (3387, 3391, or B33015; Medtronic, Minneapolis, MN), were included in this study. The Percept PC is the first clinically approved system to simultaneously stimulate and record local field potential (LFP). None of the subjects had previously been diagnosed with a sleep-wake disorder nor were they on medications for sleep disruption. All experimental protocols were approved by the UCSF Committee for Human Research, and written informed consent was obtained in all subjects.

### Electrode Placement

All subjects had previously undergone a 3T brain magnetic resonance image (MRI) and stereotactic-guided placement of DBS electrodes targeting the ALIC. Post-operative computed tomography (CT) scans were performed to confirm electrode localization. To visualize electrode placement across all four subjects in a single figure, pre-operative MRI and post-operative CT scans were co-registered and normalized to a standardized Montreal Neurological Institute (MNI) template (FIG. 1A). Electrode localization was then visualized using the open-access toolbox Lead-DBS⁴³. We note that for one patient, OCD1, the right lead required re-positioning for clinical indications; thus, two recording periods before and after the re-positioning were investigated, as denoted by pre and post.

### Chronic Timeline Power Recordings

Chronic ambulatory recordings were obtained using the Percept System BrainSense™ Timeline Mode. In this mode, mean power was recorded every 10 min within a chosen 5 Hz band from contacts that were not delivering stimulation. The center bands of three patients were set to 25.4 Hz, and the center frequency for an additional patient was set to 32.3 Hz (Table 1 s). Analyzed recording duration ranged from 6 to 84 days. Chronic stimulation paradigms were determined by clinical need. Thus, stimulation was at times enabled or disabled in the contralateral lead from the recording site. Recording was never performed on the same side as stimulation to minimize the impact of stimulation-related artifacts.

### Chronic Power Recording Analysis and Statistical Testing

Averaged spectral power within the narrow frequency band was evaluated for circadian rhythms. Spectral power was averaged within non-overlapping 30-minute time bins. Outliers were defined as power greater than 3 standard deviations over the mean of each recording and omitted from the data set. Omitted outliers were replaced using linear interpolation. Periodograms were calculated by the periodogram function in Matlab, A rectangular filter window was used for maximum PSD resolution. For subjects OCD2 and OCD3, extended epochs were recorded during which stimulation was turned off in both leads; thus, periodograms and quantification of circadian variability could be calculated in both leads.

Averaged spectral power was detrended by normalizing each day's data to that of its mean to account for long-term amplitude fluctuations. For each lead, preprocessed spectral power data was plotted against 24-hour clock time. A sine curve with 24-hour period was then fitted to the data (Eq. 1).

$$Normalized Beta Power = A * sin ( \frac{2 π}{24} ( t - B ) ) + 1 \qquad Eq . 1$$

The data was converted to polar coordinates, with polar angle of 2π corresponding to 24 hours, to calculate the resultant vector of each recording. To test the statistical significance, permutation testing was performed, in which the time series was shuffled and transformed into polar coordinates 1000 times to generate a null distribution of resultant vector magnitudes that was compared to the true resultant vectors. The percent of variance explained by time of day in the detrended data was estimated by the coefficient of determination, R², of the sinusoidal fit. Code for data visualization was adapted from the Circa Diem MATLAB toolbox [46].

### Stimulation Cycling Paradigm for Assessing Amplitude Response

A cyclical stimulation paradigm was performed in a subset of subjects (OCD1 and OCD4), who were amenable to stimulation amplitude changes, to evaluate the effect on subjective arousal levels. The cyclical stimulation paradigm was performed in clinic using the BrainSense™ Streaming mode. Specifically, continuous stimulation was performed over 40-50 minutes, during which time stimulation amplitude was cycled from the subject's baseline stimulation amplitude to an increased amplitude (i.e., 1 and 2.5 mA above baseline for OCD 1 and 4, respectively, as determined by tolerability). Due to the clinical and long-term nature of stimulation for each subject, the stimulation parameter optimized for treatment of their condition was used as the baseline condition. The baseline stimulation condition consisted of monophasic, 130 Hz stimulation of the left ALIC, 100 μs pulse width stimulation for both patients at 5 mA for OCD1 and 6 mA OCD4.

### Behavioral Measures

To quantify the subjective change in arousal level with stimulation amplitude changes, two behavioral surveys were administered. First, the subjects reported their energy using a visual-analog scale (VAS) of energy, which ranged from 0 (i.e. “not at all energetic”) to 100 (i.e. “very energetic”). Second, the subjects rated their sleepiness levels using the validated Stanford Sleepiness Scale (SSS)⁴⁴, a 7-point self-rated scale of sleepiness from 1 (“Feeling active, vital, alert or wide awake) to 7 (“No longer fighting sleep, sleep onset soon, having dream-like thoughts”). Surveys were administered at 2 and 5 minutes following a change in stimulation amplitude.

### Behavioral and Electrophysiologic Responses to Stimulation Cycling

During the cyclical stimulation paradigm, LFP was obtained in bipolar configuration, i.e. bipolar contacts for OCD1 1 and 3 (both left and right leads) and OCDA 0 and 2 (both right and left leads). On the stimulation leads, the recording contacts flanked the monopolar stimulation contact. LFP was sampled at 250 Hz (i.e., the maximum sampling rate by the DBS device). Spectral power was computed using the Hilbert transform. To determine the change over time with alternating stimulation conditions, the spectral power was z-scored to the initial baseline, as defined before the first high stimulation condition. Averaged z-scored, beta power centered over 25 Hz (i.e. between 22-27 Hz) was used as a proxy for narrow band beta power and was averaged across the 120 s time bin corresponding to each behavioral score. Behavioral measures for individual SSS and VAS-E scores were assessed during high and low stimulation intensities. In addition, a composite summary score was computed that combined all SSS and VAS-E responses on a single dimension, as determined by dimension 1 of a principal components analysis (PCA1). Specifically, we used the pca function in Matlab with the input of all observations capturing both the SSS and VAS-E, and subsequently evaluated the first PCA dimension conferring the highest component variance. Statistical testing to compare behavior and beta power in low and high stimulation conditions was performed using a two-sample Wilcoxon rank sum test to compare categorical measures. In addition, correlations between behavioral scores and corresponding beta power were computed by Pearson's correlation.

TABLE S1
Long-term LFP recording characteristics

| | Subject (Lead laterality of the sensing lead) | | | | | | |
|---|---|---|---|---|---|---|---|
| | OCD | OCD | | | | | |
| | 1 (R, | 1 (R, | OCD | OCD | OCD | OCD | OCD |
| | pre‡) | post) | 2 (R) | 2 (L) | 3 (R) | 3 (L) | 4 (L) |
| Center Frequency (Hz) | 25.39 | 25.39 | 25.39 | 25.39 | 25.39 | 25.39 | 32.23 |
| Sensing channels | 1/3 | 0/2 | 0/2 | 0/2 | 0/2 | 0/3 | 0/2 |
| (bipolar contacts) | | | | | | | |
| Analyzed LFP Duration | 84 | 12 | 74 | 35 | 6 | 6 | 28 |
| (days) | | | | | | | |
| Electrode Lead Model* | 3387 | B33015 | 3387 | 3387 | B33015 | B33015 | 3391 |
| Stimulation Amplitude | 6-7.5 | 0 | 0, 7, 9 | 0 | 0 | 0 | 6.5 |
| of active/contralateral | | | | | | | |
| lead (mA) | | | | | | | |
| Stimulating Contacts of | C+/2− | N/A | C+/2− | N/A | N/A | N/A | C+/1− |
| active/contralateral lead | | | | | | | |
| Impedance (Ω)^{†} | 1453, 1289 | 2032 | 2489, 1887 | 2600 | 3694, 3803, 3667 | 1801 | 2016 |

*Lead model used in the Percept PC system (Medtronic, Minneapolis, MN)
^{†}Impedance of electrode channels measured most recently. For OCD 1 (R, pre) and OCD 2(R) two impedance recordings were measured because the recording period was not continuous. For OCD 3 (L), three impedances were measured from three directional Sensight contacts, but the LFP measurement is a combined single-channel recording.
‡Pre and post qualifiers refer to time before and after lead repositioning in the same patient.

TABLE S2
Sinusoidal analysis of detrended data across patients

| | Subject (Lead) | | | | | | |
|---|---|---|---|---|---|---|---|
| | OCD | OCD | | | | | |
| | 1 (R, | 1 (R, | OCD | OCD | OCD | OCD | OCD |
| | pre) | post) | 2 (R) | 2 (L) | 3 (R) | 3 (L) | 4 (L) |
| Coefficient of | 0.108 | 0.116 | 0.039 | 0.106 | 0.123 | 0.121 | 0.165 |
| Determination, R² | | | | | | | |
| Sinusoidal Amplitude | 0.318 | 0.168 | 0.160 | 0.282 | 0.148 | 0.068 | 0.2560 |
| Term A [95% CI] | [0.301, | [0.146, | [0.145, | [0.255, | [0.121, | [0.055, | [0.241, |
| | 0.334] | 0.191] | 0.175] | 0.309] | 0.175] | 0.080] | 0.278] |
| Sinusoidal Phase term | 10.040 | 8.938 | 4.377 | 5.661 | 11.181 | 10.184 | 9.933 |
| B [95% CI] | [9.845, | [7.521, | [4.018, | [4.366, | [10.497, | [9.489, | [9.650, |
| | 10.238] | 10.351] | 4.740] | 6.960 | 11.865] | 10.875] | 10.200] |
| Sinusoidal Fit p-value | 1.648E−297 | 1.574E−46 | 3.601E−93 | 1.516E−121 | 1.674E−25 | 1.209E−24 | 5.187E−158 |

TABLE S3
Polar coordinate analysis of detrended data across patients

| | Subject (Lead) | | | | | | |
|---|---|---|---|---|---|---|---|
| | OCD | OCD | | | | | |
| | 1 (R, | 1 (R, | OCD | OCD | OCD | OCD | OCD |
| | pre) | post) | 2 (R) | 2 (L) | 3 (R) | 3 (L) | 4 (L) |
| Resultant Vector | 0.148 | 0.065 | 0.077 | 0.127 | 0.044 | 0.003 | 0.128 |
| Magnitude | | | | | | | |
| Resultant Vector Phase | −2.114 | −2.335 | 2.715 | 3.070 | −1.519 | 0.174 | −2.088 |
| (rad) | | | | | | | |
| Resultant Vector Phase | 15.924 | 15.080 | 10.372 | 11.726 | 18.198 | 0.663 | 16.023 |
| (hr) | | | | | | | |
| Shuffle test p-value | p < 0.001 | p < 0.001 | p < 0.001 | p < 0.001 | 0.049 | 1 | p < 0.001 |

### Example 3: Assessment of Whether Increased Energy/Wakefulness Translated into Improved Reaction Times in a Standardized Psychomotor Vigilance Test

We assessed whether increased energy/wakefulness translated into improved reaction times in a standardized psychomotor vigilance test. This is a well-known assessment that captures the reaction time as a surrogate of vigilance. First, we demonstrated that across all baseline PVTs vs. closest SSS survey, there is a positive, statistically significant relationship between reaction time (RT) and Stanford sleepiness score (SSS), indicating that that RT here reflects the degree of sleepiness (FIG. 12).

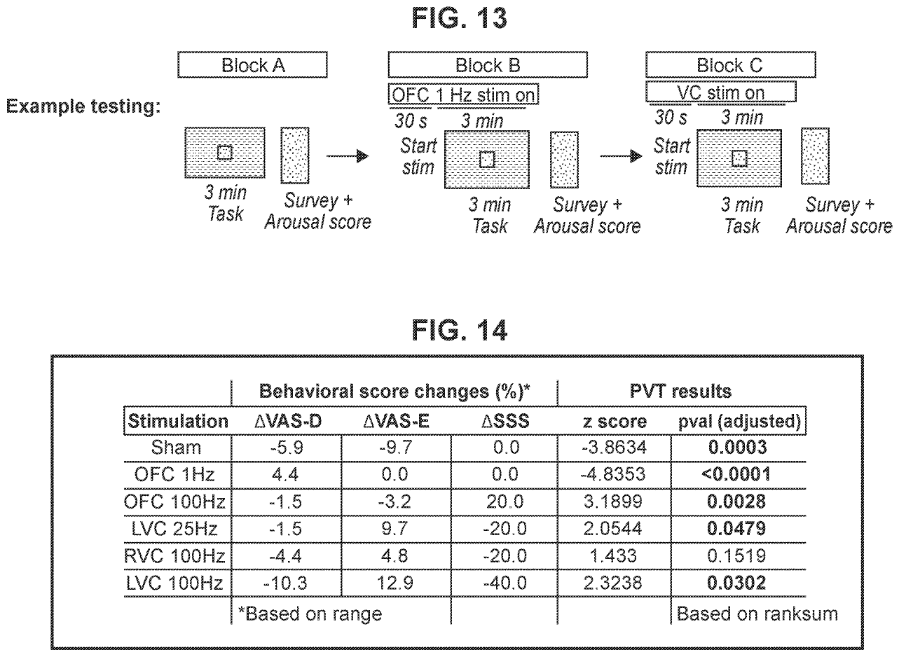

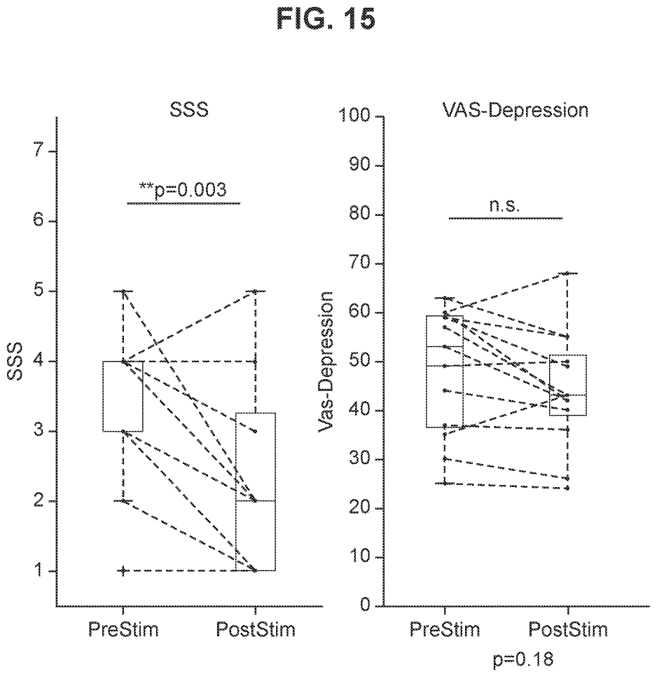

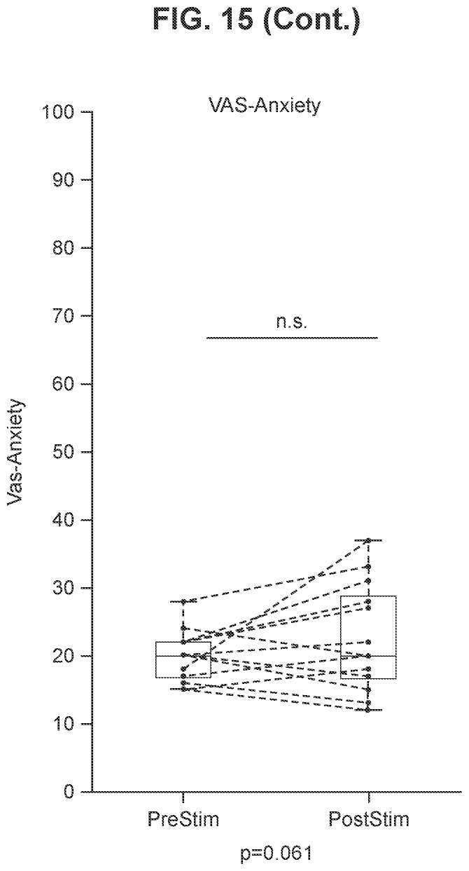

We next evaluated the effect of stimulation on reaction times in a patient (FIG. 13). We performed sequential stimulation and PVT testing in serial assessments, e.g., Block A-C/D, altering stimulation conditions to assess stimulation effects (FIG. 13). Initial findings demonstrate that stimulation of the OFC at 1 Hz increases the RT based on the PVT results (i.e. slows reaction time), while specific conditions of VC stimulation and OFC stimulation at 100 Hz decreases the RT (i.e. quickens the reaction time). In contrast, sham slows reaction time, which is not surprising given that persistent testing of the PVT is likely to lead decreased vigilance over time. This subject furthermore demonstrated that reduction of sleepiness (enhancement of wakefulness) is attainable in absence of altering other mood measures, such as depression and anxiety (FIG. 14). This was manifested below where we pulled together pre and post stimulation responses from an initial stimulation to VC stimulation. In this context, the patient reports comments such as “I think I feel the energy”, or is witnessed to go from the recumbent position with eyes closed to then getting up and opening her laptop.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of modulating sleep-wake levels in a subject, the method comprising applying electrical stimulation to a location in a ventral capsule (VC) region, an orbitofrontal cortex (OFC) region, or a subgenual cingulate (SGC) region of the subject using an electrode in a manner effective to modulate sleep-wake levels of the subject.

2. The method of claim 1, wherein the location in the VC region is in a right VC region, a left VC region, or a combination thereof.

3. The method of claim 1 or 2, wherein said applying the electrical stimulation comprises applying electrical stimulation at a frequency in a range from 70 Hz to 250 Hz such that wakefulness or energy of the subject is increased, or reaction time of the subject is decreased compared to wakefulness, energy, or reaction time of the subject before said applying the electrical stimulation.

4. The method of claim 1 or 2, wherein said applying the electrical stimulation comprises applying electrical stimulation at a frequency in a range of 0.5 Hz to 10 Hz such that sleepiness or reaction time of the subject is increased compared to sleepiness or reaction time of the subject before said applying the electrical stimulation.

5. The method of any one of claims 1-4, wherein said applying the electrical stimulation comprises applying electrical stimulation for 5 seconds to 120 seconds.

6. The method of any one of claims 1-5, wherein said applying the electrical stimulation comprises applying electrical stimulation intermittently with a rest period between stimulations.

7. The method of claim 6, wherein the rest period ranges from 10 seconds to 10 minutes.

8. The method of claim 7, wherein said applying the electrical stimulation comprises applying electrical stimulation intermittently with a stimulation period of 6 seconds and a rest period of 10 seconds, or a stimulation period of 30 seconds and a rest period of 2 minutes.

9. The method of any one of claims 1-8, further comprising mapping the VC region, the OFC region, or the SGC region of the subject to identify an optimal location for positioning the electrode for delivery of the electrical stimulation to modulate sleep-wake levels of the subject.

10. The method of any one of claims 1-9, wherein the electrode is placed on a surface of the VC region, the OFC region, or the SGC region.

11. The method of any one of claims 1-9, wherein the electrode is placed within the VC region, the OFC region, or the SGC region.

12. The system of any one of claims 1-11, wherein the electrode is a non-brain penetrating surface electrode array or a brain-penetrating electrode array.

13. The method of any one of claims 1-12, wherein the subject has a sleep-wake disorder, wherein applying the electrical stimulation treats the sleep-wake disorder.

14. The method of claim 13, wherein the sleep-wake disorder is hypersomnia, excessive daytime sleepiness, insomnia, sleep apnea, parasomnia, narcolepsy, restless leg syndrome, non-rapid eye movement (NREM) sleep arousal disorder, or rapid eye movement (REM) sleep behavior disorder.

15. The method of claim 14, wherein the hypersomnia is caused by idiopathic hypersomnia, narcolepsy, Kleine-Levin syndrome, Prader-Willi syndrome, Norrie disease, Niemann-Pick disease type C, myotonic dystrophy, fibromyalgia, chronic fatigue syndrome, lupus, rheumatoid arthritis, Morvan's syndrome, hypothyroidism, multiple sclerosis, encephalitis, Alzheimer's disease, Parkinson's disease, hydrocephalus, anemia, encephalitis lethargica, paraneoplastic syndrome, head trauma, or depression.

16. The method of claim 14, wherein the insomnia is caused by deep brain stimulation therapy to an anterior limb of internal capsule (ALIC) region of the subject.

17. The method of claim 16, wherein the subject has obsessive-compulsive disorder or major depressive disorder.

18. The method of any one of claims 1-17, wherein the electrical stimulation is applied unilaterally or bilaterally.

19. The method of any one of claims 1-18, further comprising assessing effectiveness of the electrical stimulation in said modulating the sleep-wake levels of the subject.

20. The method of claim 19, wherein said assessing comprises measuring a level of sleepiness or wakefulness of the subject using a visual-analog scale (VAS), a psychomotor vigilance test (PVT), a Likert scale, a Stanford Sleepiness Scale (SSS), a maintenance of wakefulness test (MWT), an Epworth sleepiness scale (ESS), a multiple sleep latency test (MSLT), or an Athens insomnia scale.

21. The method of any one of claims 1-20, further comprising monitoring sleepiness or wakefulness of the subject using actigraphy or polysomnography.

22. The method of any one of claims 1-21, further comprising monitoring wakefulness or sleepiness of the subject by recording a brain electrical signal associated with wakefulness or sleepiness of the subject, wherein the brain electrical signal is from a ventral capsule (VC) region, a subgenual cingulate (SGC) region, an amygdala region, an orbitofrontal cortex (OFC) region, or a striato-limbic region of the subject.

23. The method of any one of claims 1-22, wherein the brain electrical signal associated with wakefulness or sleepiness comprises alpha frequency, beta frequency, gamma frequency, delta frequency, or theta frequency neural oscillations.

24. The method of claim 23, wherein the gamma frequency neural oscillations are low gamma frequency neural oscillations in a range from 31 Hz to 81 Hz or high gamma frequency neural oscillations in a range from 81 Hz to 150 Hz.

25. The method of claim 23 or 24, further comprising measuring a relative spectral power level for the gamma frequency neural oscillations, wherein higher relative power of the gamma frequency neural oscillations is associated with lower sleepiness and higher wakefulness compared to lower relative power of the gamma frequency neural oscillations.

26. The method of any one of claims 22-25, wherein said applying electrical stimulation comprises applying electrical stimulation when the recorded brain electrical signal associated with wakefulness or sleepiness of the subject exceeds a threshold level.

27. The method of any one of claims 22-26, wherein the subject is monitored continuously or intermittently.

28. The method of any one of claims 1-27, wherein said applying electrical stimulation comprises applying non-invasive brain stimulation (NIBS) to the VC region, the OFC region, or the SGC region.

29. The method of any one of claims 1-28, wherein said applying electrical stimulation comprises applying non-invasive brain stimulation (NIBS) to a right VC region, a left VC region, or a combination thereof.

30. A closed-loop method for treating hypersomnia in a subject, the method comprising:
recording a brain electrical signal associated with sleepiness of the subject using a first electrode, wherein the brain electrical signal is from a ventral capsule (VC) region, a subgenual cingulate (SGC) region, an amygdala region, an orbitofrontal cortex (OFC) region, or a striato-limbic region, of the subject; and
applying electrical stimulation to a location in the VC region, the OFC region, or the SGC region of the subject using a second electrode in a manner effective to treat the hypersomnia in the subject when the electrical signal that is detected using the first electrode exceeds a threshold level.

31. The method of claim 30, wherein the location in the VC region is in a right VC region, a left VC region, or a combination thereof.

32. The method of claim 30 or 31, wherein said applying the electrical stimulation comprises applying electrical stimulation at a frequency in a range from 70 Hz to 250 Hz such that wakefulness or energy of the subject is increased compared to wakefulness or energy of the subject before said applying the electrical stimulation.

33. The method of any one of claims 30-32, wherein said applying the electrical stimulation comprises applying electrical stimulation for 5 seconds to 120 seconds.

34. The method of any one of claims 30-33, further comprising mapping the ventral capsule (VC) region, a subgenual cingulate (SGC) region, an amygdala region, an orbitofrontal cortex (OFC) region, or a striato-limbic region of the subject to identify an optimal location for positioning the first electrode for recording the brain electrical signal associated with sleepiness of the subject.

35. The method of any one of claims 30-34, further comprising mapping the VC region, the OFC region, or the SGC region of the subject to identify an optimal location for positioning the second electrode for delivery of the electrical stimulation to treat the hypersomnia of the subject.

36. The method of any one of claims 30-35, wherein the electrode is placed on a surface of the VC region, the OFC region, or the SGC region.

37. The method of any one of claims 30-35, wherein the electrode is placed within the VC region, the OFC region, or the SGC region.

38. The system of any one of claims 30-37, wherein the electrode is a non-brain penetrating surface electrode array or a brain-penetrating electrode array.

39. The method of any one of claims 30-38, further comprising using a control algorithm to automate said applying electrical stimulation when the brain electrical signal associated with sleepiness exceeds the threshold level.

40. The method of claim 39, wherein the control algorithm comprises a machine learning algorithm for classification of degree of sleepiness based on the brain electrical signal.

41. The method of claim 40, wherein the machine learning algorithm is a supervised machine learning algorithm.

42. The method of any one of claims 39-41, wherein the control algorithm further modulates one or more programmed stimulation parameters based on a level of power of the brain electrical signal.

43. The method of any one of claims 30-42, wherein the brain electrical signal associated with sleepiness comprises alpha frequency, beta frequency, gamma frequency, delta frequency, or theta frequency neural oscillations.

44. The method of claim 43, wherein the gamma frequency neural oscillations are low gamma frequency neural oscillations in a range from 31 Hz to 81 Hz or high gamma frequency neural oscillations in a range from 81 Hz to 150 Hz.

45. The method of claim 43 or 44, further comprising measuring a relative spectral power level for the gamma frequency neural oscillations, wherein higher relative power of the gamma frequency neural oscillations is associated with lower sleepiness and higher wakefulness compared to lower relative power of the gamma frequency neural oscillations.

46. The method of any one of claims 30-45, wherein the subject is monitored continuously or intermittently.

47. The method of any one of claims 30-46, wherein the electrical stimulation is applied unilaterally or bilaterally.

48. The method of any one of claims 30-47, wherein said applying electrical stimulation comprises applying non-invasive brain stimulation (NIBS) to the VC region, the OFC region, or the SGC region.

49. The method of any one of claims 30-48, wherein said applying electrical stimulation comprises applying non-invasive brain stimulation (NIBS) to a right VC region, a left VC region, or a combination thereof.

50. The method of any one of claims 30-49, further comprising assessing effectiveness of the electrical stimulation in said treating hypersomnia in the subject.

51. The method of claim 50, wherein said assessing comprises measuring a level of sleepiness or wakefulness of the subject using a visual-analog scale (VAS), a psychomotor vigilance test (PVT), a Likert scale, a Stanford Sleepiness Scale (SSS), a maintenance of wakefulness test (MWT), an Epworth sleepiness scale (ESS), or a multiple sleep latency test (MSLT).

52. The method of any one of claims 30-51, further comprising monitoring sleepiness or wakefulness of the subject using actigraphy or polysomnography.

53. A closed-loop method for treating insomnia in a subject, the method comprising:
recording a brain electrical signal associated with wakefulness of the subject using a first electrode, wherein the brain electrical signal is from a ventral capsule (VC) region, a subgenual cingulate (SGC) region, an amygdala region, an orbitofrontal cortex (OFC) region, or a striato-limbic region of the subject; and
applying electrical stimulation to a location in the VC region, the OFC region, or the SGC region of the subject using a second electrode in a manner effective to treat the insomnia in the subject when the electrical signal that is detected using the first electrode exceeds a threshold level.

54. The method of claim 53, wherein the location in the VC region is in a right VC region, a left VC region, or a combination thereof.

55. The method of claim 53 or 54, wherein said applying the electrical stimulation comprises applying electrical stimulation at a frequency in a range of 0.5 Hz to 10 Hz such that sleepiness of the subject is increased compared to sleepiness of the subject before said applying the electrical stimulation.

56. The method of any one of claims 53-55, wherein said applying the electrical stimulation comprises applying electrical stimulation for 5 seconds to 120 seconds.

57. The method of any one of claims 53-56, further comprising mapping the ventral capsule (VC) region, a subgenual cingulate (SGC) region, an amygdala region, an orbitofrontal cortex (OFC) region, or a striato-limbic region of the subject to identify an optimal location for positioning the first electrode for recording the brain electrical signal associated with wakefulness of the subject.

58. The method of any one of claims 53-57, further comprising mapping the VC region, the OFC region, or the SGC region of the subject to identify an optimal location for positioning the second electrode for delivery of the electrical stimulation to treat the insomnia in the subject.

59. The method of any one of claims 53-58, wherein the electrode is placed on a surface of the VC region, the OFC region, or the SGC region.

60. The method of any one of claims 53-59, wherein the electrode is placed within the VC region, the OFC region, or the SGC region.

61. The system of any one of claims 53-60, wherein the electrode is a non-brain penetrating surface electrode array or a brain-penetrating electrode array.

62. The method of any one of claims 53-61, further comprising using a control algorithm to automate said applying electrical stimulation when the brain electrical signal associated with wakefulness exceeds the threshold level.

63. The method of claim 62, wherein the control algorithm comprises a machine learning algorithm for classification of degree of wakefulness based on the brain electrical signal.

64. The method of claim 63, wherein the machine learning algorithm is a supervised machine learning algorithm.

65. The method of any one of claims 63-64, wherein the control algorithm further modulates one or more programmed stimulation parameters based on a level of power of the brain electrical signal.

66. The method of any one of claims 53-65, wherein the brain electrical signal associated with wakefulness comprises alpha frequency, beta frequency, gamma frequency, delta frequency, or theta frequency neural oscillations.

67. The method of claim 66, wherein the gamma frequency neural oscillations are low gamma frequency neural oscillations in a range from 31 Hz to 81 Hz or high gamma frequency neural oscillations in a range from 81 Hz to 150 Hz.

68. The method of claim 66 or 67, further comprising measuring a relative spectral power level for the gamma frequency neural oscillations or beta frequency neural oscillations, wherein higher relative power of the gamma frequency neural oscillations or beta frequency neural oscillations is associated with lower sleepiness and higher wakefulness compared to lower relative power of the gamma frequency neural oscillations or beta frequency neural oscillations.

69. The method of any one of claims 53-68, wherein the subject is monitored continuously or intermittently.

70. The method of any one of claims 53-69, wherein the electrical stimulation is applied unilaterally or bilaterally.

71. The method of any one of claims 53-70, wherein said applying electrical stimulation comprises applying non-invasive brain stimulation (NIBS) to the VC region, the OFC region, or the SGC region.

72. The method of any one of claims 53-71, wherein said applying electrical stimulation comprises applying non-invasive brain stimulation (NIBS) to a right VC region, a left VC region, or a combination thereof.

73. The method of any one of claims 53-72, further comprising assessing effectiveness of the electrical stimulation in treating the insomnia in the subject.

74. The method of claim 73, wherein said assessing comprises measuring a level of sleepiness or wakefulness of the subject using a visual-analog scale (VAS), a psychomotor vigilance test (PVT), a Likert scale, a Stanford Sleepiness Scale (SSS), a maintenance of wakefulness test (MWT), an Epworth sleepiness scale (ESS), a multiple sleep latency test (MSLT), or an Athens insomnia scale.

75. The method of any one of claims 53-74, further comprising monitoring sleepiness or wakefulness of the subject using actigraphy or polysomnography.

76. The method of any one of claims 53-75, wherein the insomnia is caused by deep brain stimulation therapy to an anterior limb of internal capsule (ALIC) region of the subject.

77. The method of claim 76, wherein the subject has obsessive-compulsive disorder or major depressive disorder.

78. A system for modulating sleep-wake levels in a subject, the system comprising:
a first electrode adapted for recording a brain electrical signal associated with wakefulness or sleepiness of the subject, wherein the brain electrical signal is from a ventral capsule (VC) region, a subgenual cingulate (SGC) region, an amygdala region, an orbitofrontal cortex (OFC) region, or a striato-limbic region of the subject;
a second electrode adapted for applying electrical stimulation to a location in the VC region, the OFC region, or the SGC region of the subject; and
a processor programmed to instruct the second electrode to apply the electrical stimulation to the VC region, the OFC region, or the SGC region of the subject in a manner effective to modulate sleep-wake levels in the subject when the brain electrical signal associated with wakefulness or sleepiness of the subject that is detected using the first electrode exceeds a threshold level.

79. The system of claim 78, wherein the system further comprises a user interface comprising an input electronically coupled to the processor for instructing the second electrode to apply the electrical stimulation to the location in the VC region, the OFC region, or the SGC region of the subject.

80. The system of claim 79, wherein the user interface is password protected and is operable by a health care practitioner.

81. The system of any one of claims 78-80, wherein the processor is further programmed to modulate one or more programmed stimulation parameters according to the algorithm's control law; and apply the modulated electrical stimulation to the VC region, the OFC region, or the SGC region of the subject using the second electrode in a manner effective to treat a sleep-wake disorder.

82. The system of any one of claims 78-81, wherein the processor is further programmed to apply the electrical stimulation at a frequency in a range from 70 Hz to 250 Hz such that wakefulness or energy of the subject is increased compared to wakefulness or energy of the subject before said applying the electrical stimulation.

83. The system of any one of claims 78-82, wherein the processor is further programmed to apply the electrical stimulation at a frequency in a range of 0.5 Hz to 10 Hz such that sleepiness of the subject is increased compared to sleepiness of the subject before said applying the electrical stimulation.

84. The system of any one of claims 78-83, wherein the processor is further programmed to apply the electrical stimulation for 5 seconds to 120 seconds.

85. The system of any one of claims 78-84, wherein the processor is further programmed to apply the electrical stimulation intermittently with a rest period between stimulations.

86. The system of claim 85, wherein the rest period ranges from 10 seconds to 10 minutes.

87. The system of claim 86, wherein the processor is further programmed to apply the electrical stimulation intermittently with a stimulation period of 6 seconds and a rest period of 10 seconds, or a stimulation period of 30 seconds and a rest period of 2 minutes.

88. The system of any one of claims 78-87, wherein the processor is further programmed to set a maximum number of electrical stimulations per day.

89. The system of any one of claims 78-88, wherein the processor is further programmed to set a total amount of time of electrical stimulation per day.

90. The system of any one of claims 78-89, wherein the first electrode is adapted for positioning on a surface of the VC region, the SGC region, the amygdala region, or the OFC region of the subject or within the VC region, the SGC region, the amygdala region, the OFC region, or the striato-limbic region of the subject.

91. The system of any one of claims 78-90, wherein the second electrode is adapted for positioning on a surface of the VC region, the OFC region, or the SGC region or within the VC region, the OFC region, or the SGC region.

92. The system of any one of claims 78-91, wherein the first electrode or the second electrode is a non-brain penetrating surface electrode array or a brain-penetrating electrode array.

93. The system of any one of claims 78-92, wherein the first electrode or the second electrode is a non-brain penetrating electrode.

94. The system of any one of claims 78-92, wherein the first electrode or the second electrode is a brain penetrating electrode.

95. The system of any one of claims 78-94, wherein the second electrode applies non-invasive brain stimulation (NIBS) to the VC region, the OFC region, or the SGC region.

96. The system of any one of claims 78-95, wherein the second electrode applies non-invasive brain stimulation (NIBS) to the right VC region, the left VC region, or a combination thereof.

97. The system of any one of claims 78-96, wherein the sleep-wake disorder is restless leg syndrome, non-rapid eye movement (NREM) sleep arousal disorder, rapid eye movement (REM) sleep behavior disorder.

98. The system of any one of claims 78-97, wherein the insomnia is caused by deep brain stimulation therapy to an anterior limb of internal capsule (ALIC) region of the subject.

99. The system of claim 98, wherein the subject has obsessive-compulsive disorder or major depressive disorder.

100. The system of any one of claims 78-99, wherein the processor is further programmed to set a maximum number of electrical stimulations per day.

101. The system of any one of claims 78-100, wherein the processor is further programmed to set a total amount of time of electrical stimulation per day.

102. A closed-loop method for treating obsessive-compulsive disorder or major depressive disorder in a subject, the method comprising:
applying electrical stimulation to an anterior limb of internal capsule (ALIC) region of the subject using a first electrode in a manner effective to treat the obsessive-compulsive disorder or major depressive disorder in the subject;
recording a beta frequency brain electrical signal associated with wakefulness of the subject using a second electrode, wherein the beta frequency brain electrical signal is from a striato-limbic region of the subject; and
decreasing amplitude of electrical stimulation during nighttime hours when spectral power of the beta frequency brain electrical signal exceeds a threshold level.

103. The method of claim 102, wherein the beta frequency brain electrical signal is in a range from 25 Hz to 30 Hz.

104. The method of claim 102 or 103, further comprising applying electrical stimulation in a range of 0.5 Hz to 10 Hz during nighttime hours when the spectral power of the beta frequency brain electrical signal exceeds the threshold level.
